(19) United States
(12) Patent Application Publication
(10) Pub. No.: US 2024/0150805 A1
(43) Pub. Date: May 9, 2024

(54) Glycosylated Opioids

(71) Applicant: River Stone Biotech ApS, Copenhagen (DE)

(72) Inventors: Jens Houghton-Larsen, Copenhagen (DE); Rubini Kannangara, Copenhagen (DE); Esben Halkjaer Hansen, Copenhagen (DE); Evan Chaberski, Copenhagen (DE); Laura Tatjer-Recordá, Copenhagen (DE)

(21) Appl. No.: 18/288,012

(22) PCT Filed: May 5, 2022
(86) PCT No.: PCT/EP2022/062130

§ 371 Date: Oct. 23, 2023

(30) Foreign Application Priority Data
May 7, 2021 (EP) 21172832.4

(51) Int. Cl. C12P 19/60; C12N 9/10; C12N 15/52

(52) U.S. Cl. CPC C12P 19/60; C12N 9/1051; C12N 15/52; C12Y 204/01262

(57) ABSTRACT

The present invention relate to a method for producing an oripavine glycoside and/or nororipavine glycoside comprising providing (i) a oripavine acceptor and/or nororipavine acceptor, (ii) a glycosyl donor, and (iii) a glycosyl transferase (UGT), and contacting the oripavine acceptor and/or nororipavine acceptor, the glycosyl donor, and the UGT at conditions allowing the UGT to transfer a glycosyl moiety from the glycosyl donor to the oripavine acceptor and/or nororipavine acceptor and thereby produce the oripavine glycoside and/or nororipavine glycoside.

### FIELD OF THE INVENTION

The present invention relates to methods for producing oripavine glycosides and/or nororipavine glycosides and to genetically modified host cells producing such glycosides. Also included are cultures of the genetically modified host cell and methods for cultivating such cultures into fermentation compositions and isolating produced oripavine glycosides and/or nororipavine glycosides therefrom in the formation of compositions comprising oripavine glycosides and/or nororipavine glycosides. The invention also relates to novel oripavine glycosides and/or nororipavine glycosides.

### BACKGROUND OF THE INVENTION

Enzymatic preparation of opioids such as benzylisoquinoline alkaloids (BIA's) using enzymes either in vitro or recombinantly in genetically modified host cells expressing such enzymes is known in the art. Examples are US2019100781, WO2019/165551 and WO2021/069714.

However, the turnover of enzymatic conversion of substrates/precursors into a desired opioid (both the rates and amounts) is determined by enzymatic kinetics of the multitude of enzymes involved in the production of a desired opioid, the equilibria of all the steps including transport along with negative product feed back on growth or reaction rates which can significantly impede the yields of opioids using such methods. Other drawbacks in current technology includes product toxicity or other product properties impeding growth of production cultures.

In addition, side effects of currently available opioids such as morphine and the like, such as addictions, toxicity and/or psychedelic effects call for the development of new opioids having improved properties for use in therapeutic regimes.

### SUMMARY OF THE INVENTION

The present invention provides improvements offering solutions to certain drawbacks of producing opioids using the known technology, particularly pronounced when producing the opioid precursors thebaine, northebaine, oripavine and/or nororipavine, precursors that are essential to many higher opioids. The invention also provides glycosyl transferases, which surprisingly acts to glycosylate opioids both in vitro and in vivo and thereby circumvent drawbacks of the known technology and moreover which also integrate and work in genetically modified host cells to produce therein opioid glycosides. The inventors have also found that glycosylation of these opioids not only produce hitherto unknown opioid glycosides, which possesses interesting properties, but in vivo expression of glycosyltransferases also offers a range of hitherto unknown advantages in processes of producing these in genetically modified cell factories, such as yeast, including but not limited, to avoiding futile ATP consuming cycle of repeated uptake (By proton symport) and excretion (possibly by ATP consuming efflux pumps) during the oripavine demethylation step; secretion and separation of nororipavine glycosides the cells which prevents (i) intracellular nororipavine degradation; (ii) acidification of the yeast cytosol and the stress associated with repeated cycles of excretion and proton driven uptake of Nororipavine; (iii) inhibition of oripavine uptake by unwanted competitive uptake of extracellular nororipavine; (iv) product inhibition of the oripavine demethylase enzyme by presence of high concentrations of nororipavine; and (v) increase in propagation and biomass production of genetically modified cell factories glycosylating oripavine and/or nororipavine. Accordingly, in a first aspect this invention provides a method for producing an oripavine and/or nororipavine glycoside comprising providing (i) a oripavine and/or nororipavine acceptor, (ii) a glycosyl donor, and (iii) a glycosyl transferase (UGT), and contacting the oripavine and/or nororipavine acceptor, the glycosyl donor, and the UGT at conditions allowing the UGT to transfer a glycosyl moiety from the glycosyl donor to the oripavine and/or nororipavine acceptor and thereby produce the oripavine and/or nororipavine glycoside.

In a further aspect the invention provides a glycoside comprising an oripavine and/or nororipavine aglycone and a glycosyl group.

In a further aspect the invention provides a microbial host cell genetically modified to produce an oripavine and/or nororipavine glycoside in the presense of a glycosyl donor, said cell expressing one or more heterologous genes encoding one or more UGTs, which in the presence of a glycosyl donor and a oripavine and/or nororipavine acceptor, transfers a glycosyl moiety from the glycosyl donor to the oripavine and/or nororipavine acceptor and thereby produce the oripavine and/or nororipavine glycoside.

In a further aspect the invention provides a cell culture, comprising host cell of the invention and a growth medium.

In a further aspect the invention provides a fermentation composition comprising the oripavine and/or nororipavine glycosides comprised in the cell culture of the invention.

In a final aspect the invention provides a composition comprising the fermentation liquid of the invention and/or the oripavine glycoside and/or nororipavine glycoside of the invention and one or more agents, additives and/or excipients.

### DESCRIPTION OF DRAWINGS AND FIGURES

The figures included herein are illustrative and simplified for clarity, and they merely show details which are essential to the understanding of the invention, while other details may have been left out. Throughout the specification, claims and drawings the same reference numerals are used for identical or corresponding parts. In the figures and drawing include herein:

FIG. 1 shows a stacked bar diagram showing values from two separate in vitro experiments. One experiment was done with UGTs and 500 μM oripavine as substrate, the other done with UGTs and 500 μM Nororipavine as substrate. Only the UGTs that show glucosylation activity on Oripavine and/or nororipavine are shown in the figure. Glucosylated Nororipavine abbreviated Nororipavine_Glu, and glucosylated oripavine abbreviated oripavine_Glu in legend.

FIG. 2 shows production of glucosylated Nororipavine and glucosylated oripavine in yeast strain sOD504 transformed by selected UGTs.

FIG. 3 shows dry weight biomass concentration during fed-batch fermentation of sOD507 and sOD515.

FIG. 4 shows the percentage increase in total Nororipavine production of a Nororipavine production strain expressing a UGT (strain sOD515) compared to an identical strain but without expression of a UGT (sOD507), during fed batch fermentation.

FIG. 5, FIG. 6 and FIG. 7 shows deglucosylation of nororipavine_glu in broth by glucosidase blends and 3 different sets of conditions.

FIG. 8 shows in vitro testing data of subfamily 72 UGTs in a stacked bar diagram combining results from two separate in vitro glucosylation experiments. One experiment with addition of 500 μM nororipavine and measurement of glucosylated nororipavine (nororipavine_Glu) and one experiment with addition of 500 μM oripavine and measurement of glucosylated oripavine (oripavine_Glu). Tested UGTs are listed on X-axis.

FIG. 9 shows the MS/MS spectra of nororipavine-glucosides in positive ion mode at the m/z indicated on the x-axis label.

FIG. 10 shows an enlarged region of a MS/MS spectrum for the nororipavine-glucoside fragment occurring at m/z 446.1820 in positive ion mode.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

The features and advantages of the present invention is readily apparent to a person skilled in the art by the below detailed description of embodiments and examples of the invention with reference to the figures and drawings included herein.

### Definitions

Any EC numbers than may be used herein refers to Enzyme Nomenclature 1992 from NC-IUBM B, Academic Press, San Diego, California, including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzyme.expasy.org/.

The term “E4P” as used herein refers to erythrose-4-phosphate.

The term “Aro4” as used herein refers to DAHP synthase catalyzing the reaction of PEP and E4P into DAHP.

The term “DAHP” as used herein refers to 3-deoxy-D-arabino-2-heptulosonic acid 7-phosphate.

The term “Aro1” as used herein refers to EPSP synthase catalyzing conversion of DAHP into EPSP.

The term “EPSP” as used herein refers to 5-enolpyruvylshikimate-3-phosphate.

The term “Aro2” as used herein refers to chorismate synthase catalyzing conversion of EPSP

into chorismate.

The term “Tyr1” as used herein refers to prephenate dehydrogenase catalyzing conversion of prephenate into 4-HPP

The term “4-HPP” as used herein refers to 4-hydroxyphenylpyruvate

The term “Aro8” and “Aro9” as used herein refers to aromatic aminotransferase reversibly catalyzing conversion of 4-HPP into L-tyrosine

The term “ARO10” or HPPDC as used herein refers to hydroxyphenylpyruvate decarboxylase catalyzing 4-HPP into 4-HPAA.

The term “4-HPAA” as used herein refers to 4-Hydroxyphenylacetaldehyde.

The term “TH” as used herein refers to a cytochrome P450 enzyme having tyrosine hydroxylase activity and converting L-tyrosine into L-DOPA.

The term “demethylase” as used herein refers to a P450 enzyme, capable of demethylating thebaine into northebaine, thebaine into oripavine, thebaine into nororipavine and/or oripavine into nororipavine.

The term “DRS” as used herein refers to 1,2-dehydroreticuline synthase, a cytochrome P450 enzyme which catalyze conversion of (S)-Reticuline into 1,2-dehydroreticuline.

The term “DRR” as used herein refers to 1,2-dehydroreticuline reductase which catalyzes conversion of 1,2-dehydroreticuline to (R)-Reticuline.

The term “DRS-DRR” as used herein refers to 1,2-dehydroreticuline synthase-1,2-dehydroreticuline reductase fused complex catalyzing conversion of (S)-Reticuline into (R)-reticuline. This complex may also be referred to as STORR or REPI. DRS-DRR or DRS together with DRR are also categorised as epimerases or isomerases.

The term “CPR” as used herein refers to a cytochrome P450 reductase catalyzing the electron transfer (from NADPH) to a cytochrome P450 enzyme of the pathway, typically in the endoplasmic reticulum of a eukaryotic cell. For distinction and as disclosed herein CPR's are divided into demethylase-CPR used for CPR's capable of reducing demethylases; DRS-CPR used for CPR's capable of reducing DRS and TH-CPR used for CPR's capable of reducing TH. Demethylase-CPR, DRS-CPR and TH-CPR may be identical or different, depending on the P450 to be reduced.

The term “Cytochrome P450 enzyme” or “P450 enzymes” or “P450” as used herein interchangeably refers to a family of monooxygenases enzymes containing heme as a cofactor. P450s are also known as “CYPs”. For distinction and as disclosed herein P450 enzymes are divided into demethylase P450s; DRS P450s, and TH P450s.

The term “family CYP6” as used herein about some demethylases refers to demethylases having >40% amino acid sequence identity to any known demethylase belonging to CYP6 family as defined by Nelson 2006, Cytochrome P450 Nomenclature, included herein by reference.

The term “family CYP76” as used herein about some THs refers to THs having tyrosine hydroxylase activity and capable of catalyzing L-tyrosine into L-DOPA.

The term “DODC” and TYDC″ as used herein refers to L-dopa decarboxylase and tyrosine decarboxylase respectively catalyzing conversion of L-DOPA into dopamine and tyrosine into 4-HPP.

The term “MAO” as used herein refers to monoamine oxidase catalyzing conversion of dopamine to 3,4 DHPAA

The term “DHPAA” as used herein refers to 3,4-dihydroxyphenylacetaldehyde.

The term “NCS” as used herein refers to Norcoclaurine synthase catalyzing conversion of dopamine and 4-HPAA into Norcoclaurine.

The term “6-OMT” as used herein refers to 6-O-methyltransferase catalyzing conversion of (S)-norcoclaurine to (S)-Coclaurine

The term “CNMT” as used herein refers to Coclaurine-N-methyltransferase catalyzing conversion of (S)-Coclaurine to (S)—N-Methylcoclaurine and/or (S)-3′-hydroxycoclaurine to (S)-3′-hydroxy-N-methyl-coclaurine.

The term “NMCH” as used herein refers to N-methylcoclaurine 3′-monooxygenase catalyzing conversion of (S)-Coclaurine to (S)-3′-hydroxycoclaurine and/or (S)—N-Methylcoclaurine to (S)-3′-Hydroxy-N-M ethylcoclaurine

The term “4′-OMT” as used herein refers to 3′-hydroxy-N-methyl-(S)-coclaurine 4′-O-methyltransferase catalyzing conversion of (S)-3′-Hydroxy-N-Methylcoclaurine to (S)-reticuline.

The term “SAS” as used herein refers to salutaridine synthase catalyzing conversion of (R)-reticuline to Salutaridine.

The term “SAR” as used herein refers to salutaridine reductase catalyzing conversion of Salutaridine to Salutaridinol.

The term “SAT” as used herein refers to salutaridinol 7-O-acetyltransferase catalyzing conversion of Salutaridinol to 7-O-acetylsalutaridinol.

The term “THS” as used herein refers to thebaine synthase catalyzing conversion of 7-O-acetylsalutaridinol into thebaine.

The term “BIA” or “benzylisoquinoline alkaloid” as used herein refers to a compound of the general formula A:

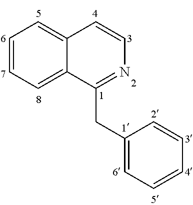

which is the structural backbone of many alkaloids with a wide variety of structures, or to alkaloid products deriving from formula A of the general formula B also known as morphinans:

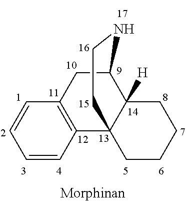

The terms “heterologous” or “recombinant” or “genetically modified” and their grammatical equivalents as used herein interchangeably refers to entities “derived from a different species or cell”. For example, a heterologous or recombinant polynucleotide gene is a gene in a host cell not naturally containing that gene, i.e. the gene is from a different species or cell type than the host cell. The terms as used herein about host cells refers to host cells comprising and expressing heterologous or recombinant polynucleotide genes.

The term “pathway” or “metabolic pathway” or “biosynthetic metabolic pathway” or “operative biosynthetic metabolic pathway” as used herein interchangeably is intended to mean one or more enzymes acting in a live cell to convert a chemical substrate into a chemical product. A pathway may include one enzyme or multiple enzymes acting in sequence. A pathway including only one enzyme may also herein be referred to as “bioconversion”, which is particularly relevant where a cell is fed with a precursor or substrate to be converted by the enzyme into a desired product molecule. Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds (co-factor and/or co-enzymes). The NADPH-dependent cytochrome P450 reductase (CPR) is an electron donor to cytochromes P450 (CYPs). CPR shuttles electrons from NADPH through the Flavin Adenine Dinucleotide (FAD) and Flavin Mononucleotide (FMN) coenzymes into the iron of the prosthetic heme-group of the CYP.

The term “in vivo”, as used herein refers to within a living cell or organism, including, for example animal, a plant or a microorganism.

The term “in vitro”, as used herein refers to outside a living cell or organism, including, without limitation, for example, in a microwell plate, a tube, a flask, a beaker, a tank, a reactor and the like.

The term “substrate” or “precursor”, as used herein refers to any compound that can be converted into a different compound. For example, thebaine can be a substrate for P450 and can be converted by demethuylation into Northebaine. For clarity, substrates and/or precursors include both compounds generated in situ by a enzymatic reaction in a cell or exogenously provided compounds, such as exogenously provided organic molecules which the host cell can metabolize into a desired compound.

Term “endogenous” or “native” as used herein refers to a gene or a polypeptide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

The terms “substantially” or “approximately” or “about”, if used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” if used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” is used herein about the relatedness between two amino acid sequences or between two nucleotide sequences.

The term “% identity” as used herein about amino acid or nucleotide sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{iden tical amino acid residues}{Length of alignment - total number of gaps in alignment} \times 100$$

The term “% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxyribonucleotides}{Length of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity. Alternatively, % identity for any candidate nucleic acid or amino acid sequence relative to a reference sequence can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31(13):3497-500.

Clustal Omega calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method:% age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The Clustal Omega output is a sequence alignment that reflects the relationship between sequences. Clustal Omega can be run, for example, at the Baylor College of Medicine Search Launcher site on the World Wide Web (searchlauncher.bcm.tmc.edu/multi-align/multi-align.html) and at the European Bioinformatics Institute site at http://www.ebi.ac.uk/Tools/msa/clustalo/. To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “mature polypeptide” or “mature enzyme” as used herein refers to a polypeptide in its final active form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide of the present invention. Host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

The term “glycosyl transferase” or “GT” as used herein refers to enzymes (EC2.4) that catalyze formation of glycosides by transfer of a glycosyl group (sugar) from an activated glycosyl donor to a nucleophilic glycosyl acceptor molecule, the nucleophile of which can be oxygen- carbon-, nitrogen-, or sulfur-based and in particular. The product of glycosyl transfer may be an O-, N-, S-, or C-glycoside. Glycosyltransferases may further be divided into different families depending on the 3D structure and reaction mechanism. More specifically the GT family 1 refers to UDP glycosyltransferases (UGTs) containing the PSPG box binding UDP-sugars. UGT-family members may further be divided into subfamilies and lower groupings as defined by the UGT Nomenclature Committee (Mackenzie et al., 1997) depending on the amino acid identity. Identities >40% belong to the same UGT-family e.g. UGT subfamily 71, 72 or 73, and amino acid identities >60% defines the subfamily groups e.g. UGT73Y.

The term “nucleotide glycoside” as used herein about glycosyl donors refers to compounds comprising a nucleotide moiety covalently linked to a glycosyl group, where the nucleotide comprise a nucleoside covalently linked to one or more phosphate groups. Such compounds are also referred to as “activated glycosides” and where the glycosyl group is a sugar as “nucleotide sugars” or “activated sugars”.

The terms “di-glycoside”, “tri-glycoside” and “tetra-glycoside” refer to molecules with 2, 3, and 4 glycoside moieties attached together at any 0-linkage.

### Method of Glycosylation

The invention provides a method for producing an oripavine and/or nororipavine glycoside comprising providing (i) a oripavine and/or nororipavine acceptor, (ii) a glycosyl donor, and (iii) a glycosyl transferase (UGT), and contacting the oripavine and/or nororipavine acceptor, the glycosyl donor, and the UGT at conditions allowing the UGT to transfer a glycosyl moiety from the glycosyl donor to the oripavine and/or nororipavine acceptor and thereby produce the oripavine and/or nororipavine glycoside.

The glycosyl donor is suitably a NTP-glycoside, aNDP-glycoside or a NMP-glycoside, wherein the the nucleoside is suitably selected from from Uridine, Adenosin, Guanosin, Cytidin and deoxythymidine. Such glycosyl donors include UDP-glycosides, ADP-glycosides, CDP-glycosides, CMP-glycosides, dTDP-glycosides and GDP-glycosides. In a particular embodiment the glycosyl donor is an NDP-glycoside, preferably having a Uridine nucleoside. Such glycosyl donors that are particularly useful are UDP-D-glucose (UDP-Glc), UDP-xylose (UDP-Xyl) or UDP-N-acetyl-D-glucosamine (UDP-GlcNAc).

Glycosyl transferases useful in the present invention include UGT's, in particular aglycone 0-UGTs. In some embodiments the UGT is an aglycone 0-glucosyltransferase.

Glycosyl transferases, such as UGTs, which comprises one or more amino acids G, H, I, I, H, G, I, L, S, H, G, N, at the positions corresponding to position 16, 17, 19, 169, 172, 173, 177, 200, 271, 360, 362, 364, 365, and 368 respectively of SEQ ID NO: 96 (non-gapped protein sequence) or conservative substitutions thereof are particularly useful for the method of the invention as it has been found that these have specificity for glycosylating oripavine and/or nororipavine. In some embodiments UGTs comprising amino acid G at the position corresponding to position 16 of SEQ ID NO: 96 are preferred, while additionally or alternatively in other embodiments UGTs comprising H at the position corresponding to position 17 of SEQ ID NO: 96 are preferred; while additionally or alternatively in still further embodiments UGT's comprising I or V at the position corresponding to position 19 of SEQ ID NO: 96 are preferred; while additionally or alternatively in still further embodiments UGTs comprising I or V at the position corresponding to position 169 of SEQ ID NO: 96 are preferred; while additionally or alternatively in still further embodiments UGTs comprising H at the position corresponding to position 172 of SEQ ID NO: 96 are preferred. In further additional or alternative embodiments UGTs comprising amino acids I, L, S or A at the position corresponding to position 177 of SEQ ID NO: 96 or amino acids G, S, H, G, N, S and E at the positions corresponding to position 173, 271, 360, 262, 364, 365 and 368 of SEQ ID NO: 96 are preferred. UGTs comprising any combination of these characteristic amino acids are also useful in the method of the invention. Also UGT's which have L, I or V at the position corresponding to position 200 of SEQ ID NO: 96 are attractive, more preferably L at position 200.

Further it has also been found that UGTs most useful for glycosylating oripavine or nororipavine does not comprise a R and/or M at the positions corresponding to position 17 and/or 177 respectively of SEQ ID NO: 96. Additionally or alternatively the UGT does not have any one of amino acids Q, Y, P, K, L or N at the position corresponding to position 172 of SEQ ID NO: 96. Additionally or alternatively the UGT does not have any one of amino acids M, S, or Q at the position corresponding to position 200 of SEQ ID NO: 96.

Accordingly, in one embodiment the UGT's described herein comprise one or more amino acids G, H, I, I, H, G, I, L, S, H, G, and/or N at the positions corresponding to position 16, 17, 19, 169, 172, 173, 177, 200, 271, 360, 362, 364, 365, and 368 respectively of SEQ ID NO: 96 or conservative substitutions thereof. Additionally or alternatively, in another embodiment the UGT's described herein comprise an amino acid G at the position corresponding to position 16 of SEQ ID NO: 96. Additionally or alternatively, in another embodiment the UGT's described herein comprise an amino acid H at the position corresponding to position 17 of SEQ ID NO: 96. Additionally or alternatively, in another embodiment the UGT's described herein comprise an amino acid I or V at the position corresponding to position 19 of SEQ ID NO: 96. Additionally or alternatively, in another embodiment the UGT's described herein comprise amino acids I or V at the position corresponding to position 169 of SEQ ID NO: 96. Additionally or alternatively, in another embodiment the UGT's described herein comprise amino acids I, L, S or A at the position corresponding to position 177 of SEQ ID NO: 96 or conservative substitutions thereof. Additionally or alternatively, in another embodiment the UGT's described herein comprise amino acid H at the position corresponding to position 172 of SEQ ID NO: 96. Additionally or alternatively, in another embodiment the UGT's described herein comprise amino acids G, L, S, H, G, N, S and E at the positions corresponding to position 173, 200, 271, 360, 262, 364, 365 and 368 of SEQ ID NO: 96. Additionally or alternatively, in another embodiment the UGT's described herein comprise amino acids L, I or V at the position corresponding to position 200 of SEQ ID NO: 96.

The UGT can in some embodiments be derived from a plant, in particular a plant selected from the genera of Quercus, optionally Quercus suber.

In further embodiments UGT is a subfamily 71 UGT (71-UGT), a subfamily 72 UGT (72-UGT) and/or a subfamily 73 UGT (73-UGT), which has been found to be effective at glycosylating oripavine and/or nororipavine, both in vitro and in vivo. Such UGTs suitably comprises an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 170, 172, 174, 176, 178, 180, 182, 184, 186, 188, 190, 192, 194, 196, 198, 200, 202, 204, 206, 208, 210, 212, 214, 216, 218, 220, or 222. The UGT of the invention is suitably a good performer regarding activity and/or specificity towards Oripavine and/or nororipavine. Such UGTs suitably comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 63, 77, 81, 82, 83, 84, 86, 87, 92, 93, 94, 95, 96, 97, 98, 99, 100, 101, 102, 103, 104, 105, 107, 108, 111, 112, 115, 116, 117, 170, 172, 174, 176, 178, 180, 182, 184, 186, 188, 190, 192, 194, 196, 198, 200, 202, 204, 206, 208, 210, 212, 214, 216, 218, 220, or 222.

In some embodiments the UGT of the invention has superior activity such as UGTs comprising an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 63, 83, 84, 86, 87, 101, 102, 103, 104, 105, 115, 170, 172, 174, 176, 178, 180, 182, 184, 186, 188, 190, 192, 194, 196, 198, 200, 202, 204, 206, 208, 210, 212, 214, 216, 218, 220, or 222.

In other embodiments the UGT of the invention has a superior specificity towards nororipavine which is at least 50% higher, such as at least 75% higher, such as at least 90% higher than the specificity towards oripavine, when performing the glycosylation in aqeuous tris buffer at pH 7,4 at 30° C. and at 0.5 mM substrate level. Such nororipavine specific UGTs include those comprising an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 77, 81, 82, 92, 93, 94, 95, 96, 97, 98, 99, 100, 102, 103, 104, 105, 107, 108, 116, or 117. Among nororipavine specific UGTs those also having superior activity are particularly useful such as the UGTs comprising an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 102, 103, 104, or 105.

In other embodiments the UGT of the invention has a superior specificity towards oripavine which is at least 50% higher, such as at least 75% higher, such as at least 90% higher than the specificity towards nororipavine, when performing the glycosylation in aqueous tris buffer at pH 7,4 at 30° C. and at 0.5 mM substrate level. Such oripavine specific UGTs include those which has at least 60%, such as at least 70%, such at least 75%, such as at least 80%, such as at least 90%, such as at least 95%, such as at least 99%, such as 100% identity to a UGT comprised in anyone of SEQ ID NO: 111, 112, or 115. Among oripavine specific UGTs those also having superior activity are particularly useful such as the UGTs comprising an amino acid sequence which has at least 60%, such as at least 70%, such at least 75%, such as at least 80%, such as at least 90%, such as at least 95%, such as at least 99%, such as 100% identity to a UGT comprised in SEQ ID NO: 115.

In separate embodiments the UGT of the invention is a subfamily 71 UGT which comprises an amino acid sequence which has at least 60%, such as at least 70%, such at least 75%, such as at least 80%, such as at least 90%, such as at least 95%, such as at least 99%, such as 100% identity to a 71-UGT comprised in anyone of SEQ ID NO: 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90. Among the subfamily 71 UGTs those also having superior performance are preferred, such as those comprising an amino acid sequence which has at least 60%, such as at least 70%, such at least 75%, such as at least 80%, such as at least 90%, such as at least 95%, such as at least 99%, such as 100% identity to a 71-UGT comprised in anyone of SEQ ID NO: 63, 77, 81, 82, 83, 84, 86, or 87.

In further separate embodiments the UGT of the invention is a subfamily 72 UGT which comprises an amino acid sequence which has at least 60%, such as at least 70%, such at least 75%, such as at least 80%, such as at least 90%, such as at least 95%, such as at least 99%, such as 100% identity to a 72-UGT comprised in anyone of SEQ ID NO: 91, 92, 93, 94, 95, 96, 97, 98, 99, 100, 101, 102, 103, 104, 105, 106, 107, or 108. Among the subfamily 72 UGTs those also having superior performance are preferred, such as those comprising an amino acid sequence which has at least 60%, such as at least 70%, such at least 75%, such as at least 80%, such as at least 90%, such as at least 95%, such as at least 99%, such as 100% identity to a 72-UGT comprised in anyone of SEQ ID NO: 92, 93, 94, 95, 96, 97, 98, 99, 100, 101, 102, 103, 104, 105, 107, or 108.

In still further separate embodiments the UGT of the invention is a subfamily 73 UGT which comprises an amino acid sequence which has at least 60%, such as at least 70%, such at least 75%, such as at least 80%, such as at least 90%, such as at least 95%, such as at least 99%, such as 100% identity to a 73-UGT comprised in anyone of SEQ ID NO: 109, 110, 111, 112, 113, 114, 115, 116, 117, 118, 119, 120. Among the subfamily 73 UGTs those also having superior performance are preferred, such as those comprising an amino acid sequence which has at least 60%, such as at least 70%, such at least 75%, such as at least 80%, such as at least 90%, such as at least 95%, such as at least 99%, such as 100% identity to a 73-UGT comprised in anyone of SEQ ID NO: 111, 112, 115, 116, or 117.

The method of the invention may also include one or more demethylation steps selected from
a) converting thebaine to oripavine;
b) converting thebaine to northebaine;
c) converting oripavine to nororipavine; and/or
d) converting northebaine to nororipavine;
   by contacting the thebaine, northebaine and/or oripavine with one or more 0-demethylases and/or N-demethylases. Suitable demethylases for use in such demethylation include those comprising and amino acid sequence which has at least 60%, such as at least 70%, such at least 75%, such as at least 80%, such as at least 90%, such as at least 95%, such as at least 99%, such as 100% identity to a to a demethylase comprised in any one of SEQ ID NO: 153, 155, 157, 256, or 258. Other examples which works remarkably well in converting thebaine and/or oripavine with low formation of by-products in a heterologous host cell includes the insect demethylases disclosed in WO2021069714 as SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869, incorporated herein by reference.

The demethylase activity may further be enhanced or co-factored by a demethylase-CPR capable of reducing the demethylase after demethylation, such as a demethylase-CPR comprising and amino acid sequence which has at least 60%, such as at least 70%, such at least 75%, such as at least 80%, such as at least 90%, such as at least 95%, such as at least 99%, such as 100% identity to a to the demethylase-CPR comprised in anyone of SEQ ID NO: 159, 161, or 260. One skilled in the art would recognize that other P450 demethylases and CPR pairs would work for production of nororipavine from oripavine, for example SEQ ID NO: 292, 294, 296, 298, 300 or 302 described in WO2021069714. In a particular embodiment the conversion rate (units mass⁻¹ time⁻¹) of the demethylase is increased compared to the conversion rate absent any UGT's converting the oripavine and/or nororipavine and the glycosyl donor into the corresponding oripavine and/or nororipavine glycoside.

### Method of Deglycosylation

In another aspect invention provides a method for producing a oripavine- and/or nororipavine aglycone comprising providing (i) a oripavine and/or nororipavine glycoside and (ii) a glycosidases and contacting the oripavine and/or nororipavine glycoside and (ii) with the glycosidase at conditions allowing the glycosidase to catalyze separation of a glycosyl moiety from the oripavine and/or nororipavine glycoside and thereby produce the oripavine and/or nororipavine aglycone. The glycosidase may suitably be a β-glycosidase, such as a β-glucosidase.

The deglycosylation method can also include any feature or combination of features of the glycolation method of the invention.

In the glycosylation method or the deglycosylation method of the invention the contacting of the oripavine and/or nororipavine acceptor, the glycosyl donor, and the UGT or the oripavine-glycoside and/or nororipavine-glycoside and the glycosidase is may suitably be made in a buffered aqueous solution at a pH from 4,0 to 8,5 and at a temperature of 10 to 85° C.

### Glycosylated Oripavines and Nororipavines

In a further aspect the invention also provides oripavine and/or nororipavine glycosides. Such glycosides are suitably oripavine-O-glycosides or nororipavine-O-glycosides. The glycosyl group is preferably glucose and the corresponding glycoside an oripavine-O-glucoside or a nororipavine-O-glucoside.

### Nororipavine-O-Glucoside:

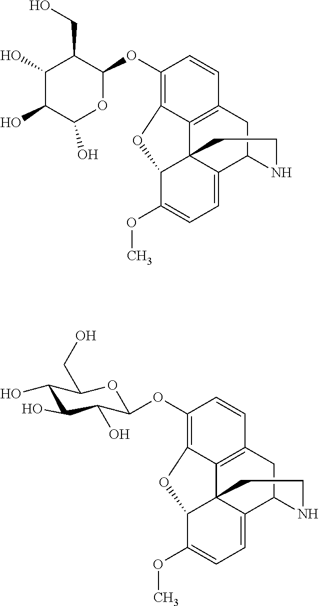

### Oripavine-O-Glucoside:

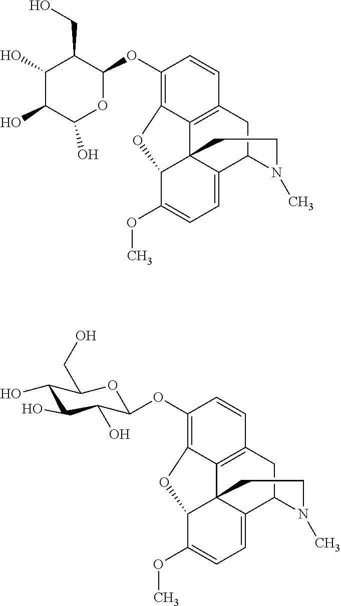

Genetically Modified Host Cells Producing Glycosylated Oripavine and/or Glycosylated Nororipavine

In a further aspect of the invention the method as described, supra, is performed in a host cell genetically modified to produce oripavine an/or nororipavine glycosides. Accordingly, the present invention also provides host cells, which are genetically modified to produce an oripavine glycoside and/or nororipavine glycoside in the presense of a glycosyl donor, wherein the host cell expresses one or more heterologous genes encoding one or more UGT's, which in the presence of a glycosyl donor and a oripavine acceptor and/or nororipavine acceptor, transfers a glycosyl moiety from the glycosyl donor to the oripavine acceptor and/or nororipavine acceptor and thereby produce the oripavine glycoside and/or nororipavine glycoside. UGT's and glycosyl donors for performing the method in the genetically modified cell are suitably those described, supra, for the method.

In some embodiment the host cell further comprises genes of an operative biosynthetic pathway which produces the oripavine acceptor and/or nororipavine acceptor and even the glycosyl donor. Operative biosynthetic pathways capable of producing the oripavine acceptor and/or nororipavine acceptor, suitably comprises one or more polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a chorismate synthase converting EPSP into Chorismate;
e) a chorismate mutase converting Chorismate into prephenate;
f) a prephenate dehydrogenase (Tyr1) converting prephenate into 4-HPP;
g) an aromatic aminotransferase converting 4-HPP into L-Tyrosine;
h) a tyrosine hydroxylase (TH) converting L-tyrosine into L-dopa
i) a TH-CPR capable of reducing the TH of h);
j) a L-dopa decarboxylase (DODC) converting L-dopa into dopamine;
k) a Tyrosine decarboxylase (TYDC) converting L-dopa into dopamine;
l) a hydroxyphenylpyruvate decarboxylase (HPPDC) converting 4-HPP into 4-HPPA;
m) a monoamine oxidase converting dopamine into 3,4-DHPAA;
n) a norcoclaurine synthase (NCS) converting Dopamine and 4-HPAA into (S)-norcoclaurine;
o) a 6-O-methyltransferase (6-OMT) converting (S)-norcoclaurine into (S)-Coclaurine and/or norlaudanosoline into (S)-3′-Hydroxy-coclaurine;
p) a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
q) a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
r) a 3′-hydroxy-N-methyl-(S)-coclaurine 4′-O-methyltransferase (4′-OMT) converting (S)-3′-Hydroxy-N-Methylcoclaurine into (S)-Reticuline;
s) a 1,2-dehydroreticuline synthase-1,2-dehydroreticuline reductase (DRS-DRR) converting (S)-Reticuline into (R)-reticuline;
t) a salutaridine synthase (SAS) converting (R)-reticuline into Salutaridine;
u) a salutaridine reductase (SAR) converting Salutaridine to Salutaridinol;
v) a salutaridinol 7-O-acetyltransferase (SAT) converting Salutaridinol into 7-O-acetylsalutaridinol;
w) a thebaine synthase (THS) converting 7-O-acetylsalutaridinol or 7-O-acetylsalutaridinol acetate into thebaine;
x) a demethylase converting thebaine into oripavine, thebaine into northebaine, oripavine into nororipavine and/or northebaine into nororipavine; and/or
y) a demethylase-CPR capable of reducing the demethylase of x).

In some embodiments, the corresponding:
a) DAHP synthase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DAHP synthase comprised in SEQ ID NO: 121;
b) chorismate mutase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the chorismate synthase comprised in SEQ ID NO: 123;
c) prephenate dehydrogenase (Tyr1) has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the DAHP synthase comprised in SEQ ID NO: 125;
d) Tyrosine Hydroxylase (TH) has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the TH comprised in SEQ ID NO: 127;
e) TH-CPR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the TH-CPR comprised in SEQ ID NO: 129;
f) DODC has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the DODC comprised in SEQ ID NO: 131;
g) Norcoclaurine synthase (NCS) has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the NCS comprised in SEQ ID NO: 133;
h) 6-OMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the 6-OMT comprised in SEQ ID NO: 135;
i) CNMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the CNMT comprised in SEQ ID NO: 137;
j) NMCH has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the NMCH comprised in SEQ ID NO: 139;
k) 4′-OMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the 4′-OMT comprised in SEQ ID NO: 141;
l) DRS-DRR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the VRS_DDR comprised in SEQ ID NO:143;
m) SAS has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the SAS comprised in SEQ ID NO: 145;
n) SAT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the SAR comprised in SEQ ID NO: 147;
o) SAR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the SAT comprised in SEQ ID NO: 149;
p) THS has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the THS comprised in SEQ ID NO: 151;

q) Demethylase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the demethylase comprised in anyone of SEQ ID NO: 153, 155, 157, 256, or 258 and
r) Demethylase-CPR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the demethylase-CPR comprised in anyone of SEQ ID NO: 159, 161, or 260.

Further suitable enzymes of the benzylisoquinoline alkaloid pathway are disclosed in U52019100781, WO2019/165551 and WO2021/069714, which are hereby incorporated by reference in their entirety.

In a particular embodiment the host cell of the invention, further comprises one or more demethylases converting thebaine into oripavine, thebaine into northebaine, oripavine into nororipavine and/or northebaine into nororipavine; optionally a demethylase which has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to a demethylase comprised in SEQ ID NO: 153, 155, 157, 256, or 258. Many more demethylases useful in the method and host cell of the present invention is disclosed in WO2021069714, incorporated by reference.

In some embodiments the conversion rate (unit mass-1 time-1) of one or more pathway enzymes is increased compared to the conversion rate absent any UGT's converting the oripavine and/or nororipavine and the glycosyl donor into the corresponding oripavine glycoside and/or nororipavine glycoside.

In another embodiment the host cell of the invention expresses one or more heterologous genes encoding transporter protein. The transporter protein of the invention may suitably be any natural or mutant tranporter protein capable of uptake or export in the host cell of a metabolite of the benzylisoquinoline alkaloid pathway disclosed herein, in particular of thebaine, northebaine, oripavine and/or nororipavine and/or their glycosides. Such transporter proteins may be a permease, such as a Purine Uptake Permease (PUP). As one example the transporter protein has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to the transporter comprised in SEQ ID NO: 224, 226, 228, 230, 232, 234, 236, 238, 240, 242, 244, 246, 248, 250, 252, or 254. In some embodiments, some transporters may reduce production of glycosylated product, eg. by transporting intermediates out of the cell. Expression of such transporters including the transporter comprised in SEQ ID NO: 163 can suitably be knocked out such as described in WO2019243624 to Valorbec. Many more transporters useful in the method and host cell of the present invention is disclosed in WO2021069714 or WO2020/078837, incorporated by reference. In particular for oripavine to nororipavine the demethylation reaction table 14-1 of WO2021069714 details numerous permeases provides over 10-fold improvement of in such demethylation reactions including but not limited to PUPs selected from the group of T109 Gf PUP3 83; T115 Csa PIJ P3 4 8; T116 HanPUP3 56; T122 PsoPUP3 17; T125 JcuPUP3 41; T126 CpePUP3 49; T130 NdoPUP3 89; T132 CmiPUP3 10; T133 PsoPUP3 18; T136 RchPUP3 42; T138 AduPUP3 58; T141 EcaPUP3 88; T142 McoPUP3 4; T143 CmiPUP3; T144 PsoPUP3 19; T149 Ace PU P3 59; T150 PsoPUP3 67; T151 PBLPUP3 75; T152 GRPUP3 87; T154 CmiPUP3 12; T157 RchPUP 36; T161 PsoPUP3 68; T162 PmiPUP3 76; T165 AcoPUP3 13; T168 FvePUP3 37; T169 ZjuPUP3 45; T170 LsaPUP3 53; T172 AcoPUP3 69; T177 PsoPUP3 22; T178 PsoPUP3 30; T180 McoPUP3 46; T181 HanPUP3 54; T182 CpaPUP3 62; T192 CmiPUP3 47; T193 AanPUP3 55; and T195 JcuPUP3 71. In particular PUP's of SEQ ID NO: 234, 236, 224, 226, 238, 240, 242, 228, 230, and 232 are desirable for providing improvements in the demethylase-mediated bioconversion of oripavine to nororipavine for some in the range of 100 to 10.000%, such as 250 to 7.500%, such as 500 to 5000%, such as 750 to 2500%, such as 1000 to 2000%, such as 1400-1662%. Such PUP's have been found to be suitable for use with production of glycosylated nororipavine.

Functional homologs (also known as functional variants) of the enzymes/polypeptides described herein are also suitable for use when producing oripavine glycosides and/or nororipavine glycosides by the method of the invention. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide. Functional homologs can be identified by analysis of nucleotide and polypeptide sequence alignments. For example, performing a query on a database of nucleotide or polypeptide sequences can identify homologs of benzylisoquinoline alkaloid biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability for benzylisoquinoline alkaloid biosynthesis. Amino acid sequence similarity allows for conservative amino acid substitutions, such as substitution of one hydrophobic residue for another or substitution of one polar residue for another. If desired, manual inspection of such candidates can be carried out to narrow the number of candidates to be further evaluated. Manual inspection can be performed by selecting those candidates that appear to have domains present in polypeptides in the benzylisoquinoline alkaloid biosynthetic pathway, e.g., conserved functional domains. In some embodiments, nucleic acids and polypeptides are identified from transcriptome data based on expression levels rather than by using BLAST analysis. Methods for conservative substitution is known to the skilled person, see for example https://www.ncbi.nlm.nih.gov/pmc/articles/PMC1449787/ or https://link.springer.com/article/10.1007/BF02300754. Conserved regions can be identified by locating a region within the primary amino acid sequence of a benzylisoquinoline alkaloid biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on for example the World Wide Web at sanger.ac.uk/Software/Pfam/ and pfam.janelia.org/. The information included at the Pfam database is described in Sonnhammer et al. (1998); Sonnhammer et al. (1997); and Bateman et al. (1999). Conserved regions also can be determined by aligning sequences of the same or related polypeptides from closely related species. Closely related species preferably are from the same family. In some embodiments, alignment of sequences from two different species is adequate to identify such homologs. Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity. For example, polypeptides suitable for producing oripavine glycosides and/or nororipavine glycosides in the genetically modified host cell of the invention functional homologs of TH's, NCS's, 6-OMT's, CNMT's, NMCH's, 4′-OMT's, DRS-DRR's, SAS's, SAR's, SAT's, THS's, CPR's, demethylating P450's, and UGTs. Methods to modify the substrate specificity of benzylisoquinoline alkaloids pathway enzymes are known to those skilled in the art and include without limitation site-directed/rational mutagenesis approaches, random directed evolution approaches and combinations in which random mutagenesis/saturation techniques are performed near the active site of the enzyme. For example see Osmani et al. (2009). A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. It will be appreciated that functional benzylisoquinoline alkaloids pathway enzymes/polypeptides and UGTs can include additional amino acids that are not involved in the enzymatic activities carried out by the enzymes. In some embodiments, such enzymes are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. In some embodiments, a nucleic acid sequence encoding a benzylisoquinoline alkaloids pathway enzyme/polypeptide can include a tag sequence that encodes a “tag” designed to facilitate subsequent manipulation (e.g., to facilitate purification or detection), secretion, or localization of the encoded enzyme. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, CT). Other examples of tags include a chloroplast transit peptide, a mitochondrial transit peptide, an amyloplast peptide, signal peptide, or a secretion tag. In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, a benzylisoquinoline alkaloids pathway enzyme/polypeptide is altered by domain swapping. In some embodiments the fused protein can comprise a glycosyl transferase as described herein fused (optionally by N-terminal fusion) to one or more moieties (anchors) comprising an Endoplasmic Reticulum localization site and providing for keeping the glycosyl transferase in close proximity to an Endoplasmic Reticulum membrane of the host cell, optionally via one or more linker moieties. Such anchors can provide for improved specificity, activity or folding of the glycosyl trasferase or a combination thereof. The fused protein can alternatively or additionally comprise a glycosyl transferase as described herein fused to one or more moieties providing for increased solubility (solubility tags) of the glycosyl transferase, optionally via one or more linker moieties. Such anchors can be either one of the anchor moieties comprised in SEQ ID NO: SEQ ID NO: SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, or SEQ ID NO: 202. Some anchors are particularly useful and beneficial in glycosylation of benzylisoquinoline alkaloid nor compounds like nororipavine, where it can boost the conversion rate considerably. The linker moieties can be either one of the linkers comprised in SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, or SEQ ID NO: 202, while the solubility tag can be either one of the tags comprised in SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, or SEQ ID NO: 222. In further additional or alternative embodiments surprisingly high production of glycosylated benzylisoquinoline alkaloid nor compounds like nororipavine can be achieved in host cell described herein comprising both a glycosyl transferase, optionally comprised in anyone of SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: SEQ ID NO: SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, or SEQ ID NO: 222, and a transporter, optionally comprised in anyone of SEQ ID NO: 224, 226, 228, 230, 232, 234, 236, 238, 240, 242, 244, 246, 248, 250, 252, or 254.

In further embodiments the host cell of the invention expresses one or more genes selected from the group of:
a) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the DAHP synthase encoding polynucleotide comprised in SEQ ID NO: 122 or genomic DNA thereof;
b) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the chorismate mutase encoding polynucleotide comprised in SEQ ID NO: 124 or genomic DNA thereof;
c) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the prephenate dehydrogenase encoding polynucleotide comprised in SEQ ID NO: 126 or genomic DNA thereof;
d) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the TH encoding polynucleotide comprised in SEQ ID NO: 128 or genomic DNA thereof;
e) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the TH-CPR encoding polynucleotide comprised in SEQ ID NO: 130 or genomic DNA thereof;
f) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the DODC encoding polynucleotide comprised in SEQ ID NO: 132 genomic DNA thereof;
g) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the NCS encoding polynucleotide comprised in SEQ ID NO: 134 or genomic DNA thereof;
h) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the 6-OMT encoding polynucleotide comprised in SEQ ID NO: 136 or genomic DNA thereof;
i) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the CNMT encoding polynucleotide comprised in SEQ ID NO: 138 or genomic DNA thereof;
j) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the NMCH encoding polynucleotide comprised in SEQ ID NO: 140 or genomic DNA thereof;
k) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the 4′-OMT encoding polynucleotide comprised in SEQ ID NO: 142 or genomic DNA thereof;
l) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the DRS-DRR encoding polynucleotide comprised in SEQ ID NO: 144 or genomic DNA thereof;
m) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the SAS encoding polynucleotide comprised in SEQ ID NO: 146 or genomic DNA thereof;
n) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the SAT encoding polynucleotide comprised in SEQ ID NO: 148 or genomic DNA thereof;
o) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the SAR encoding polynucleotide comprised in SEQ ID NO: 150 or genomic DNA thereof;
p) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the THS encoding polynucleotide comprised in SEQ ID NO: 152 or genomic DNA thereof;
q) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the demethylase encoding polynucleotide comprised in anyone of SEQ ID NO: 154, 156, 158, 255, or 257 or genomic DNA thereof;
r) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the demethylase-CPR encoding polynucleotide comprised in any one of SEQ ID NO: 160, 162, or 259 or genomic DNA thereof; and
s) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identical to the transporter encoding polynucleotide comprised in SEQ ID NO: 223, 225, 227, 229, 231, 233, 235, 237, 239, 241, 243, 245, 247, 249, 251, or 253 or genomic DNA thereof.

Any gene disclosed herein may be codon optimized for expression in a particular selected host using methods available to the skilled person or commercially available from technology providers—see for example Gene Reports (2017), incorporated herein by reference.

The host cell of the invention may be any host cell suitable for hosting and expressing the UGT's glycosylating the oripavine acceptor and/or nororipavine acceptor and optionally other polypeptides of the operative biosynthetic pathways producing the oripavine acceptor and/or nororipavine acceptor. In particular the host cell of the invention may be a eukaryote cell selected from the group consisting of mammalian, insect, plant, or fungal cells. In another embodiment the cell is a fungal cell selected from the phylas consisting of Ascomycota, Basidiomycota, Neocallimastigomycota, Glomeromycota, Blastocladiomycota, Chytridiomycota, Zygomycota, Oomycota and Microsporidia. A particularly useful fungal cell is a yeast cell selected from the group consisting of ascosporogenous yeast (Endomycetales), basidiosporogenous yeast, and Fungi Imperfecti yeast (Blastomycetes). Such yeast cells may further be selected from the genera consisting of Saccharomyces, Kluveromyces, Candida, Pichia, Debaromyces, Hansenula, Yarrowia, Zygosaccharomyces, and Schizosaccharomyces. More specifically the yeast cell may be selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.

An alternative fungal host cell of the invention is a filamentous fungal cell. Such filamentous fungal cell may be selected from the phylas consisting of Ascomycota, Eumycota and Oomycota, more specifically selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma. In important embodiments the filamentous fungal cell may be selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

In one embodiment the cell is a plant cell for example of the genus Physcomitrella or Papaver, in particular Papaver somniferum. Other plant cells can be of the family Solanaceae, such genuses of Nicotiana, such as Nicotiana benthamiana. In addition to plant cells the invention also provides an isolated plant, e.g., a transgenic plant, plant part comprising the benzylisoquinoline alkaloid pathway polypeptides of the invention and producing the benzylisoquinoline alkaloids of the invention in useful quantities. The compound may be recovered from the plant or plant part. The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats. Also included within the scope of the present invention is any the progeny of such plants, plant parts, and plant cells. The transgenic plant or plant cells comprising the operative pathway of the invention and produce the compound of the invention may be constructed in accordance with methods known in the art. In short, the plant or plant cell is constructed by incorporating one or more expression vectors of the invention into the plant host genome or chloroplast genome and propagating the resulting modified plant or plant cell into a transgenic plant or plant cell. The expression vector conveniently comprises the polynucleotide construct of the invention. The choice of regulatory sequences, such as promoter and terminator sequences and optionally signal or transit sequences, is determined, for example, on the basis of when, where, and how the pathway polypeptides is desired to be expressed. For instance, the expression of a gene encoding a pathway enzyme polypeptide may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506. For constitutive expression, the 358-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pint promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals. A promoter enhancer element may also be used to achieve higher expression in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression. The selectable marker gene and any other parts of the expression construct may be chosen from those available in the art. The polynucleotide construct or expression vector is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274). Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mo/. Biol. 21: 415-428. Additional transformation methods include those described in U.S. Pat. Nos. 6,395,966 and 7,151,204 (both incorporated herein by reference in their entirety). Following transformation, the transformants having incorporated the expression vector or polynucleotide construct of the invention are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase. In addition to direct transformation of a particular plant genotype with a polynucleotide construct of the invention, transgenic plants may be made by crossing a plant comprising the construct to a second plant lacking the construct. For example, a polynucleotide construct encoding a glycosyl transferease of the invention can be introduced into a particular plant variety by crossing, without the need for ever directly transforming a plant of that given variety. Therefore, the invention encompasses not only a plant directly regenerated from cells which have been transformed in accordance with the invention, but also the progeny of such plants. As used herein, progeny may refer to the offspring of any generation of a parent plant prepared in accordance with the present invention. Such progeny may include a polynucleotide construct of the invention. Crossing results in the introduction of a transgene into a plant line by cross pollinating a starting line with a donor plant line. Non-limiting examples of such steps are described in U.S. Pat. No. 7,151,204. Plants may be generated through a process of backcross conversion. For example, plants include plants referred to as a backcross converted genotype, line, inbred, or hybrid. Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The host cell of the invention may, depending on the choice of strain be even further modified by one or more of
a) attenuating, disrupting and/or deleting one or more native or endogenous genes of the cell;
b) Overexpressing polynucleotides encoding the UGTs and optionally one or more of the polypeptides comprised in the operative metabolic pathway of the invention by inserting two or more copies of the respective coding genes;
c) increasing the amount of a substrate for at least one polypeptide of the operative metabolic pathway; and/or
d) increasing tolerance towards one or more substrates, intermediates, or product molecules from the operative metabolic pathway.

Useful host cell may have one or more native or endogenous genes attenuated, disrupted and/or deleted. Where the host cell is a yeast strain is is useful to attenuate, disrupt and/or delete one or more dehydrogenases or reductases native to the host cell, particularly those comprised in anyone of SEQ ID NO: 165 or 167 or any of its paralogs or orthologs having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99% identity to anyone of SEQ ID NO: 165 or 167. The expressed UGT amy also suitably be absent a signal peptide targeting the UGT for secretion.

### Gene Constructs and Expression Vectors

In a separate aspect the invention also disclosed herein are polynucleotide constructs comprising a polynucleotide sequence encoding a UGT or polypeptide comprised in the operative metabolic pathway of the invention operably linked to one or more control sequences, which direct heterologous expression of the UGT or pathway polypeptide in the host cell harbouring the polynucleotide construct. Conditions for the expression should be compatible with the control sequences. In particular, the control sequence is heterologous to the UGT or pathway polypeptide and in one embodiment the polynucleotide sequence encoding the UGT or pathway polypeptide and the control sequence are both heterologous to the host cell comprising the construct. In one embodiment the polynucleotide construct is an expression vector, comprising the polynucleotide sequence encoding the heterologous enzyme or transporter protein of the invention operably linked to the one or more control sequences.

Polynucleotides may be manipulated in a variety of ways allow expression of the heterologous enzyme or transporter protein. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may also be an inducible promoter.

Examples of suitable promoters for directing transcription of the nucleic acid construct of the invention in fungal host cell are promoters obtained from the genes for Aspergillus nidulans acetamidase, Aspergillus niger neutral α-amylase, Aspergillus niger acid stable α-amylase, Aspergillus niger or Aspergillus awamori glucoamylase (glaA), Aspergillus gpdA promoter, Aspergillus oryzae TAKA amylase, Aspergillus oryzae alkaline protease, Aspergillus oryzae triose phosphate isomerase, A. niger or A. awamori endoxylanase (xlnA) or β-xylosidase (xlnD), Fusarium oxysporum trypsin-like protease (WO 96/00787), Fusarium venenatum amyloglucosidase (WO2000/56900), Fusarium venenatum Dania (WO 00/56900), Fusarium venenatum Quinn (WO 00/56900), Rhizomucor miehei lipase, Rhizomucor miehei aspartic proteinase, Trichoderma reesei β-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei β-xylosidase, as well as the NA2-tpi promoter and mutant, truncated, and hybrid promoters thereof. NA2-tpi promoter is a modified promoter from an Aspergillus neutral α-amylase gene in which the untranslated leader has been replaced by an untranslated leader from an Aspergillus triose phosphate isomerase gene. Examples of such promoters include modified promoters from an Aspergillus niger neutral α-amylase gene in which the untranslated leader has been replaced by an untranslated leader from an Aspergillus nidulans or Aspergillus oryzae triose phosphate isomerase gene. Other examples of promoters are the promoters described in WO2006/092396, WO2005/100573 and WO2008/098933, incorporated herein by reference.

Examples of suitable promoters for directing transcription of the nucleic acid construct of the invention in a yeast host include the glyceraldehyde-3-phosphate dehydrogenase promoter, PgpdA or promoters obtained from the genes for Saccharomyces cerevisiae enolase (EN0-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488. Selecting a suitable promoter for expression in yeast is well know and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

Useful terminators for fungal host cells can be obtained from the genes encoding Aspergillus nidulans anthranilate synthase, Aspergillus niger glucoamylase, Aspergillus niger α-glucosidase, Aspergillus oryzae TAKA amylase, and Fusarium oxysporum trypsin-like protease; while useful terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Useful leaders for fungal host cells can be obtained from the genes for Aspergillus oryzae TAKA amylase and Aspergillus nidulans triose phosphate isomerase, while useful leaders for yeast host cells can be obtained from the genes for Saccharomyces cerevisiae enolase (EN0-1), Saccharomyces cerevisiae 3-phosphoglycerate kinase, Saccharomyces cerevisiae α-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used. Useful polyadenylation sequences for fungal host cells can be obtained from the genes for Aspergillus nidulans anthranilate synthase, Aspergillus niger glucoamylase, Aspergillus niger α-glucosidase Aspergillus oryzae TAKA amylase, and Fusarium oxysporum trypsin-like protease; while useful polyadenylation sequences for yeast host cells are described by Guo and Sherman, 1995, Mol. Cellular Biol. 15: 5983-5990.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. In fungi, the Aspergillus niger glucoamylase promoter, Aspergillus oryzae TAKA α-amylase promoter, and Aspergillus oryzae glucoamylase promoter may be used; while in yeast, the ADH2 system or GAL 1 system may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

Various nucleotide sequences in addition to the polynucleotide construct of the invention may be joined together to produce a recombinant expression vector, which may include one or more convenient restriction sites to allow for insertion or substitution of the polynucleotide sequence encoding the P450 of the invention at such sites. The recombinant expression vector may be any vector (e.g., a plasmid or virus or chromosomal) that can be conveniently subjected to recombinant DNA procedures and can bring about expression of the P450 encoding polynucleotide. The choice of the vector will typically depend on the compatibility of the vector with the host cell into which the vector is to be introduced. The vector may be an autonomously replicating vector, i.e., a vector that exists as an extrachromosomal entity, the replication of which is independent of chromosomal replication, e.g., a plasmid (linear or closed circular plasmid), an extrachromosomal element, a minichromosome, or an artificial chromosome. The vector may contain any means for assuring self-replication. Alternatively, the vector may, when introduced into the host cell, integrate into the genome and replicate together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like. Useful selectable markers for fungal host cells include amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Useful selectable markers for yeast host cells include, but are not limited to, ADE2, HIS3, LEU2, LYS2, MET3, TRP1, and URA3.

The vector may further contain element(s) that permits integration of the vector into genome of the host cell or permits autonomous replication of the vector in the cell independent of the genome. For integration into the host cell genome, the vector may rely on the polynucleotide encoding the P450 or any other element of the vector for integration into the genome by homologous or non-homologous recombination. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” refers to a polynucleotide that enables a plasmid or vector to replicate in vivo. Useful origins of replication for fungal cells include AMA 1 and ANSI. (Gems et al., 1991, Gene 98: 61-67; Cullen et al., 1987, Nucleic Acids Res. 15: 9163-9175; WO 00/24883). Isolation of the AMA 1 sequence and construction of plasmids or vectors comprising the gene can be accomplished using the methods disclosed in WO2000/24883. Useful origins of replication for yeast host cells are the 2-micron origin of replication, ARS1, ARS4, the combination of ARS1 and CEN3, and the combination of ARS4 and CEN6.

As mentioned, supra, more than one copy of a polynucleotide encoding the UGT or pathway polypeptide of the invention may be inserted into a host cell to increase production of the UGT or pathway polypeptide. An increase in the copy number can be obtained by integrating one or more additional copies of the enzyme coding sequence into the host cell genome or by including an amplifiable selectable marker gene with the polynucleotide, so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present disclosure are well known to one skilled in the art (see, e.g., Green & Sambrook, 2012, Molecular cloning: A laboratory Manual, Fourth Edition, Cold Spring Harbor Laboratory, New York, USA).

In alignment with the above also disclosed herein is microbial host cell comprising the polynucleotide construct as described, supra.

### Cell Cultures

The invention also provides a cell culture, comprising any host cell of the invention and a growth medium. Suitable growth mediums for host cells such as mammalian, insect, plant, fungal and/or yeast cells are known in the art.

### Fermentation Methods

Where the method of the invention is wholly or partially performed by fermenting a cell culture of the the genetically modified host cell of the invention, the method claims suitably further comprise:
a) culturing the cell culture of invention at conditions allowing the host cell to produce the oripavine glycoside and/or nororipavine glycoside;
b) optionally deglycosylating the oripavine glycoside and/or nororipavine glycoside into an oripavine aglycone and/or nororipavine aglycone; and
c) optionally recovering and/or isolating the oripavine glycoside and/or nororipavine glycoside and/or the oripavine aglycone and/or nororipavine aglycone.

The cell culture can be cultivated in a nutrient medium and at conditions suitable for production of the oripavine glycoside and/or nororipavine glycoside of the invention and/or propagating cell count using methods known in the art. For example, the culture may be cultivated by shake flask cultivation, or small-scale or large-scale fermentation (including continuous, batch, fed-batch, feed and draw, or solid-state fermentations) in laboratory or industrial fermentors in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium comprises a carbon source (e.g. glucose, maltose, molasses, starch, cellulose, xylan, pectin, lignocellolytic biomass hydrolysate, etc.), a nitrogen source (e. g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

The cultivation of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions for yeast and filamentous fungi are a temperature in the range of from about 25° C. to about 55° C. and at a pH of from about 3 to about 9. The appropriate conditions are usually selected based on the choice of host cell. Accordingly, in an embodiment the method of the invention further comprises one or more elements selected from:
a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days;
g) culturing the cell culture under fed-batch, repeated fed-batch, continuous, or semi-continuous conditions; and
h) culturing the cell culture in the presence of an organic solvent to improve the solubility of metabolites of the benzylisoquinoline alkaloid pathway.

The fermentation method of the invention, further suitably comprise feeding one or more exogenous oripavine acceptor and/or nororipavine acceptor or precursors thereof and/or glycoside donors to the cell culture.

It has been found that in vivo demethylation of thebaine into oripavine, thebaine into northebaine, northebaine into nororipavine, and/or oripavine into nororipavine the conversion rates of the demethylases or other pathway enzymes employed are positively affected by glycosylating the produced oripavine and/or nororipavine. Accordingly, in a special embodiment the fermentation method includes host cell which expresses one or more demethylases and/or other pathway enzymes involved in production or demethylating of thebaine into oripavine and/or nororipavine, and wherein the conversion rates (units mass⁻¹ time⁻¹) of the one or more demethylases and/or other pathway enzymes are increased compared to the conversion rates in a host cell not expressing the UGTs.

The cell culture and/or the the metabolites comprised therein may be recovered and or isolated using methods known in the art. For example, the cells and or metabolites may be recovered from the nutrient medium by conventional procedures including, but not limited to, centrifugation, filtration, spray-drying, or lyophilization. In a particular embodiment the method includes a recovery and/or isolation step comprising separating a liquid phase of the cell or cell culture from a solid phase of the cell or cell culture to obtain a supernatant comprising the oripavine glycoside and/or nororipavine glycoside and subjecting the supernatant to one or more steps selected from:
a) disrupting the host cell to release intracellular oripavine and/or nororipavine and/or oripavine glycoside and/or nororipavine glycoside into the supernatant;
b) separating the supernatant form the solid phase of the host cell, such as by filtration or gravity separation;
c) contacting the supernatant with one or more adsorbent resins in order to obtain at least a portion of the produced oripavine glycoside and/or nororipavine glycoside;
d) contacting the supernatant with one or more ion exchange or reversed-phase chromatography columns in order to obtain at least a portion of the oripavine glycoside and/or nororipavine glycoside;
e) extracting the oripavine, nororipavine, oripavine glycoside and/or nororipavine glycoside; and
f) precipitating the oripavine glycoside and/or nororipavine glycoside by crystallization or evaporating the solvent of the liquid phase; and optionally isolating the oripavine glycoside and/or nororipavine glycoside by filtration or gravity separation;
thereby recovering and/or isolating the oripavine glycoside and/or nororipavine glycoside.

### Fermentation Compositions

The invention further provides a fermentation composition comprising the cell culture of the invention and the oripavine glycosides and/or nororipavine glycosides comprised therein.

In one embodiment at least 10%, 25%, 50%, such as at least 75%, such as at least 95%, such as at least 99% of the cells of the fermentation composition of the invention are lysed. Further in the fermentation composition of the invention at least 10%, 25%, 50%, such as at least 75%, such as at least 95%, such as at least 99% of solid cellular material may have been removed and separated from a liquid phase. Moreover, in addition to oripavine glycosides and/or nororipavine glycosides the fermentation composition of the invention may also comprise precursors, products, metabolites of the benzylisoquionoline alkaloid pathway, in particular oripavine and/or nororipavine or comprise one or more compounds selected from trace metals, vitamins, salts, yeast nitrogen base, carbon source, YNB, and/or amino acids of the fermentation. In particular the fermentation composition comprises a concentration of oripavine glycosides and/or nororipavine glycosides of at least 1 mg/kg composition, such as at least 5 mg/kg, such as at least 10 mg/kg, such as at least 20 mg/kg, such as at least 50 mg/kg, such as at least 100 mg/kg, such as at least 500 mg/kg, such as at least 1000 mg/kg, such as at least 5000 mg/kg, such as at least 10000 mg/kg, such as at least 50000 mg/kg.

### Compositions

In a further aspect the invention further provides a composition comprising the fermentation composition of the invention and one or more carriers, agents, additives and/or excipients. Carriers, agents, additives and/or excipients includes formulation additives, stabilising agent, fillers and the like. The composition may be formulated into a dry solid form by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

### Sequences

The present application contains a Sequence Listing prepared in Patent In version 3.5.1, which is also submitted electronically in ST25 format which is hereby incorporated by reference in its entirety. For further reference, the following sequences are included with this application:

| SEQ ID NO: 1 | DNA | sequence | Glycosyl transferase | AHX74090 | From | Eucommia |
|---|---|---|---|---|---|---|
| | | of | | | | ulmoides |
| SEQ ID NO: 2 | DNA | sequence | Glycosyl transferase | AKA44592 | From | Panax ginseng |
| | | of | | | | |
| SEQ ID NO: 3 | DNA | sequence | Glycosyl transferase | AOC55048 | From | Carthamus |
| | | of | | | | tinctorius |
| SEQ ID NO: 4 | DNA | sequence | Glycosyl transferase | AUM57504 | From | Camellia |
| | | of | | | | fraterna |
| SEQ ID NO: 5 | DNA | sequence | Glycosyl transferase | BAI65911 | From | Forsythia x |
| | | of | | | | intermedia |
| SEQ ID NO: 6 | DNA | sequence | Glycosyl transferase | GEV22806 | From | Tanacetum |
| | | of | | | | cinerariifolium |
| SEQ ID NO: 7 | DNA | sequence | Glycosyl transferase | GEZ13352 | From | Tanacetum |
| | | of | | | | cinerariifolium |
| SEQ ID NO: 8 | DNA | sequence | Glycosyl transferase | KAA8526165 | From | Nyssa sinensis |
| | | of | | | | |
| SEQ ID NO: 9 | DNA | sequence | Glycosyl transferase | KAD3640082 | From | Mikania |
| | | of | | | | micrantha |
| SEQ ID NO: | DNA | sequence | Glycosyl transferase | KAD3640082_Scopt | From | Artificial |
| 10 | | of | | | | |
| SEQ ID NO: | DNA | sequence | Glycosyl transferase | KAD3640083 | From | Mikania |
| 11 | | of | | | | micrantha |
| SEQ ID NO: | DNA | sequence | Glycosyl transferase | KAD3640083_Scopt | From | Artificial |
| 12 | | of | | | | |
| SEQ ID NO: | DNA | sequence | Glycosyl transferase | KAD3640084 | From | Mikania |
| 13 | | of | | | | micrantha |
| SEQ ID NO: | DNA | sequence | Glycosyl transferase | KAD3640084_Scopt | From | Artificial |
| 14 | | of | | | | |
| SEQ ID NO: | DNA | sequence | Glycosyl transferase | KAD3640181 | From | Mikania |
| 15 | | of | | | | micrantha |
| SEQ ID NO: | DNA | sequence | Glycosyl transferase | KAD3640181_Scopt | From | Artificial |
| 16 | | of | | | | |
| SEQ ID NO: | DNA | sequence | Glycosyl transferase | PWA70520 | From | Artemisia |
| 17 | | of | | | | annua |
| SEQ ID NO: | DNA | sequence | Glycosyl transferase | PWA74166 | From | Artemisia |
| 18 | | of | | | | annua |
| SEQ ID NO: | DNA | sequence | Glycosyl transferase | PWA79737 | From | Artemisia |
| 19 | | of | | | | annua |
| SEQ ID NO: | DNA | sequence | Glycosyl transferase | PWA89810 | From | Artemisia |
| 20 | | of | | | | annua |
| SEQ ID NO: | DNA | sequence | Glycosyl transferase | SrUGT71E1 | From | Stevia |
| 21 | | of | | | | rebaudiana |
| SEQ ID NO: | DNA | sequence | Glycosyl transferase | SrUGT71E1_opt | From | Artificial |
| 22 | | of | | | | |
| SEQ ID NO: | DNA | sequence | Glycosyl transferase | XP_021998812 | From | Helianthus |
| 23 | | of | | | | annuus |
| SEQ ID NO: | DNA | sequence | Glycosyl transferase | XP_021998812_ | From | Artificial |
| 24 | | of | | Scopt | | |
| SEQ ID NO: | DNA | sequence | Glycosyl transferase | XP_023770763 | From | Lactuca sativa |
| 25 | | of | | | | |
| SEQ ID NO: | DNA | sequence | Glycosyl transferase | XP_023770764 | From | Lactuca sativa |
| 26 | | of | | | | |
| SEQ ID NO: | DNA | sequence | Glycosyl transferase | XP_023770764_ | From | Artificial |
| 27 | | of | | Scopt | | |
| SEQ ID NO: | DNA | sequence | Glycosyl transferase | XP_027172778 | From | Sesamum |
| 28 | | of | | | | indicum |
| SEQ ID NO: | DNA | sequence | Glycosyl transferase | XP_028099977 | From | Camellia |
| 29 | | of | | | | sinensis |
| SEQ ID NO: | DNA | sequence | Glycosyl transferase | XP_028101314 | From | Camellia |
| 30 | | of | | | | sinensis |
| SEQ ID NO: | DNA | sequence | Glycosyl transferase | KAB1219588 | From | Morella rubra |
| 31 | | of | | | | |
| SEQ ID NO: | DNA | sequence | Glycosyl transferase | KAF3968553 | From | Castanea |
| 32 | | of | | | | mollissima |
| SEQ ID NO: | DNA | sequence | Glycosyl transferase | KAF3968553_Scopt | From | Artificial |
| 33 | | of | | | | |
| SEQ ID NO: | DNA | sequence | Glycosyl transferase | KAF3968554 | From | Castanea |
| 34 | | of | | | | mollissima |
| SEQ ID NO: | DNA | sequence | Glycosyl transferase | KAF3968554_Scopt | From | Artificial |
| 35 | | of | | | | |
| SEQ ID NO: | DNA | sequence | Glycosyl transferase | Qs72S_1 | From | Quercus suber |
| 36 | | of | | | | |
| SEQ ID NO: | DNA | sequence | Glycosyl transferase | Qs72S_1_opt | From | Artificial |
| 37 | | of | | | | |
| SEQ ID NO: | DNA | sequence | Glycosyl transferase | XP_023875154 | From | Quercus suber |
| 38 | | of | | | | |
| SEQ ID NO: | DNA | sequence | Glycosyl transferase | XP_023875154_ | From | Artificial |
| 39 | | of | | Scopt | | |
| SEQ ID NO: | DNA | sequence | Glycosyl transferase | XP_023876189 | From | Quercus suber |
| 40 | | of | | | | |
| SEQ ID NO: | DNA | sequence | Glycosyl transferase | XP_023876282 | From | Quercus suber |
| 41 | | of | | | | |
| SEQ ID NO: | DNA | sequence | Glycosyl transferase | XP_023905565 | From | Quercus suber |
| 42 | | of | | | | |
| SEQ ID NO: | DNA | sequence | Glycosyl transferase | XP_023905565_ | From | Artificial |
| 43 | | of | | Scopt | | |
| SEQ ID NO: | DNA | sequence | Glycosyl transferase | XP_023914549 | From | Quercus suber |
| 44 | | of | | | | |
| SEQ ID NO: | DNA | sequence | Glycosyl transferase | XP_023914549_ | From | Artificial |
| 45 | | of | | Scopt | | |
| SEQ ID NO: | DNA | sequence | Glycosyl transferase | XP_023923919 | From | Quercus suber |
| 46 | | of | | | | |
| SEQ ID NO: | DNA | sequence | Glycosyl transferase | XP_030967178 | From | Quercus lobata |
| 47 | | of | | | | |
| SEQ ID NO: | DNA | sequence | Glycosyl transferase | XP_030967178_ | From | Artificial |
| 48 | | of | | Scopt | | |
| SEQ ID NO: | DNA | sequence | Glycosyl transferase | GEU38196 | From | Tanacetum |
| 49 | | of | | | | cinerariifolium |
| SEQ ID NO: | DNA | sequence | Glycosyl transferase | GEU38196_Scopt | From | Artificial |
| 50 | | of | | | | |
| SEQ ID NO: | DNA | sequence | Glycosyl transferase | KAD4384427 | From | Mikania |
| 51 | | of | | | | micrantha |
| SEQ ID NO: | DNA | sequence | Glycosyl transferase | KAD4384427_Scopt | From | Artificial |
| 52 | | of | | | | |
| SEQ ID NO: | DNA | sequence | Glycosyl transferase | PWA36889 | From | Artemisia |
| 53 | | of | | | | annua |
| SEQ ID NO: | DNA | sequence | Glycosyl transferase | PWA36889_Scopt | From | Artificial |
| 54 | | of | | | | |
| SEQ ID NO: | DNA | sequence | Glycosyl transferase | PWA37695 | From | Artemisia |
| 55 | | of | | | | annua |
| SEQ ID NO: | DNA | sequence | Glycosyl transferase | SrUGT73E1 | From | Stevia |
| 56 | | of | | | | rebaudiana |
| SEQ ID NO: | DNA | sequence | Glycosyl transferase | SrUGT73E1_opt | From | Artificial |
| 57 | | of | | | | |
| SEQ ID NO: | DNA | sequence | Glycosyl transferase | XP_023746016 | From | Lactuca sativa |
| 58 | | of | | | | |
| SEQ ID NO: | DNA | sequence | Glycosyl transferase | XP_023746016_ | From | Artificial |
| 59 | | of | | Scopt | | |
| SEQ ID NO: | DNA | sequence | Glycosyl transferase | XP_023764656 | From | Lactuca sativa |
| 60 | | of | | | | |
| SEQ ID NO: | Protein | sequence | Glycosyl transferase | AHX74090 | From | Eucommia |
| 61 | | of | | | | ulmoides |
| SEQ ID NO: | Protein | sequence | Glycosyl transferase | AKA44592 | From | Panax ginseng |
| 62 | | of | | | | |
| SEQ ID NO: | Protein | sequence | Glycosyl transferase | AOC55048 | From | Carthamus |
| 63 | | of | | | | tinctorius |
| SEQ ID NO: | Protein | sequence | Glycosyl transferase | AUM57504 | From | Camellia |
| 64 | | of | | | | fraterna |
| SEQ ID NO: | Protein | sequence | Glycosyl transferase | BAI65911 | From | Forsythia x |
| 65 | | of | | | | intermedia |
| SEQ ID NO: | Protein | sequence | Glycosyl transferase | GEV22806 | From | Tanacetum |
| 66 | | of | | | | cinerariifolium |
| SEQ ID NO: | Protein | sequence | Glycosyl transferase | GEZ13352 | From | Tanacetum |
| 67 | | of | | | | cinerariifolium |
| SEQ ID NO: | Protein | sequence | Glycosyl transferase | KAA8526165 | From | Nyssa sinensis |
| 68 | | of | | | | |
| SEQ ID NO: | Protein | sequence | Glycosyl transferase | KAD3640082 | From | Mikania |
| 69 | | of | | | | micrantha |
| SEQ ID NO: | protein | sequence | Glycosyl transferase | KAD3640082_Scopt | From | Mikania |
| 70 | | of | | | | micrantha |
| SEQ ID NO: | Protein | sequence | Glycosyl transferase | KAD3640083 | From | Mikania |
| 71 | | of | | | | micrantha |
| SEQ ID NO: | protein | sequence | Glycosyl transferase | KAD3640083_Scopt | From | Mikania |
| 72 | | of | | | | micrantha |
| SEQ ID NO: | Protein | sequence | Glycosyl transferase | KAD3640084 | From | Mikania |
| 73 | | of | | | | micrantha |
| SEQ ID NO: | protein | sequence | Glycosyl transferase | KAD3640084_Scopt | From | Mikania |
| 74 | | of | | | | micrantha |
| SEQ ID NO: | Protein | sequence | Glycosyl transferase | KAD3640181 | From | Mikania |
| 75 | | of | | | | micrantha |
| SEQ ID NO: | protein | sequence | Glycosyl transferase | KAD3640181_Scopt | From | Mikania |
| 76 | | of | | | | micrantha |
| SEQ ID NO: | Protein | sequence | Glycosyl transferase | PWA70520 | From | Artemisia |
| 77 | | of | | | | annua |
| SEQ ID NO: | Protein | sequence | Glycosyl transferase | PWA74166 | From | Artemisia |
| 78 | | of | | | | annua |
| SEQ ID NO: | Protein | sequence | Glycosyl transferase | PWA79737 | From | Artemisia |
| 79 | | of | | | | annua |
| SEQ ID NO: | Protein | sequence | Glycosyl transferase | PWA89810 | From | Artemisia |
| 80 | | of | | | | annua |
| SEQ ID NO: | Protein | sequence | Glycosyl transferase | SrUGT71E1 | From | Stevia |
| 81 | | of | | | | rebaudiana |
| SEQ ID NO: | protein | sequence | Glycosyl transferase | SrUGT71E1_opt | From | Stevia |
| 82 | | of | | | | rebaudiana |
| SEQ ID NO: | Protein | sequence | Glycosyl transferase | XP_021998812 | From | Helianthus |
| 83 | | of | | | | annuus |
| SEQ ID NO: | protein | sequence | Glycosyl transferase | XP_021998812_ | From | Helianthus |
| 84 | | of | | Scopt | | annuus |
| SEQ ID NO: | Protein | sequence | Glycosyl transferase | XP_023770763 | From | Lactuca sativa |
| 85 | | of | | | | |
| SEQ ID NO: | Protein | sequence | Glycosyl transferase | XP_023770764 | From | Lactuca sativa |
| 86 | | of | | | | |
| SEQ ID NO: | protein | sequence | Glycosyl transferase | XP_023770764_ | From | Lactuca sativa |
| 87 | | of | | Scopt | | |
| SEQ ID NO: | Protein | sequence | Glycosyl transferase | XP_027172778 | From | Sesamum |
| 88 | | of | | | | indicum |
| SEQ ID NO: | Protein | sequence | Glycosyl transferase | XP_028099977 | From | Camellia |
| 89 | | of | | | | sinensis |
| SEQ ID NO: | Protein | sequence | Glycosyl transferase | XP_028101314 | From | Camellia |
| 90 | | of | | | | sinensis |
| SEQ ID NO: | Protein | sequence | Glycosyl transferase | KAB1219588 | From | Morella rubra |
| 91 | | of | | | | |
| SEQ ID NO: | Protein | sequence | Glycosyl transferase | KAF3968553 | From | Castanea |
| 92 | | of | | | | mollissima |
| SEQ ID NO: | protein | sequence | Glycosyl transferase | KAF3968553_Scopt | From | Castanea |
| 93 | | of | | | | mollissima |
| SEQ ID NO: | Protein | sequence | Glycosyl transferase | KAF3968554 | From | Castanea |
| 94 | | of | | | | mollissima |
| SEQ ID NO: | protein | sequence | Glycosyl transferase | KAF3968554_Scopt | From | Castanea |
| 95 | | of | | | | mollissima |
| SEQ ID NO: | Protein | sequence | Glycosyl transferase | Qs72S_1 | From | Quercus suber |
| 96 | | of | | | | |
| SEQ ID NO: | protein | sequence | Glycosyl transferase | Qs72S_1_opt | From | Quercus suber |
| 97 | | of | | | | |
| SEQ ID NO: | Protein | sequence | Glycosyl transferase | XP_023875154 | From | Quercus suber |
| 98 | | of | | | | |
| SEQ ID NO: | protein | sequence | Glycosyl transferase | XP_023875154_ | From | Quercus suber |
| 99 | | of | | Scopt | | |
| SEQ ID NO: | Protein | sequence | Glycosyl transferase | XP_023876189 | From | Quercus suber |
| 100 | | of | | | | |
| SEQ ID NO: | Protein | sequence | Glycosyl transferase | XP_023876282 | From | Quercus suber |
| 101 | | of | | | | |
| SEQ ID NO: | Protein | sequence | Glycosyl transferase | XP_023905565 | From | Quercus suber |
| 102 | | of | | | | |
| SEQ ID NO: | protein | sequence | Glycosyl transferase | XP_023905565_ | From | Quercus suber |
| 103 | | of | | Scopt | | |
| SEQ ID NO: | Protein | sequence | Glycosyl transferase | XP_023914549 | From | Quercus suber |
| 104 | | of | | | | |
| SEQ ID NO: | protein | sequence | Glycosyl transferase | XP_023914549_ | From | Quercus suber |
| 105 | | of | | Scopt | | |
| SEQ ID NO: | Protein | sequence | Glycosyl transferase | XP_023923919 | From | Quercus suber |
| 106 | | of | | | | |
| SEQ ID NO: | Protein | sequence | Glycosyl transferase | XP_030967178 | From | Quercus lobata |
| 107 | | of | | | | |
| SEQ ID NO: | protein | sequence | Glycosyl transferase | XP_030967178_ | From | Quercus lobata |
| 108 | | of | | Scopt | | |
| SEQ ID NO: | Protein | sequence | Glycosyl transferase | GEU38196 | From | Tanacetum |
| 109 | | of | | | | cinerariifolium |
| SEQ ID NO: | protein | sequence | Glycosyl transferase | GEU38196_Scopt | From | Tanacetum |
| 110 | | of | | | | cinerariifolium |
| SEQ ID NO: | Protein | sequence | Glycosyl transferase | KAD4384427 | From | Mikania |
| 111 | | of | | | | micrantha |
| SEQ ID NO: | protein | sequence | Glycosyl transferase | KAD4384427_Scopt | From | Mikania |
| 112 | | of | | | | micrantha |
| SEQ ID NO: | Protein | sequence | Glycosyl transferase | PWA36889 | From | Artemisia |
| 113 | | of | | | | annua |
| SEQ ID NO: | protein | sequence | Glycosyl transferase | PWA36889_Scopt | From | Artemisia |
| 114 | | of | | | | annua |
| SEQ ID NO: | Protein | sequence | Glycosyl transferase | PWA37695 | From | Artemisia |
| 115 | | of | | | | annua |
| SEQ ID NO: | Protein | sequence | Glycosyl transferase | SrUGT73E1 | From | Stevia |
| 116 | | of | | | | rebaudiana |
| SEQ ID NO: | protein | sequence | Glycosyl transferase | SrUGT73E1_opt | From | Stevia |
| 117 | | of | | | | rebaudiana |
| SEQ ID NO: | Protein | sequence | Glycosyl transferase | XP_023746016 | From | Lactuca sativa |
| 118 | | of | | | | |
| SEQ ID NO: | protein | sequence | Glycosyl transferase | XP_023746016_ | From | Lactuca sativa |
| 119 | | of | | Scopt | | |
| SEQ ID NO: | Protein | sequence | Glycosyl transferase | XP_023764656 | From | Lactuca sativa |
| 120 | | of | | | | |
| SEQ ID NO: | protein | sequence | DAHP | Aro4fbr | From | Artificial |
| 121 | | of | | | | |
| SEQ ID NO: | DNA | sequence | DAHP | Aro4fbr | From | Artificial |
| 122 | | of | | | | |
| SEQ ID NO: | protein | sequence | chorismate mutase | ARO7fbr | From | Artificial |
| 123 | | of | | | | |
| SEQ ID NO: | DNA | sequence | chorismate mutase | ARO7fbr | From | Artificial |
| 124 | | of | | | | |
| SEQ ID NO: | protein | sequence | Tyr1 | Tyr1 | From | S. cerevisiae |
| 125 | | of | | | | |
| SEQ ID NO: | DNA | sequence | Tyr1 | Tyr1 | From | S. cerevisiae |
| 126 | | of | | | | |
| SEQ ID NO: | protein | sequence | TH | SoCYP76ADr9 | From | Spinacia |
| 127 | | of | | | | oleracea |
| SEQ ID NO: | DNA | sequence | TH | SoCYP76ADr9 | From | Spinacia |
| 128 | | of | | | | oleracea |
| SEQ ID NO: | protein | sequence | CPR′′′ | BvCPR1 | From | Beta vulgaris |
| 129 | | of | | | | |
| SEQ ID NO: | DNA | sequence | CPR′′′ | BvCPR1 | From | Beta vulgaris |
| 130 | | of | | | | |
| SEQ ID NO: | protein | sequence | DoDC | PpDoDC | From | Pseudomonas |
| 131 | | of | | | | putida |
| SEQ ID NO: | DNA | sequence | DoDC | PpDoDC | From | Pseudomonas |
| 132 | | of | | | | putida |
| SEQ ID NO: | protein | sequence | NCS | d19CjNCS | From | Coptis japonica |
| 133 | | of | | | | |
| SEQ ID NO: | DNA | sequence | NCS | d19CjNCS | From | Coptis japonica |
| 134 | | of | | | | |
| SEQ ID NO: | protein | sequence | 6-OMT | Ps6OMT_Q6WUC1 | From | Papaver |
| 135 | | of | | | | somniferum |
| SEQ ID NO: | DNA | sequence | 6-OMT | Ps6OMT_Q6WUC1 | From | Papaver |
| 136 | | of | | | | somniferum |
| SEQ ID NO: | protein | sequence | CNMT | CjCNMT | From | Coptis japonica |
| 137 | | of | | | | |
| SEQ ID NO: | DNA | sequence | CNMT | CjCNMT | From | Coptis japonica |
| 138 | | of | | | | |
| SEQ ID NO: | protein | sequence | NMCH | EcNMCH | From | Eschscholzia |
| 139 | | of | | | | californica |
| SEQ ID NO: | DNA | sequence | NMCH | EcNMCH | From | Eschscholzia |
| 140 | | of | | | | californica |
| SEQ ID NO: | protein | sequence | 4′-OMT | Cj4OMT | From | Coptis japonica |
| 141 | | of | | | | |
| SEQ ID NO: | DNA | sequence | 4′-OMT | Cj4OMT | From | Coptis japonica |
| 142 | | of | | | | |
| SEQ ID NO: | protein | sequence | DRS-DRR | DRS-DRR | From | Papaver |
| 143 | | of | | | | bracteatum |
| SEQ ID NO: | DNA | sequence | DRS-DRR | DRS-DRR | From | Papaver |
| 144 | | of | | | | bracteatum |
| SEQ ID NO: | protein | sequence | SAS | PbSAS | From | Papaver |
| 145 | | of | | | | bracteatum |
| SEQ ID NO: | DNA | sequence | SAS | PbSAS | From | Papaver |
| 146 | | of | | | | bracteatum |
| SEQ ID NO: | protein | sequence | SAT | PsSAT | From | Papaver |
| 147 | | of | | | | somniferum |
| SEQ ID NO: | DNA | sequence | SAT | PsSAT | From | Papaver |
| 148 | | of | | | | somniferum |
| SEQ ID NO: | protein | sequence | SAR | pbSalR | From | Papaver |
| 149 | | of | | | | bracteatum |
| SEQ ID NO: | DNA | sequence | SAR | pbSalR | From | Papaver |
| 150 | | of | | | | bracteatum |
| SEQ ID NO: | protein | sequence | THS | PsTHS1 | From | Papaver |
| 151 | | of | | | | somniferum |
| SEQ ID NO: | DNA | sequence | THS | PsTHS1 | From | Papaver |
| 152 | | of | | | | somniferum |
| SEQ ID NO: | protein | sequence | N-demethylase | Hv_CYP_ | From | Heliothis |
| 153 | | of | | A0A2A4JAM9 | | virescens |
| SEQ ID NO: | DNA | sequence | N-demethylase | Hv_CYP_ | From | Heliothis |
| 154 | | of | | A0A2A4JAM9 | | virescens |
| SEQ ID NO: | protein | sequence | (O- and N | HaCYP6AE15v2 | From | Helicoverpa |
| 155 | | of | demethylase | | | armigera |
| SEQ ID NO: | DNA | sequence | (O- and N | HaCYP6AE15v2 | From | Helicoverpa |
| 156 | | of | demethylase | | | armigera |
| SEQ ID NO: | protein | sequence | O- and N- | CYPDN_39 | From | Rhizopus |
| 157 | | of | demethylase | | | microsporus |
| SEQ ID NO: | DNA | sequence | O- and N- | CYPDN_39 | From | Rhizopus |
| 158 | | of | demethylase | | | microsporus |
| SEQ ID NO: | protein | sequence | CPR′ | HaCPR_E0A3A7 | From | Helicoverpa |
| 159 | | of | | | | armigera |
| SEQ ID NO: | DNA | sequence | CPR′ | HaCPR_E0A3A7 | From | Helicoverpa |
| 160 | | of | | | | armigera |
| SEQ ID NO: | protein | sequence | CPR′ | CeCPR | From | Cunninghamella |
| 161 | | of | | | | elegans |
| SEQ ID NO: | DNA | sequence | CPR′ | CeCPR | From | Cunninghamella |
| 162 | | of | | | | elegans |
| SEQ ID NO: | protein | sequence | Transporter MDR1 | T1_CjaMDR1_GA | From | Camellia |
| 163 | | of | | | | japonica |
| SEQ ID NO: | DNA | sequence | Transporter MDR1 | T1_CjaMDR1_GA | From | Camellia |
| 164 | | of | | | | japonica |
| SEQ ID NO: | protein | sequence | KO ARI1 | ARI1 | From | Saccharomyces |
| 165 | | of | | | | cerevisiae |
| SEQ ID NO: | DNA | sequence | KO ARI1 | ARI1 | From | Saccharomyces |
| 166 | | of | | | | cerevisiae |
| SEQ ID NO: | protein | sequence | KO GRE2 | GRE2 | From | Saccharomyces |
| 167 | | of | | | | cerevisiae |
| SEQ ID NO: | DNA | sequence | KO GRE2 | GRE2 | From | Saccharomyces |
| 168 | | of | | | | cerevisiae |
| SEQ ID NO: | DNA | sequence | Glycosyl transferase | | From | artificial |
| 169 | | of | | | | |
| SEQ ID NO: | Protein | sequence | Glycosyl transferase | | From | artificial |
| 170 | | of | | | | |
| SEQ ID NO: | DNA | sequence | Glycosyl transferase | | From | artificial |
| 171 | | of | | | | |
| SEQ ID NO: | Protein | sequence | Glycosyl transferase | | From | artificial |
| 172 | | of | | | | |
| SEQ ID NO: | DNA | sequence | Glycosyl transferase | | From | artificial |
| 173 | | of | | | | |
| SEQ ID NO: | Protein | sequence | Glycosyl transferase | | From | artificial |
| 174 | | of | | | | |
| SEQ ID NO: | DNA | sequence | Glycosyl transferase | | From | artificial |
| 175 | | of | | | | |
| SEQ ID NO: | Protein | sequence | Glycosyl transferase | | From | artificial |
| 176 | | of | | | | |
| SEQ ID NO: | DNA | sequence | Glycosyl transferase | | From | artificial |
| 177 | | of | | | | |
| SEQ ID NO: | Protein | sequence | Glycosyl transferase | | From | artificial |
| 178 | | of | | | | |
| SEQ ID NO: | DNA | sequence | Glycosyl transferase | | From | artificial |
| 179 | | of | | | | |
| SEQ ID NO: | Protein | sequence | Glycosyl transferase | | From | artificial |
| 180 | | of | | | | |
| SEQ ID NO: | DNA | sequence | Glycosyl transferase | | From | artificial |
| 181 | | of | | | | |
| SEQ ID NO: | Protein | sequence | Glycosyl transferase | | From | artificial |
| 182 | | of | | | | |
| SEQ ID NO: | DNA | sequence | Glycosyl transferase | | From | artificial |
| 183 | | of | | | | |
| SEQ ID NO: | Protein | sequence | Glycosyl transferase | | From | artificial |
| 184 | | of | | | | |
| SEQ ID NO: | DNA | sequence | Glycosyl transferase | | From | artificial |
| 185 | | of | | | | |
| SEQ ID NO: | Protein | sequence | Glycosyl transferase | | From | artificial |
| 186 | | of | | | | |
| SEQ ID NO: | DNA | sequence | Glycosyl transferase | | From | artificial |
| 187 | | of | | | | |
| SEQ ID NO: | Protein | sequence | Glycosyl transferase | | From | artificial |
| 188 | | of | | | | |
| SEQ ID NO: | DNA | sequence | Glycosyl transferase | | From | artificial |
| 189 | | of | | | | |
| SEQ ID NO: | Protein | sequence | Glycosyl transferase | | From | artificial |
| 190 | | of | | | | |
| SEQ ID NO: | DNA | sequence | Glycosyl transferase | | From | artificial |
| 191 | | of | | | | |
| SEQ ID NO: | Protein | sequence | Glycosyl transferase | | From | artificial |
| 192 | | of | | | | |
| SEQ ID NO: | DNA | sequence | Glycosyl transferase | | From | artificial |
| 193 | | of | | | | |
| SEQ ID NO: | Protein | sequence | Glycosyl transferase | | From | artificial |
| 194 | | of | | | | |
| SEQ ID NO: | DNA | sequence | Glycosyl transferase | | From | artificial |
| 195 | | of | | | | |
| SEQ ID NO: | Protein | sequence | Glycosyl transferase | | From | artificial |
| 196 | | of | | | | |
| SEQ ID NO: | DNA | sequence | Glycosyl transferase | | From | artificial |
| 197 | | of | | | | |
| SEQ ID NO: | Protein | sequence | Glycosyl transferase | | From | artificial |
| 198 | | of | | | | |
| SEQ ID NO: | DNA | sequence | Glycosyl transferase | | From | artificial |
| 199 | | of | | | | |
| SEQ ID NO: | Protein | sequence | Glycosyl transferase | | From | artificial |
| 200 | | of | | | | |
| SEQ ID NO: | DNA | sequence | Glycosyl transferase | | From | artificial |
| 201 | | of | | | | |
| SEQ ID NO: | Protein | sequence | Glycosyl transferase | | From | artificial |
| 202 | | of | | | | |
| SEQ ID NO: | DNA | sequence | Glycosyl transferase | | From | artificial |
| 203 | | of | | | | |
| SEQ ID NO: | Protein | sequence | Glycosyl transferase | | From | artificial |
| 204 | | of | | | | |
| SEQ ID NO: | DNA | sequence | Glycosyl transferase | | From | artificial |
| 205 | | of | | | | |
| SEQ ID NO: | Protein | sequence | Glycosyl transferase | | From | artificial |
| 206 | | of | | | | |
| SEQ ID NO: | DNA | sequence | Glycosyl transferase | | From | artificial |
| 207 | | of | | | | |
| SEQ ID NO: | Protein | sequence | Glycosyl transferase | | From | artificial |
| 208 | | of | | | | |
| SEQ ID NO: | DNA | sequence | Glycosyl transferase | | From | artificial |
| 209 | | of | | | | |
| SEQ ID NO: | Protein | sequence | Glycosyl transferase | | From | artificial |
| 210 | | of | | | | |
| SEQ ID NO: | DNA | sequence | Glycosyl transferase | | From | artificial |
| 211 | | of | | | | |
| SEQ ID NO: | Protein | sequence | Glycosyl transferase | | From | artificial |
| 212 | | of | | | | |
| SEQ ID NO: | DNA | sequence | Glycosyl transferase | | From | artificial |
| 213 | | of | | | | |
| SEQ ID NO: | Protein | sequence | Glycosyl transferase | | From | artificial |
| 214 | | of | | | | |
| SEQ ID NO: | DNA | sequence | Glycosyl transferase | | From | artificial |
| 215 | | of | | | | |
| SEQ ID NO: | Protein | sequence | Glycosyl transferase | | From | artificial |
| 216 | | of | | | | |
| SEQ ID NO: | DNA | sequence | Glycosyl transferase | | From | artificial |
| 217 | | of | | | | |
| SEQ ID NO: | Protein | sequence | Glycosyl transferase | | From | artificial |
| 218 | | of | | | | |
| SEQ ID NO: | DNA | sequence | Glycosyl transferase | | From | artificial |
| 219 | | of | | | | |
| SEQ ID NO: | Protein | sequence | Glycosyl transferase | | From | artificial |
| 220 | | of | | | | |
| SEQ ID NO: | DNA | sequence | Glycosyl transferase | | From | artificial |
| 221 | | of | | | | |
| SEQ ID NO: | Protein | sequence | Glycosyl transferase | | From | artificial |
| 222 | | of | | | | |
| SEQ ID NO: | DNA | sequence | uptake transporter | | From | artificial |
| 223 | | of | | | | |
| SEQ ID NO: | Protein | sequence | uptake transporter | | From | artificial |
| 224 | | of | | | | |
| SEQ ID NO: | DNA | sequence | uptake transporter | | From | artificial |
| 225 | | of | | | | |
| SEQ ID NO: | Protein | sequence | uptake transporter | | From | artificial |
| 226 | | of | | | | |
| SEQ ID NO: | DNA | sequence | uptake transporter | | From | artificial |
| 227 | | of | | | | |
| SEQ ID NO: | Protein | sequence | uptake transporter | | From | artificial |
| 228 | | of | | | | |
| SEQ ID NO: | DNA | sequence | uptake transporter | | From | artificial |
| 229 | | of | | | | |
| SEQ ID NO: | Protein | sequence | uptake transporter | | From | artificial |
| 230 | | of | | | | |
| SEQ ID NO: | DNA | sequence | uptake transporter | | From | artificial |
| 231 | | of | | | | |
| SEQ ID NO: | Protein | sequence | uptake transporter | | From | artificial |
| 232 | | of | | | | |
| SEQ ID NO: | DNA | sequence | uptake transporter | | From | artificial |
| 233 | | of | | | | |
| SEQ ID NO: | Protein | sequence | uptake transporter | | From | artificial |
| 234 | | of | | | | |
| SEQ ID NO: | DNA | sequence | uptake transporter | | From | artificial |
| 235 | | of | | | | |
| SEQ ID NO: | Protein | sequence | uptake transporter | | From | artificial |
| 236 | | of | | | | |
| SEQ ID NO: | DNA | sequence | uptake transporter | | From | artificial |
| 237 | | of | | | | |
| SEQ ID NO: | Protein | sequence | uptake transporter | | From | artificial |
| 238 | | of | | | | |
| SEQ ID NO: | DNA | sequence | uptake transporter | | From | artificial |
| 239 | | of | | | | |
| SEQ ID NO: | Protein | sequence | uptake transporter | | From | artificial |
| 240 | | of | | | | |
| SEQ ID NO: | DNA | sequence | uptake transporter | | From | artificial |
| 241 | | of | | | | |
| SEQ ID NO: | Protein | sequence | uptake transporter | | From | artificial |
| 242 | | of | | | | |
| SEQ ID NO: | DNA | sequence | uptake transporter | | From | artificial |
| 243 | | of | | | | |
| SEQ ID NO: | Protein | sequence | uptake transporter | | From | artificial |
| 244 | | of | | | | |
| SEQ ID NO: | DNA | sequence | uptake transporter | | From | artificial |
| 245 | | of | | | | |
| SEQ ID NO: | Protein | sequence | uptake transporter | | From | artificial |
| 246 | | of | | | | |
| SEQ ID NO: | DNA | sequence | uptake transporter | | From | artificial |
| 247 | | of | | | | |
| SEQ ID NO: | Protein | sequence | uptake transporter | | From | artificial |
| 248 | | of | | | | |
| SEQ ID NO: | DNA | sequence | uptake transporter | | From | artificial |
| 249 | | of | | | | |
| SEQ ID NO: | Protein | sequence | uptake transporter | | From | artificial |
| 250 | | of | | | | |
| SEQ ID NO: | DNA | sequence | uptake transporter | | From | artificial |
| 251 | | of | | | | |
| SEQ ID NO: | Protein | sequence | uptake transporter | | From | artificial |
| 252 | | of | | | | |
| SEQ ID NO: | DNA | sequence | uptake transporter | | From | artificial |
| 253 | | of | | | | |
| SEQ ID NO: | Protein | sequence | uptake transporter | | From | artificial |
| 254 | | of | | | | |
| SEQ ID NO: | DNA | sequence | N-demethylase | | From | artificial |
| 255 | | of | | | | |
| SEQ ID NO: | Protein | sequence | N-demethylase | | From | artificial |
| 256 | | of | | | | |
| SEQ ID NO: | DNA | sequence | N-demethylase | | From | artificial |
| 257 | | of | | | | |
| SEQ ID NO: | Protein | sequence | N-demethylase | | From | artificial |
| 258 | | of | | | | |
| SEQ ID NO: | DNA | sequence | CPR | | From | artificial |
| 259 | | of | | | | |
| SEQ ID NO: | Protein | sequence | CPR | | From | artificial |
| 260 | | of | | | | |

### EXAMPLES

### Materials and Methods

### Materials

Chemicals used in the examples herein e.g. for buffers and substrates are commercial products of at least reagent grade.

### LB Medium

The LB medium as used through the examples is commonly known in the art.

### LB Medium+Ampicillin

The LB medium+ampicillin as used through the examples is commonly known in the art.

### Expression Medium

An expression medium was prepared according to Table 1:

TABLE 1

| | | |
|---|---|---|
| Expression medium | Amount | Final conc |

| | | | | |
|---|---|---|---|---|
| LB liquid media | 100 | mL | 100 | μg/mL |
| Ampicillin (100 μg/μL stock) | 100 | μL | 0.15 | mM |
| Sterile-filtrated IPTG (1M stock) | 15 | μL | 4.5 | mM |

### Resuspension Medium

A resuspension medium was prepared according to Table 2:

TABLE 2

| Resuspension medium | Amount | | Final conc | |
|---|---|---|---|---|
| Tris-HCl, pH 7.4 (1M stock) | 2.5 | mL | 50 | mM |
| MQ H2O | 47.5 | mL | | |
| PMSF (1M stock in DMSO) | 50 | μL | 0.15 | mM |
| EDTA-free Complete mini, | 2 | tablets | | |
| protease inhibitor tablet (Roche)* | | | | |

### Lysis Solution

A solution for lysing cells was prepared according to Table 3:

TABLE 3

| Lysing Solution | Amount | | Final conc | |
|---|---|---|---|---|
| Denarase (250 U/μL stock) DNase, | 0.6 | μL | 300 | U/mL |
| C-LECta. Art no. 20804-100k) | | | | |
| MgCl₂ (1M stock) | 1.3 | μL | 2.6 | mM |
| CaCl₂ (1M stock) | 0.5 | μL | 1 | mM |
| Lysozyme(10 mg/mL stock). (Sigma L6876) | 10 | μL | 0.05 | mg/mL |
| Total | 12.4 | μL | | |

### Elution Buffer

An elution buffer for protein purification was prepared according to Table 4:

TABLE 4

| | | |
|---|---|---|
| Elution buffer | 0.250 | L |
| Tris-HCl pH 7.4 (1M stock) | 5 | mL |
| Imidazole (1M stock) | 125 | mL |
| MilliQ water | 120 | mL |

### SC-his-Leu-Ura Medium

The SC-His-Leu-Ura medium as used through the examples is commonly known in the art.

DELFT Minimal Medium with Oripavine

The DELFT minimal medium as used through the examples is commonly known in the art.

### Seed-Train Medium

A seed train medium was prepared as an aqueous solution of 10.0 g/L succinic acid, 6.0 g/L NaOH, 5.0 g/L (NH₄)₂SO₄, 3.0 g/L KH₂PO₄, 0.5 g/L MgSO₄0.7H₂O, 22 g/L (2%) glucose monohydrate. The aqueous solution was further added 10.0 mL/L of trace metal stock solution (adapted from Hoek et al., 2000) and 12 ml/L of (Delft) vitamin stock solution (Hoek et al., 2000) and the pH of the medium was adjusted to 6.5 prior to sterilization. The medium was sterilized at 121° C. for 20 min before use.

### Batch Medium

A batch medium was prepared as an aqueous solution of 5.0 g/L (NH₄)₂SO₄, 3.0 g/L KH₂PO₄, 0.5 g/L MgSO₄·7H₂O, 1.0 g/L SB2020 (antifoam), 13.0 g/L (1%) glucose monohydrate. The aqueous solution was further added 10 mL/L of trace metal stock solution (adapted from Hoek et al., 2000) and 12 ml/L of (Delft) vitamin stock solution (Hoek et al., 2000). The medium was sterilized at 121° C. for 20 min before use.

### Fed-Batch Medium

A fed-batch medium was prepared as an aqueous solution of 5.0 g/L (NH₄)₂SO₄, 11.2 g/L KH₂PO₄, 6.3 g/L MgSO₄·7H₂O, 4.3 g/L K₂SO₄, 0.347 Na₂SO₄, 1.0 SB2020 (antifoam), 682 (62%) glucose monohydrate. The aqueous solution was further added 14.4 mL/L of trace metal stock solution (adapted from Hoek et al., 2000) and 14.8 ml/L of (Delft) vitamin stock solution (Hoek et al., 2000). The medium was sterilized at 121° C. for 20 min before use.

### Strains

A commercially available E. coli background BL21 (DE3) strain commonly known in the art was used.

Saccharomyces cerevisiae yeast strain sOD157 (MATa his3Δ0 leu2Δ0 ura3Δ0 CATS-91Met GAL2 ho MIP1-661Thr SAL1-1) was used as background strain. Strain sOD157 with the said genotype corresponds to strain S288C (genotype MATa his300 leu200 ura300) which is a publicly available widely used laboratory strain (see the Saccharomyces Genome Database (SGD)). Accordingly, similar results can be reached by using strain S288C as the results demonstrated below using strain sOD157 as the base strain for modification, background and/or control. All strain transformations with relevant plasmids were done using the lithium acetate method (Gietz et al. 2007).

### Genes and Enzymes

Glycosyltransferases (UGTs) from Stevia rebaudiana (subfamily 71 UGT71E1 and subfamily 73 UGT73E1) and from Quercus suber (subfamily 72 Qs72S_1 (XP_023905554)) and sequence homologs thereof according to Table 5 were prepared as ORFS codon optimized for E. coli cloned in the SpeI-XhoI site of vector pRSGLY. pRSGLY is a typical E. coli expression vector comprising ampicillin (AmpR) selection marker, LacO operator sequence for repressor binding, His tag (6×His N-terminal), Rop protein gene, Origin of replication (pBR322/pMB1 ori) and T7 promoter/terminator for directing IPTG inducible expression of the target gene and maintained at medium copy number. Insertion of genes into this vector made expression in E. coli and subsequent purification possible.

TABLE 5

| Subfamily 71 UGT | | Subfamily 72 UGT | | Subfamily 73 UGT | |
|---|---|---|---|---|---|
| Gene | Sequence | Gene | Sequence | Gene | Sequence |
| AHX74090 | SEQ ID NO: 1 | UGT72-0 | — | GEU38196 | SEQ ID NO: 49 |
| AKA44592 | SEQ ID NO: 2 | KAB1219588 | SEQ ID NO: 31 | GEU38196_Scopt | SEQ ID NO: 50 |
| AOC55048 | SEQ ID NO: 3 | UGT72-1 | — | UGT73-1 | — |
| AUM57504 | SEQ ID NO: 4 | UGT72-2 | — | UGT73-2 | — |
| BAI65911 | SEQ ID NO: 5 | KAF3968553 | SEQ ID NO: 32 | KAD4384427 | SEQ ID NO: 51 |
| 71UGT-1 | — | KAF3968553_Scopt | SEQ ID NO: 33 | KAD4384427_Scopt | SEQ ID NO: 52 |
| GEV22806 | SEQ ID NO: 6 | KAF3968554 | SEQ ID NO: 34 | UGT73-3 | — |
| GEZ13352 | SEQ ID NO: 7 | KAF3968554_Scopt | SEQ ID NO: 35 | UGT73-4 | — |
| KAA8526165 | SEQ ID NO: 8 | UGT72-3 | — | PWA36889 | SEQ ID NO: 53 |
| 71UGT-2 | — | UGT72-4 | — | PWA36889_Scopt | SEQ ID NO: 54 |
| 71UGT-3 | — | UGT72-5 | — | UGT73-5 | |
| 71UGT-4 | — | UGT72-6 | — | PWA37695 | SEQ ID NO: 55 |
| KAD3640082 | SEQ ID NO: 9 | Qs72S_1 | SEQ ID NO: 36 | UGT73-6 | — |
| KAD3640082_Scopt | SEQ ID NO: 10 | Qs72S_1_opt | SEQ ID NO: 37 | UGT73-7 | — |
| KAD3640083 | SEQ ID NO: 11 | UGT72-7 | — | UGT73-8 | — |
| KAD3640083_Scopt | SEQ ID NO: 12 | UGT72-8 | — | SrUGT73E1 | SEQ ID NO: 56 |
| KAD3640084 | SEQ ID NO: 13 | UGT72-9 | — | SrUGT73E1_opt | SEQ ID NO: 57 |
| KAD3640084_Scopt | SEQ ID NO: 14 | UGT72-10 | — | XP_023746016 | SEQ ID NO: 58 |
| KAD3640181 | SEQ ID NO: 15 | XP_023875154 | SEQ ID NO: 38 | XP_023746016_Scopt | SEQ ID NO: 59 |
| KAD3640181_Scopt | SEQ ID NO: 16 | XP_023875154_Scopt | SEQ ID NO: 39 | UGT73-9 | — |
| PWA70520 | SEQ ID NO: 17 | UGT72-11 | — | UGT73-10 | — |
| PWA74166 | SEQ ID NO: 18 | XP_023876189 | SEQ ID NO: 40 | UGT73-11 | — |
| PWA79737 | SEQ ID NO: 19 | XP_023876282 | SEQ ID NO: 41 | UGT73-12 | — |
| PWA89810 | SEQ ID NO: 20 | UGT72-12 | — | XP_023764656 | SEQ ID NO: 60 |
| SrUGT71E1 | SEQ ID NO: 21 | XP_023905565 | SEQ ID NO: 42 | UGT73-14 | — |
| SrUGT71E1_opt | SEQ ID NO: 22 | XP_023905565_Scopt | SEQ ID NO: 43 | UGT73-15 | — |
| 71UGT-5 | — | XP_023914549 | SEQ ID NO: 44 | UGT73-16 | — |
| XP_021998812 | SEQ ID NO: 23 | XP_023914549_Scopt | SEQ ID NO: 45 | UGT73-17 | — |
| XP_021998812_Scopt | SEQ ID NO: 24 | XP_023923919 | SEQ ID NO: 46 | UGT73-18 | — |
| 71UGT-6 | — | UGT72-13 | — | UGT73-19 | — |
| XP_023770763 | SEQ ID NO: 25 | UGT72-14 | — | UGT73-20 | — |
| XP_023770764 | SEQ ID NO: 26 | UGT72-15 | — | | |
| XP_023770764_Scopt | SEQ ID NO: 27 | UGT72-16 | — | | |
| 71UGT-7 | — | XP_030967178 | SEQ ID NO: 47 | | |
| XP_027172778 | SEQ ID NO: 28 | XP_030967178_Scopt | SEQ ID NO: 48 | | |
| XP_028099977 | SEQ ID NO: 29 | UGT72-17 | — | | |
| XP_028101314 | SEQ ID NO: 30 | | | | |

Table 5 lists the UGTs expressed in E. coli. These were subsequently purified and tested for their ability to glucosylate Oripavine and/or Nororipavine.

### HPLC Analysis for Example 1 to 6

Stock solutions of oripavine and nororipavine were prepared in DMSO at a concentration of 10 mM. Standard solutions were prepared at concentrations of 50 μM, 100 μM, 250 μM and 500 μM from the stock solutions.

1 μL samples of standard solution or UGT test sample were injected into an Agilent 1290 Infinity I UHPLC with a binary pump (Agilent Technologies, Palo Alto, CA, USA) at a mobile phase flow rate at 600 μL/min. Separation was achieved on a Kinetex F5 column (100×2.1 mm, 1.7 μm, 100 Å, Phenomenex, Torrance, CA, USA) using 0.05% (v/v) formic acid in H₂O and 0.05% (v/v) formic acid in acetonitrile as mobile phases A and B, respectively using the time-gradient as shown in Table 6

TABLE 6

| Time (min) | % B |
|---|---|
| 0.0-3.6 | 2-30 |
| 3.6-4.1 | 30-100 |
| 4.1-5.1 | 100 |
| 5.1-5.5 | 100-2 |
| 5.5-6.5 | 2 |

The column temperature was maintained at 30° C. The liquid chromatography system was coupled to an Agilent 1290 diode array detector (Agilent Technologies, Palo Alto, CA, USA). UV-spectra were acquired at 220, 254 and 285 nm with 285 nm used for quantification of nororipavine and oripavine.

### LC/MS Analysis

Samples containing O-glycosyl Nororipavine and O-glycosyl Oripavine were injected into an Agilent 1290 Infinity II UHPLC with a binary pump coupled to an Ultivo QqQ mass spectrometer (Agilent Technologies, Palo Alto, CA, USA). Separation was achieved on a Kinetex F5 column (100×2.1 mm, 1.7 μm, 100 Å, Phenomenex, Torrance, CA, USA) using 0.1% (v/v) formic acid in H₂O and 0.1% (v/v) formic acid in acetonitrile as mobile phases A and B, respectively. The gradient was as shown in Table 7. Specific conditions for UHPLC and MS can be found in Table 8 and Table 9. O-glycosyl Nororipavine and O-glycosyl Oripavine were detected in multiple reaction monitoring (mode) specified in Table 10.

TABLE 7

| Time (min) | % B |
|---|---|
| 0 | 2 |
| 0.30 | 2 |
| 4.00 | 30 |
| 4.40 | 100 |
| 4.90 | 100 |
| 5 | 2 |
| 6 | 2 |

TABLE 8

| Parameter | Value |
|---|---|
| Injection volume | 2 μl |
| Column Temperature | 30° C. ± 4° C. |
| Injection method | Flow through needle |
| Flow | 0.4 ml/min |
| Auto sampler temperature | 4° C. ± 2° C. |
| Reconditioning wash | 2% Acetonitrile (in H₂O), 5 sec |
| Weak wash | 20% Methanol (in H₂O), 5 sec |
| Strong wash | 30% Acetonitrile, 30% Methanol, |
| | 30% 2-Propanol, 10% H₂O, 10 sec |
| Seal wash | 20% 2-Propanol (in H₂O) |

TABLE 9

| Source Parameter | Value | |
|---|---|---|
| Ion Source | Electrospray | |
| | Positive Mode (ESI+) | |
| Capillary Voltage | 3.5 | kV |
| Nozzle Voltage | 500 | V |
| Source Gas Temperature | 340° C. | |
| Source Gas Flow | 12 | L/min |
| Source Sheath Gas Temperature | 380° C. | |
| Source Sheath Gas Flow | 12 | L/min |
| Nebulizer | 30 | psi |

| | |
|---|---|
| Mode | MS/MS |

TABLE 10

| | Retention | Precursor | Product | Fragmentor | Collision |
|---|---|---|---|---|---|
| Target | time | ion | ion | voltage | energy |
| compound | (min) | (m/z) | (m/z) | (V) | (V) |
| O—glycosyl | 2 | 446 | 284 | 120 | 10 |
| nororipavine | | | 267 | 120 | 10 |
| O—glycosyl | 2.1 | 460 | 298 | 110 | 0 |
| oripavine | | | 58.1 | 110 | 5 |

### HPLC Analysis for Example 7 to 9

Stock solutions of oripavine and nororipavine were prepared in DMSO at a concentration of 10 mM. Standard solutions were prepared at concentrations of 50 μM, 100 μM, 250 μM and 500 μM from the stock solutions. Samples were injected into an Agilent 1290 Infinity I UHPLC with a binary pump (Agilent Technologies, Palo Alto, CA, USA). Separation was achieved on a Kinetex F5 column (100×2.1 mm, 1.7 μm, 100 Å, Phenomenex, Torrance, CA, USA) using 0.05% (v/v) formic acid in H₂O and 0.05% (v/v) formic acid in acetonitrile as mobile phases A and B, respectively using the time-gradient as shown in Table 11

TABLE 11

| Time (min) | % B |
|---|---|
| 0.0-3.6 | 2-30 |
| 3.6-4.1 | 30-100 |
| 4.1-5.1 | 100 |
| 5.1-5.5 | 100-2 |
| 5.5-6.5 | 2 |

The injection volume was 1 μL and the mobile phase flow rate was 600 μL/min. The column temperature was maintained at 30° C. The liquid chromatography system was coupled to an Agilent 1290 diode array detector (Agilent Technologies, Palo Alto, CA, USA). UV-spectra were acquired at 220, 254 and 285 nm with 285 nm used for quantification of nororipavine and oripavine.

In subsequent assays a modified HPLC method was utilized. Stock solutions were prepared in DMSO at a concentration of 25 mM. Standard solutions were prepared at concentrations of 50 μM, 125 μM, 250 μM and 500 μM from the stock solutions. Samples were injected into an Agilent 1290 Infinity I UHPLC with a binary pump (Agilent Technologies, Palo Alto, CA, USA). Separation was achieved on a Kinetex F5 column (100×2.1 mm, 1.7 μm, 100 Å, Phenomenex, Torrance, CA, USA) using 0.05% (v/v) formic acid in H₂O and 0.05% (v/v) formic acid in acetonitrile as mobile phases A and B, respectively using the time-gradient as shown in Table 12

TABLE 12

| Time (min) | % B |
|---|---|
| 0.0-1.0 | 2 |
| 1.0-4.8 | 2-30 |
| 4.8-5.0 | 30-100 |
| 5.0-6.0 | 100 |
| 6.0-6.2 | 100-2 |
| 6.2-6.5 | 2 |

O-glycosyl nororipavine, 0-glycosyl oripavine, nororipavine and oripavine eluted at 2.13, 2.40, 2.73 and 2.97 min, respectively.

The injection volume was 1 μL and the flow rate was 600 μL/min. The column temperature was maintained at 30° C. The liquid chromatography system was coupled to an Agilent 1290 diode array detector (Agilent Technologies, Palo Alto, CA, USA). UV-spectra were acquired at 220, 254 and 285 nm. 285 nm used for the quantification of nororipavine, oripavine and 0-glycosyl-compounds.

### LC/MS Analysis

Samples containing O-glycosyl Nororipavine and O-glycosyl Oripavine were injected into an Agilent 1290 Infinity II UHPLC with a binary pump coupled to an Ultivo QqQ mass spectrometer (Agilent Technologies, Palo Alto, CA, USA). Separation was achieved on a Kinetex F5 column (100×2.1 mm, 1.7 μm, 100 Å, Phenomenex, Torrance, CA, USA) using 0.1% (v/v) formic acid in H₂O and 0.1% (v/v) formic acid in acetonitrile as mobile phases A and B, respectively. The gradient was as shown in Table 7. Specific conditions for UHPLC and MS can be found in Table 8 and Table 9. O-glycosyl Nororipavine and O-glycosyl Oripavine were detected in multiple reaction monitoring (mode) specified in Table 10.

TABLE 13

| Time (min) | % B |
|---|---|
| 0 | 2 |
| 0.30 | 2 |
| 4.00 | 30 |
| 4.40 | 100 |
| 4.90 | 100 |
| 5 | 2 |
| 6 | 2 |

TABLE 14

| Parameter | Value |
|---|---|
| Injection volume | 2 μl |
| Column Temperature | 30° C. ± 4° C. |
| Injection method | Flow through needle |
| Flow | 0.4 ml/min |
| Auto sampler temperature | 4° C. ± 2° C. |
| Reconditioning wash | 2% Acetonitrile (in H₂O), 5 sec |
| Weak wash | 20% Methanol (in H₂O), 5 sec |
| Strong wash | 30% Acetonitrile, 30% Methanol, |
| | 30% 2-Propanol, 10% H₂O, 10 sec |
| Seal wash | 20% 2-Propanol (in H₂O) |

TABLE 15

| Source Parameter | Value | |
|---|---|---|
| Ion Source | Electrospray | |
| | Positive Mode (ESI+) | |
| Capillary Voltage | 3.5 | kV |
| Nozzle Voltage | 500 | V |
| Source Gas Temperature | 340° C. | |
| Source Gas Flow | 12 | L/min |
| Source Sheath Gas | 380° C. | |
| Temperature | | |
| Source Sheath Gas Flow | 12 | L/min |
| Nebulizer | 30 | psi |

| | |
|---|---|
| Mode | MS/MS |

TABLE 16

| | Retention | Precursor | Product | Fragmentor | Collision |
|---|---|---|---|---|---|
| Target | time | ion | ion | voltage | energy |
| compound | (min) | (m/z) | (m/z) | (V) | (V) |
| O—glycosyl | 2 | 446 | 284 | 120 | 10 |
| Nororipavine | | | 267 | 120 | 10 |
| O—glycosyl | 2.1 | 460 | 298 | 110 | 0 |
| Oripavine | | | 58.1 | 110 | 5 |

### Example 1—Expression of UGTs in E. coli, and In Vitro Tests of Glucosylation of Oripavine and Nororipavine

Preparation of Transformed E. coli Hosts

E. coli hosts expressing UGTs comprised in Table 5 was prepared by introducing the appropriate genes in the background E. coli strain using methods commonly known in the art.

### Day 1—Preparation of Overnight Cultures

96-deep-well replica plates with LB medium+Ampicillin (1 ml/well) were set up and inoculated with E. coli hosts expressing each of the UGTs. The inoculates were grown overnight at 37° C., shaking vigorously at 250 rpm.

### Day 2—Preparation of Expression Cultures

670 μL expression medium was dispensed in each well of fresh 96-deep-well plates followed by addition of 3304 overnight culture. The expression cultures were incubated in the wells overnight at 20° C. and shaking at 180-250 rpm.

### Day 3— Harvesting and Resuspension:

Cell of the expression cultures were harvested by centrifugation (max speed, 5 minutes, room temperature) and discarding the supernatant. The cells were resuspended each cell pellet in 1254 cold resuspension medium 50 mM Tris/HCl (pH7.4) containing 1 mM PMSF and EDTA-free Complete mini, protease inhibitor (Roche). Preferably, the PMSF/protease inhibitor is added to the buffer just prior to resuspension due to the limited stability of PMSF/protease inhibitor in aqueous solution. After resuspension the samples were frozen at −20° C. for later lysis and purification of UGT protein.

### Example 2—Purification of UGTs from E. coli Expression Cultures

Crude protein extracts were prepared by thawing the the frozen samples from example 1 at 30° C. for 5 minutes, followed by addition of lysozyme to lyse cells. Upon lysis of cells the lysate became viscous due to release of of genomic DNA. Samples were incubated on ice for 30 min to ensure cell lysis.

UGTs in the samples were isolated using His MultiTrap HP plates (GE Healthcare) according to instructions provided with the plates and using ice stored binding and elution buffers. Double eluates of a total volume of 250 μL were gently mixed with 250 μL pure glycerol and stored at −20° C.

### Example 3—In Vitro Test of Glucosylation of Oripavine and Nororipavine by UGTs

Glucosylation samples of a 20 μL total reaction volume for each UGT to be tested were prepared in 96-well microtiter plates as follows:

TABLE 17

| | | |
|---|---|---|
| Purified UGT enzyme of example 2 | 5 | μl |
| 25 mM Oripavine or Nororipavine | 0.4 | μl |
| 1 M Tris-HCl, 50 mM MgCl2, | 2 | μl |
| 10 mM KCl pH 7.4 | | |
| Milli-Q H2O | 11.9 | μl |
| FastAP phosphatase (1 U/μL) | 0.2 | μl |
| ThermoFisher Scientific | | |
| (catalog no. EF0651) | | |
| 50 mM UDP-Glucose | 0.5 | μl |
| Total Volume | 20 | μl |

The microtiter plate was sealed with an adhesive seal, spun shortly and the glucosylation samples were incubated overnight at 30° C. After incubation, glucosylation reaction was terminated by adding 3 volumes (60 μL) of ice-cold 100% methanol the reaction volume and mixing well, followed by centrifugation of the plate at 3000 rpm for 5 min at 4° C. and analysis by HPLC.

### Results

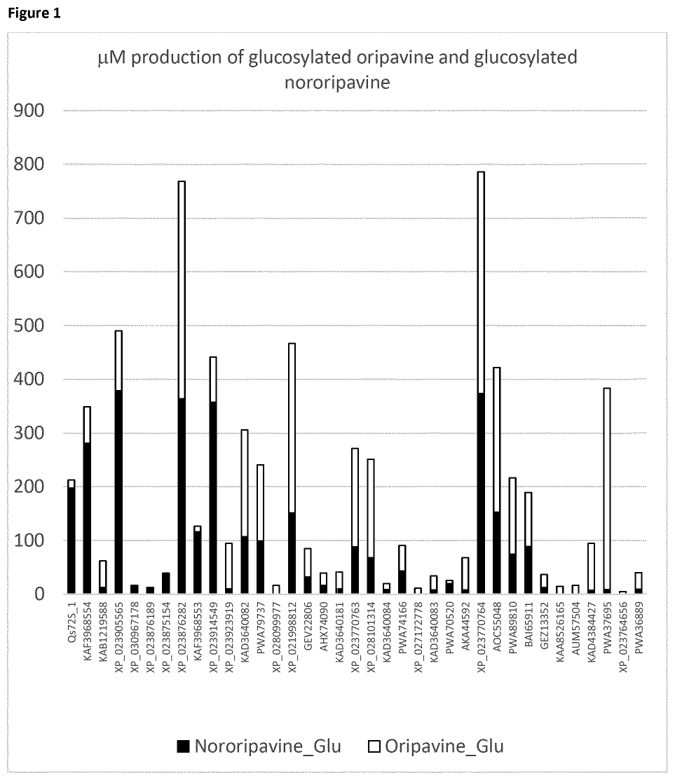

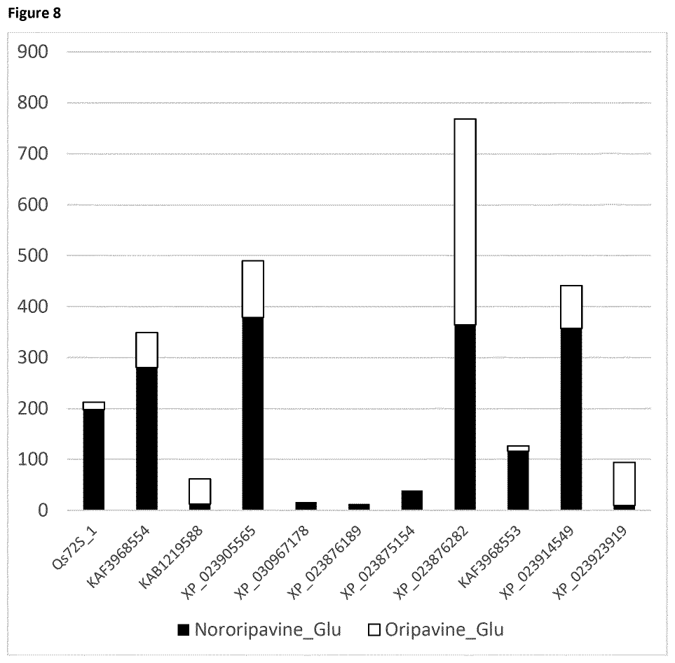

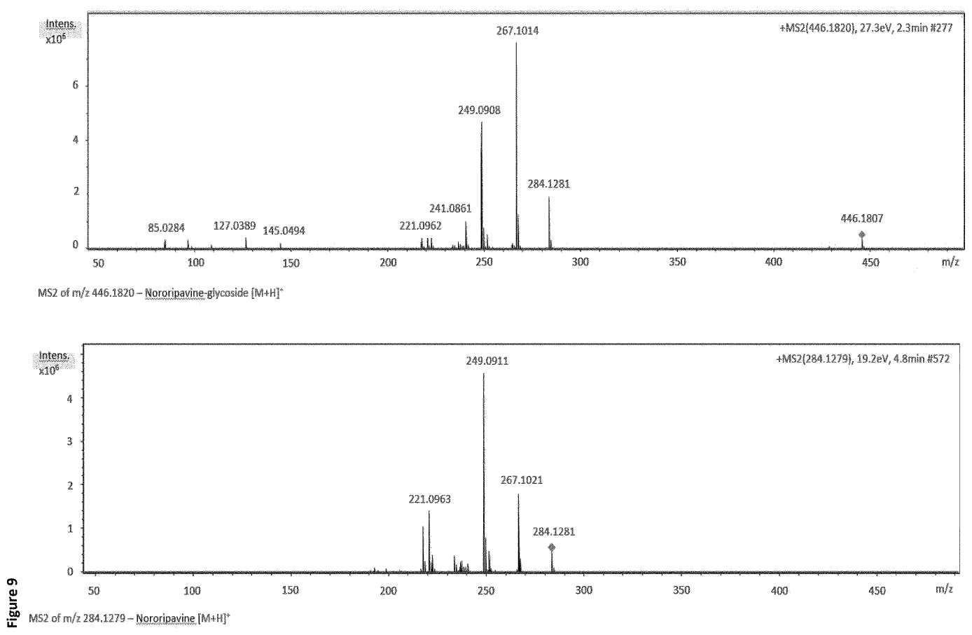

Production of glucosylated oripavine and/or nororipavine by UGTs capable of catalysing the reaction on one or both the acceptors is shown in FIG. 1 as well as for the subset of subfamily 72 UGT's in FIG. 8. It is observed that subfamily 72 UGTs Qs72S_1 (XP_023905554), KAF3968554, XP_023905565, XP_030967178, XP_023876189, XP_023875154, KAF3968553, and XP_023914549 seemed to have a particularly strong preference for Nororipavine glucosylation compared to oripavine glucosylation, especially Qs72S_1 (Quercus suber) and KAF3968553, while for subfamily 71 UGTs PWA70520 and SrUGT71E1 had preference for glucosylation of nororipavine compared to oripavine (the latter also shown by expression in yeast, see below). Subfamily 73 UGTs were generally good at both nororipavine and oripavine glucosylation, although some had a very strong preference for oripavine glucosylation, such as PWA37695.

### Example 4—Construction and Test of Yeast Strain Expressing UGTs In Vivo Glucosylating Oripavine and/or Nororipavine

A Saccharomyces cerevisiae strain, sOD504, was constructed by modifying background strain sOD157 by genomic integration using the Saccharomyces cerevisiae gene integration and expression system developed by Mikkelsen, M D et al. (2012).

The genes SEQ ID NO: 237 and SEQ ID NO: 160 were integrated into the site X-3 of the Saccharomyces cerevisiae strain using the Saccharomyces cerevisiae TDH3 and TEF2 promoters respectively to drive transcription. Selection for transformants was done using the well-known Kluyveromyces lactis LEU2 marker and growth on media lacking leucine.

Subsequently, the genes SEQ ID NO: 233 and SEQ ID NO: 160 were integrated into the site XI-2 of the Saccharomyces cerevisiae strain using the Saccharomyces cerevisiae TDH3 and TEF2 promoters respectively to drive transcription. Selection for transformants was done using the well-known Schizosaccharomyces pompe HISS marker and growth on media lacking histidine.

Finally, multiple copies of the genes SEQ ID NO: 154 and SEQ ID NO: 239 were integrated into the previous mentioned strain background by Ty integration. Method of Ty genomic integration was modified based on system developed by Maury, J et al. 2016. The SEQ ID NO: 154 and SEQ ID NO: 239 genes were expressed using the well-known Saccharomyces cerevisiae TDH3 and TEF2 promoters, respectively. Ty expression of the genes was integrated by using the Kluyveromyces lactis URA3 marker as selection marker for growth on media lacking uracil (described e.g. by Mikkelsen, M D et al. (2012).

The created sOD398 strain was then made leucine auxotrophic by replacement of the Kluyveromyces lactis LEU2 marker with the NatMX dominant marker, creating strain sOD504. sOD504 was capable of efficient demethylation of oripavine to nororipavine when oripavine was supplemented to the growth medium. This strain was used as test strain for expression of the UGT genes listed in Table 18.

Genes of UGTs from families 71, 72 and 73 were codon optimized for expression in yeast and synthesized and the UGT genes were inserted in P415-TEF vector (Mumberg D, et al. (1995)). UGT ORFs were inserted between the SpeI and XhoI sites yielding plasmids shown in the second column of Table 18.

For each plasmid transformation three individual colonies were tested. The test strains were cultivated in 96-deep-well-plate (DWP) format. Cells were grown in 0.5 ml SC-His-Leu-Ura medium at 30° C. with shaking at 280 rpm in ISF1-X Kuhner shaker for 20-24 hours and utilized as precultures for in vivo bioconversion assays. 50 μl of the overnight cell cultures were then grown in 450 μl DELFT minimal medium with 4 mM oripavine or 4 mM nororipavine and cells were grown for 72 hours with shaking at 280 rpm.

For HPLC UV analysis, 50 μl of cell cultures were transferred to a new 96-deep-well-plate containing 50 μl of MilliQ water with 0.1% of formic acid. The harvested 96 well plate was incubated at 80° C. for 10 minutes. Plate was then centrifugated for 10 minutes at 4000 rpm. The supernatants were then diluted in MilliQ water with 0.1% of formic acid to reach a final dilution of 1:40 before HPLC analysis.

TABLE 18

| # | Plasmid | UGT | subfamily | Sequence |
|---|---|---|---|---|
| E2 | pOD1921 | KAD3640082_ScOpt | 71 | SEQ ID |
| | | | | NO: 10 |
| E3 | pOD1922 | XP_023746016_ScOpt | 73 | SEQ ID |
| | | | | NO: 59 |
| E4 | pOD1923 | XP_021998812_ScOpt | 71 | SEQ ID |
| | | | | NO: 24 |
| E5 | pOD1924 | PWA36889_ScOpt | 73 | SEQ ID |
| | | | | NO: 54 |
| E6 | pOD1925 | KAD3640181_ScOpt | 71 | SEQ ID |
| | | | | NO: 16 |
| E7 | pOD1926 | KAD3640084_ScOpt | 71 | SEQ ID |
| | | | | NO: 14 |
| E8 | pOD1927 | KAD3640083_ScOpt | 71 | SEQ ID |
| | | | | NO: 12 |
| E9 | pOD1928 | XP_023770764_ScOpt | 71 | SEQ ID |
| | | | | NO: 27 |
| E10 | pOD1929 | UGT73-11 | 73 | — |
| E11 | pOD1930 | GEU38196_ScOpt | 73 | SEQ ID |
| | | | | NO: 50 |
| E12 | pOD1931 | KAD4384427_ScOpt | 73 | SEQ ID |
| | | | | NO: 52 |
| E13 | pOD1937 | KAF3968553_ScOpt | 72 | SEQ ID |
| | | | | NO: 33 |
| E14 | pOD1938 | XP_023914549_ScOpt | 72 | SEQ ID |
| | | | | NO: 45 |
| E15 | pOD1939 | KAF3968554_ScOpt | 72 | SEQ ID |
| | | | | NO: 35 |
| E16 | pOD1940 | XP_023905565_ScOpt | 72 | SEQ ID |
| | | | | NO: 43 |
| E17 | pOD1941 | XP_030967178_ScOpt | 72 | SEQ ID |
| | | | | NO: 48 |
| E18 | pOD1942 | UGT72-16 | 72 | — |
| E19 | pOD1943 | UGT72-12 | 72 | — |
| E20 | pOD1950 | XP_023875154_ScOpt | 72 | SEQ ID |
| | | | | NO: 39 |
| E21 | pOD1951 | SrUGT73E1_opt | 73 | SEQ ID |
| | | | | NO: 57 |
| E22 | pOD1952 | Qs72S1_opt | 72 | SEQ ID |
| | | | | NO: 37 |
| E23 | pOD1953 | SrUGT71E1_opt | 71 | SEQ ID |
| | | | | NO: 22 |

### Results

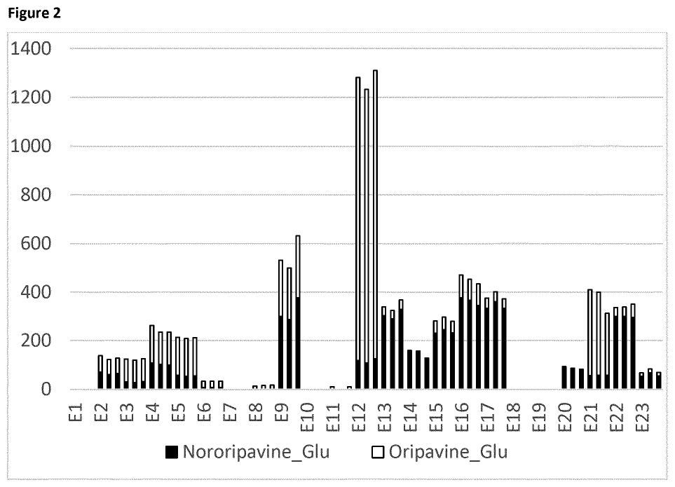

Bars in FIG. 2 show values in μM glucosylated Oripavine (Oripavine_Glu) and μM glucosylated Nororipavine (Nororipavine_Glu). UGTs from subfamilies 71, 72 and 73 were capable of glucosylating Oripavine and Nororipavine when the sOD504 strain was expressing the UGTs and oripavine was fed to the growth medium. Both subfamily 71 UGTs and subfamily 72 UGTs were shown to produce more glucosylated Nororipavine compared to glucosylated Oripavine. All subfamily 72 UGTs that were tested and shown to be active in yeast, proved to be highly specific for glucosylation of Nororipavine.

### Example 5 Construction of Yeast Strain Expressing UGT Glucosylating Nororipavine and Test of Nororipavine Glycosylation on Strain Vitality/Growth and Total Nororipavine Production

Strain sOD157 was modified into strain sOD507 by genomic integration using the Saccharomyces cerevisiae gene integration and expression system developed by Mikkelsen, M D et al. (2012).

The genes SEQ ID NO: 235 and SEQ ID NO: 160 were integrated into the site X-3 of sOD157 using the Saccharomyces cerevisiae TDH3 and TEF2 promoters respectively to drive transcription. Selection for transformants was done using the Kluyveromyces lactis LEU2 marker and growth on medium lacking leucine.

Subsequently, the genes SEQ ID NO: 255 and SEQ ID NO: 257 were integrated in multiple copies into the previous mentioned strain background by Ty integration. The method of Ty genomic integration was modified based on system developed by Maury, J et al. 2016.

The SEQ ID NO: 255 and SEQ ID NO: 257 genes were expressed using the well-known Saccharomyces cerevisiae TDH3 and TEF2 promoters respectively. Selection for Ty integration of the genes was done by using Schizosaccharomyces pompe HISS marker and growth on media lacking histidine (described e.g. by Mikkelsen, M D et al. (2012).

Finally, multiple copies of the genes SEQ ID NO: 249 and SEQ ID NO: 257 were integrated into the previous mentioned strain background by Ty integration. Method of Ty genomic integration was modified based on system developed by Maury, J et al. 2016. The SEQ ID NO: 249 and SEQ ID NO: 257 genes were expressed using the well-known Saccharomyces cerevisiae TDH3 and TEF2 promoters respectively. Ty expression of the genes was integrated by using the Kluyveromyces lactis URA3 marker as selection marker for growth on media lacking uracil (described e.g. by Mikkelsen, M D et al. (2012).

Strain sOD507 was further modified into strain sOD515 expressing the gene SEQ ID NO: 33 encoding a UGT72 shown to be capable of efficient and specific glucosylation of nororipavine. K SEQ ID NO: 33 was integrated into the site XII-2 of the Saccharomyces cerevisiae strain using the Saccharomyces cerevisiae TDH3 promoter to drive transcription. Selection for transformants was done using the well-known HygMX marker and growth on solid YPD medium containing Hygromycin.

To test if glucosylation of produced nororipavine in an oripavine-to-nororipavine converting yeast strain was beneficial for vitality/growth and total nororipavine production titer of the strain, performance of strains sOD507 and sOD515 in fed-batch fermentations supplemented with oripavine, were compared after 66 hours of fermentation.

### Fermentation Process and Fermentation Process Parameters

### Seed Train Preparation

Day 1—Preparation of pre-seeding cultures: from a frozen glycerol stock a suitable number of cells was transferred into culture tubes containing about 5 ml of seed-train medium. Culture tubes were then incubated on an orbital shaker (180 rpm) at 30° C. for c.a. 24 h in order to reach a final OD600 of about 3-4

Day 2—Preparation of seeding cultures: seeding cultures were prepared in 250 mL Erlenmeyer flasks each containing 60 mL of seed-train medium. Each flask was inoculated with a suitable amount of yeast cells which were harvested at the end of the previous propagation step. Seeding cultures were initiated with a starting OD of about 0.05 and then incubated on an orbital shaker (180 rpm) at 30° C. for c.a. 30 h in order to reach a final OD600 of about 5-6

Day 3—Inoculation of 2 liter fermentor: The batch-phase was started with a fixed working volume consisting in 500 mL of fresh broth. The fermentor was inoculated by transferring into the vessel 50 mL of the seeding culture (with an initial OD of 0,5-0,6) after removal of an equal volume of batch medium.

### Process Parameters for Batch & Fed-Batch Phases of Cultivation

The fermentation process was operated as a series of two stages carried out in the same vessel. During the first stage, that coincided with the first 8 hours of cultivation run, the yeast culture was grown batchwise in 0.5 L of batch medium: the temperature was set at 28° C. while the pH value was kept around a set point of 5.5, and automatically controlled during the cultivation by adding 12.5% ammonium hydroxide with a peristaltic pump. Fully aerobic conditions were ensured by flowing 1 vvm of air through the vessel; stirring was kept at a constant rate of 1100 rpm.

At the end of the 8 hours of batch run the second fed-batch phase was initiated by starting the glucose feed. Process parameters for the fed-batch phase were again the same used during the previous batch phase (i.e., Temperature=28° C., pH=5.5, Aeration rate=1 vvm, Stirring rate=1100 rpm). In particular, the air flow was increased stepwise in order to compensate for the increase in volume and to maintain the aeration rate value at around 1 vvm during the course of fermentation. In addition, the pH was reduced to a value of 4.5 after 64 hours from the start of the overall cultivation process. Finally, 6.0 g of Oripavine powder were fed into the cultivation vessel after approx. 66 hours of fermentation run. The addition of oripavine resulted in a pH increase, which was counterbalanced by adding phosphoric acid solution with a syringe trough an injection port.

### Feeding Strategy (Dosage, Profile, Control-Trigger)

A constant specific growth rate (μ) strategy was employed for feeding the fed-batch medium to the fermentation consisting of four consecutive exponential feeding phases where each one of them was occurring at a different specific growth rate value. The conditions for the four phases are summarized in the below Table 19:

TABLE 19

| | μᵢ | Xᵢ | Vᵢ | | Air flow |
|---|---|---|---|---|---|
| | [h⁻¹] | [g/L] | [L] | t [h] | (ccm/min] |
| Phase1 | 0.080 | 1.5 | 0.500 | (0) 8 | 620 |
| Phase2 | 0.040 | 54.32 | 0.618 | (48) 56 | 750 |
| Phase3 | 0.025 | 94.42 | 0.752 | (67) 75 | 950 |
| Phase4 | 0.012 | 162.50 | 0.947 | (90) 98 | 1300 |
| End | 0.012 | 200.61 | 1.407 | (143) 151 | 1400 |

The actual growth rate value during the fed-batch cultivation was primary controlled by the feeding rate profile of the main limiting substrate (glucose).

The actual volumetric feed rate F [L·h⁻¹, (mL·min⁻¹)] was calculated according to the following equation:

F=Fᵢ exp(μᵢt)
where
Fᵢ, initial volumetric feed rate [L·h⁻¹, (mL·min⁻¹)]
μᵢ, specific feed rate for the constant specific growth rate phase [h⁻¹]

$${F}_{i} = {μ}_{i} \frac{{X}_{i} {V}_{i}}{{Y}_{X / S} {S}_{F}}$$

with:
Xᵢ, dry microbial mass concentration in the culture vessel at the start of the phase [g·L⁻¹]
Xᵢ, volume of the culture at the start of the phase [L]
S_{F}, glucose concentration in the feed=620 g·L⁻¹
Y_{X/S}, microbial mass yield on the limiting substrate (i.e., glucose)=0.45

The transition from each one of the four phases to the next one was based on a previously optimized time profile that was able to guarantee that the system did not suffer from oxygen limitation during the course of each exponential phase.

### Results of Fed-Batch Fermentation

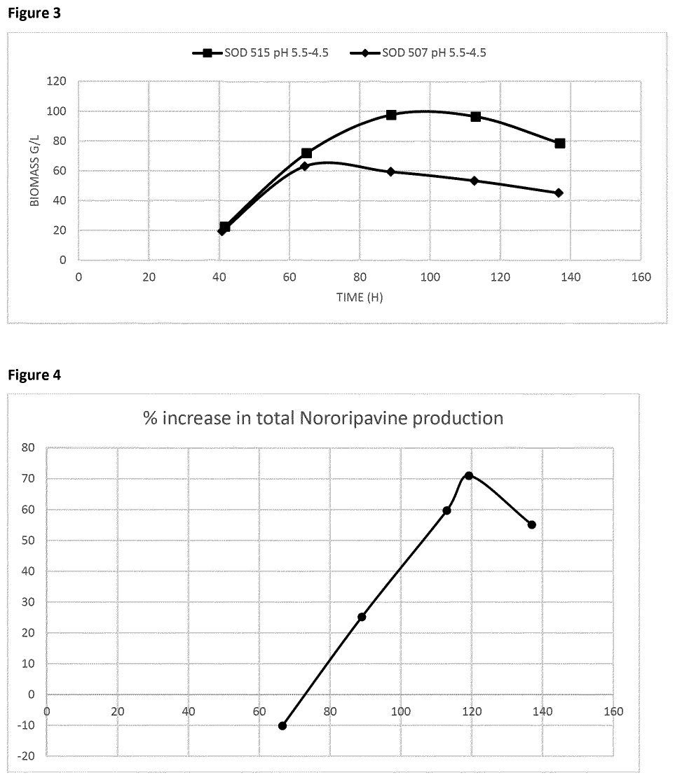

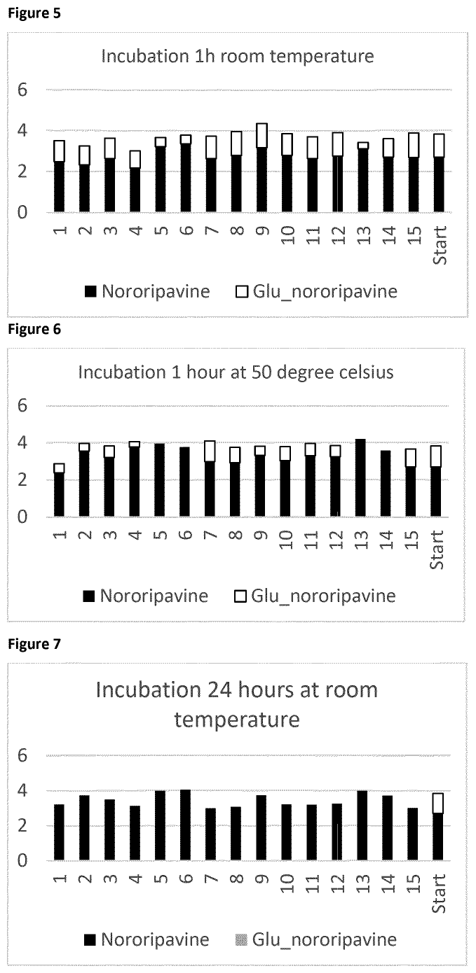

The results of the fermentations of strains sOD507 and sOD515 are shown in FIG. 3 and FIG. 4. Here it is demonstrated that once oripavine is fed to the batch after some 66 hours and the strain starts converting the oripavine to nororipavine, the expression of an UGT enzyme glucosylating the produced nororipavine dramatically increase both vitality of the strain (expressed as biomass, g/I) and production of nororipavine.

### Example 6 In Vitro Deglucosylation of Nororipavine Glucoside

Nororipavine can serve as a starting point for chemical conversion into buprenorphine and nal compounds (see eg. WO2018211331 and PCT/EP2021/050692 (unpublished), so it is desirable to provide an efficient and cost effective deglycosylation of nororipavine glucosides as part of an isolation and/or purification process. Accordingly, 15 commercially available glucosidase enzyme blends listed in Table 20 were tested for in-broth ability to deglucosylate nororipavine. 1% of enzyme blends was added to 99% broths of example 5 containing nororipavine and nororipavine glucoside and the mixtures, included a blank control (Start) were incubated at three different conditions (1 h+pH 5,5+room temperature, 1 h+pH 5,5+50° C. and 24 h+pH 5,5+room temperature). At the end of each experiment any deglucosylation activity was stopped by adding 99.9% ethanol to the broth at a ratio of 1:14.7 broth:ethanol. The broths were centrifuged at 1470 rpm for 5 min and the supernatants were diluted with 0.1% Formic acid for HPLC analysis.

TABLE 20

| Nr. | Name |
|---|---|
| 1 | Biocatalysis-Depol 40L-D040L |
| 2 | Biocatalysis-Depol 670L-D670L |
| 3 | Biocatalysis Depol 692L-D692L |
| 4 | Biocatalysis-beta-glycosidase-G016L |
| 5 | Genencor Optimate CX 15 |
| 6 | Dupont TS + E 17 |
| 7 | Novozymes-NS-11033-KTN04002 |
| 8 | Novozymes-NS-11034-KTN02243 |
| 9 | Novozymes-NS-11035-CCNF0047 |
| 10 | Novozymes-NS-11036-CNNBC028 |
| 11 | Novozymes-NS-11037-CNN02249 |
| 12 | Novozymes-NS-11038-CNNB0162 |
| 13 | Dupont TS-E 2017 |
| 14 | Novozym 188-DCN00206 |
| 15 | Viscozyme L-KTN02137 (Novozymes) |

### Results

As shown in FIG. 5, FIG. 6 and FIG. 7, several enzyme blends converted glycosylated nororipavine into nororipavine. Values shown by bars are in g/I in all three figures. The enzyme blends had a significantly increase activity at 50° C. compared to room temperature. Full deglucosylation was observed for all enzyme blends after 24 hours at room temperature. For enzyme blends 5, 6, 13 and 14 full deglucosylation was observed after only 1 hour at 50 degree Celsius.

### Example 7 Selection of UGT Having Specificity Towards Nororipavine

UDP glycosyltransferases (UGTs) are not highly homologous at the protein sequence level, although recognizable domains and binding sites are ubiquitous throughout the superfamily, and allow one to identify the proteins as UGTs (also called “signature sequences”). They are classified into families and subfamilies according to methods published by PI Mackenzie et al., 1997. Only a limited numbers of the evaluated UGTs in Table 5 displayed activity on nororipavine and these fell into the families UGT71, UGT72, and UGT73. This classification was confirmed upon blasting protein sequences against the publicly available database of Arabidopsis thaliana glycosyltransferase found at http://p450.kvl.dk/ and comparing nororipavine glycosylating UGT candidates with the Arabidopsis UGTs, and assigning the candidate into the family of the closest homolog from Arabidopsis thaliana.

The 72UGTs of Table 5 were tested in vitro to using the test setup of example 3 identify those having having specificity toward nororipavine.

### Results

The performance of the 72UGTs glucosylating oripavine and/or nororipavine is shown in FIG. 8. The 72UGTs: Qs72S_1 (XP_023905554), KAF3968554, XP_023905565, XP_030967178, XP_023876189, XP_023875154, KAF3968553, and XP_023914549 had a strong preference for nororipavine glucosylation compared to oripavine glucosylation. Qs72S_1 (Quercus suber) was particularly good. Five of these enzymes showed a particularly high specificity for nororipavine compared to oripavine: Qs72S_1, XP_030967178, XP_023876189, XP_023875154, and KAF3968553. Qs72S_1 even demonstrated both high activity and high specificity, which is particularly useful.

### Example 8 Homologies and Analysis of 72UGTs

The protein sequences for UGT72s of Table 5 where analysed and compared by full length sequence alignment.

### Results

The alignment demonstrated a significant variation in homology between the subfamily 72 UGTs. The homology as percent identity between the enzymes with glycosylation specificity towards on nororipavine were as low as 62%; although most were >74% identity to QS725_1. The enzymes with the highest activity under the conditions tested had >78% identity to QS72_1.

### Example 9 Analysis of 72UGTs Having Specificity to Nororipavine

The UGTs of FIG. 8, including the UGTs found to have the highest specificity for nororipavine, Qs725_1 (XP_023905554), KAF3968554, XP_023905565, XP_030967178, XP_023876189, XP_023875154, KAF3968553, and XP_023914549, were compared by full length alignment as shown in Table 21. The sequences of an additional 19 UGTs that were not active in this assay were also examined, and in particular 4 subfamily 72 UGT members that did not have activity for nororipavine glycosylation were compared to active UGT72 subfamily members. In the alignment the active site/binding site residues are bolded and underlined and were found to include G16, H17, 119, 1169, H172, G173, 1177, L200, 5271, H360, G362, N364, 5365, and E368, using numbering of the non-gapped QS72_1 protein sequence (SEQ ID NO: 96). Residue H17 is thought to part of the sugar acceptor binding site. Structural predictions are based on Structure 1PN3 and 1RRV crystal structures (AM Mulichak, et al, 2003 and AM Mulichak et al, 2004). It should be noted that amongst the active proteins, some conservative substitution at these sites appears to be tolerated. Whereas the H17 region is absolutely conserved, 1169 in some cases is V169 in active enzymes, and 1177 can be substituted for other aliphatic amino acids such as L, S, A and still maintain activity, but substitution with methionine was noted for several UGTs lacking activity for nororipavine as a substrate (data not shown). G173, 5271, H360, G262, N364, 5365 and E368 appear to be conserved in the proteins studied, whereas some of the proteins lacking activity in this assay have substitutions of Q, Y, K, L, or P at position 172 instead of H or M, S, Q at position 200 instead of L (data not shown).

TABLE 21

| | |
|---|---|
| Qs72_1 | ----MEQKPHIALLPSPGMGHLIPLVEFAKQFVLHH--DFHITCIIPVLGSPSKAMKAVL |
| XP_023875154 | MLSKMEQKPHIALLPSLGMGHLIPLVEFAKQFVLHH--DFHITCIIPVLGSPSKAMKAVL |
| KAF3968553 | ----MEQKPHIALLPSPGMGHLIPLVEFAKLFVLHH--DFRITCIIPVLGSPSKAMKAVL |
| XP_023914549 | ----MEQKPHIALLPSPGMGHLIPLVEFAKQFVLHH--DFRITCIVPVLGSPSKAMKAVL |
| KAF3968554 | ----MEQKPHIGILPSPGMGHLIPLVEFAKLLVHHH--DFNITCIIPVLGSPSKAMKAVL |
| XP_023905565 | ----MEQKPHIALLPSPGMGHLVPLVELAKLLLLHH--DFHITCIIPVLGSPSKAMKAVL |
| XP_030967178 | ----MEQKPHIALLPSPGMGHLIPLVELAKLLLLHH--DFHITCIIPVLGSPSKAMKAVL |
| XP_023876282 | ----MEQKPHIAIFPSPGMGHLIPHLELAKLLALHH--NFHITCIISVLGSPSRAMKEVL |
| XP_023876189 | ----MEQKPHIAILPSPGMGHLIPFVEFAKLLLLHH--DFHITCIIPTIGSPSKAMREVL |
| XP_023923919 | ----MDQKPHIALLPSPGMGHLIPLVEFAKLLLHHH--GFHITCIIPTTGSPSKAMKEVL |
| KAB1219588 | MESTHTQASHIAILPTPGMGHLIPLVEFAKRLLHHHPNSFNITFIIPTDGPPSKAQKSVL |
| | * ** * ***** * * ** ** * ** * * ** * ** |
| Qs72_1 | QALPTTIDHVFLPPVILEEEEIKGLKFEVQTILTLTRSLPPLREVLKT----TRFSAFVV |
| XP_023875154 | QALPTAIDHVFLPPVKLEEEEIKGLKFEVQTILTLTRSLPPLREVLKS----TRFSAFVV |
| KAF3968553 | QALPTTIDHVFLPPVILEEEEIKGLKFEVQTILTLTRSLPPLRDVLKS----TRFSAFVV |
| XP_023914549 | QALPTTIDHVFLPPVKLEEEEIRGLKFEVQTILTLTRSLPPLREVLKS----TRFSAFVV |
| KAF3968554 | HALPTTIDHVFLPPVILEEEEMKGPRFEVQTILTLTRSLPPLRDVLKS----TRFSAFVV |
| XP_023905565 | QALPTSIDHVFLPPVILEEEEIKGLKLEVQAMLTLTRSLPPLRDVLKS----TRFSAFVV |
| XP_030967178 | QALPTSIDNVFLPPVILEEEEIKGLKLEVQAMLTLTRSLPPLRDVLKS----TRFSAFVV |
| XP_023876282 | QALPTSIDHVFLPPV--SSEDLEGLLPEVQTILTLTRSLPPLRDVLKS----TRFAAFIV |
| XP_023876189 | QALPTSIDHVFLPPVSL--EELGGVKPGIQITLTMIRSLPPLREVLTSLVATTRLVALVV |
| XP_023923919 | QALPTSIEHVFLPPVNL--EDLKGAKPGLQIALTMTRSVPSLRDVLTSLVATTRLVALVV |
| KAB1219588 | DALPSSMNYFFLPEVNLD-DVPKGAKIESLISLTVVRSLPSLREVLKSLVESSKLVAFVV |
| | *** *** * * ** ** * ** ** * * |
| Qs72_1 | DPFGIDALDIAKELNISPYIFFPSNAFALSLIFHLPKLDETVSCEYRDLPEPLKLPGCIP |
| XP_023875154 | DPFGIDALDIAKELNISPYIFFPTNAFALSLIFHLPKLDETVSCEYRDLPEPLKLPGCIP |
| KAF3968553 | DPFGIDALDIAKELKISPYIFFPTNAFVLSLMFHLPKLDETVSCEYRDLPEPLKLPGCIP |
| XP_023914549 | DPFGIDALDIAKELNISPYIFFPTNAFALSLIFHLPKLDKTVSCEYRDLPEPLKLPGCIP |
| KAF3968554 | DPFGIDALDIAKELNISPYIFFPTNAFALSLIFHLPKLDETVSCEYRDLPEPLKLPGCIP |
| XP_023905565 | DPFGIDALDIAKELNISPYIFFPSNAFALSLVLHLPKLDETVSCEYRDLPEPIKLPGCIT |
| XP_030967178 | DPFGIDALDIAKELNISPYIFFSSNAFALSLVLHLPKLDETVPCEYRDLPEPVKLPGCIP |
| XP_023876282 | DTFGMDALDVAKEFNISPYIFFLSNAFALSLVLHLPKLDETISGEYRDQAEPLKLPGCVP |
| XP_023876189 | DLFGTDALDVAKELNVSPYIFYPTNAMVVSLVLHLPKLDETVSCEYRDLPEPVKLPGCVP |
| XP_023923919 | DPFAIDALDVAKELNVSPYIFYPANAMVLSLLLNLPKLDETVSCEYRDLPEPVKLPGCIP |
| KAB1219588 | DLFGTDAFDVARELNVSPYIFYPSTAMALSLFLYLPKLDEMVTCEYRDHPEPVKIPGCIP |
| | * * ** * * * ***** * ** ***** **** ** * *** |
| Qs72_1 | IHGRDLIEPVQDRTSELYKMFLRNAKRFRLAEGIIVNTFMELEGSAIKALLDEEAKNLPL |
| XP_023875154 | IHGRDLLEPVQDRTSELYKMFLRNAKRFRLAEGIIVNTFMELEGSAIKALLDEEAKNLPL |
| KAF3968553 | IHGRDLIEPVQDRTSELYKMFLRNVKRFRLAEGIIVNTFMELEGSAIKALLDEEAKNLPL |
| XP_023914549 | IHGRDLSEPVQDRTSELYKMFLRNAKRFRLAEGIIVNTFMELERSAIKALLDEEAKNLPL |
| KAF3968554 | IHGRDLAEPFQNRTSESYKMFLRIAKRFRLAEGIIVNTFMGLEGSAIKALLEEEAKNLPL |
| XP_023905565 | IHGRDLLEPVQDRTSELYKMFLSNTKRYRLAEGIIVNTFMELEGSAIKALLEEEAKNLPL |
| XP_030967178 | IHGRDLIEPVQDRTSELYKMFLTNAKRYRLAEGIIVNTFMELEGSAIKAMLEEEAKNLPL |
| XP_023876282 | IHGRDLAEPVQDRTSYWYKMFLRSTKRMRLAEGIMENTFMELEENAIKALLDEEAKSLPL |
| XP_023876189 | IHGRDLLDPIQDRTTELYKLFLRGAKWLRLVEGIIVNTFMELDGNVIKALEDEEAKSGTI |
| XP_023923919 | IHGRDLIDPIQDRTSEWYKLILRWAKQMRLAEGIIVNTFMELDGNAITALE-EEAKNLSL |
| KAB1219588 | IHGKDLLDPVQDRKNDAYKWLLHHAKRYRLAEGILTNSFLELEPGAIEALQAEEPGKPPV |
| | *** ** * * * ** * * ** *** * * * * * ** |
| Qs72_1 | YPIGPI-QSGSSNLQVDKSVSDCLRWLDNQPHGSVLFVCFGSGGTLSYDQTNELALGLEL |
| XP_023875154 | YPIGPI-QSGSSN-QVDKSESDCLRWLDNQPHGSVLFVCFGSGGTLSYDQTNELALGLEL |
| KAF3968553 | YPIGPI-QSGSSNLQVDKSESDCLRWLDNQPRGSVLFVCFGSGGTLSYDQTNELALGLEL |
| XP_023914549 | YPIGPI-QSGSSN-QVDKSESDCLRWLENQPHGSVLFVCFGSGGTLSYDQTNELALGLEL |
| KAF3968554 | YPVGPI-QSGSSN-QVE--ESDCLRWLGNQPHGSVLFVCFGSGGTLSYDQTNELALGLEL |
| XP_023905565 | FPVGPI-QSGSSN-QVDKSESDCLRWLDNQPHGSVLFVCFGSGGTLSYDQTNELALGLEL |
| XP_030967178 | FPVGPI-QSGSSN-QVDKLESDCLSWLDNQPHGSVLFVCFGSGGTLSYEQTNELALGLEL |
| XP_023876282 | YPIGPIIQTGSSN---QFKGSDCLRWLDSQPHGSVLFVCFGSGGTLSYEQTNELAFGLEL |
| XP_023876189 | YTIGPIIQSSSTK-HVEG--SDGLRWLDNQPRGSVLFVCFGSGGTLSYDQMKELALGLEL |
| XP_023923919 | YTVGPIIQSGSSN-QVEG--SDCLRWLNNQPSGSVLFVCFGSGGTLSYDQMNELALGLEL |
| KAB1219588 | YPVGPLVNMGSSG-KVDG--SECLKWLDEQPHGSVLFVSFGSGGTLSTNQMNELALGLEK |
| | ** * * * ** ** ****** ******** * *** *** |
| Qs72_1 | SGQKFLWVVRTPNNESADAAYLSDQILDNNPLDFLPKGFVERTEGQGLAVPSWAPQAQVL |
| XP_023875154 | SGQKFLWVVRTPNNESADAAYLSDQTLNNNLLAFLPKGFVERTEGQGLAVPSWAPQAQVL |
| KAF3968553 | SGQKFLWVVRTPDNESADAAYLSDQTLDNNPLAFLPKGFVERTEGQGLAVPSWAPQAQVL |
| XP_023914549 | SGQKFLWVVRTPNNESADAAYLSDQTLDNNPLAFLPKGFVERTEGQGLAVPSWAPQAQVL |
| KAF3968554 | SGQKFLWVVRTPNNESADAAYLSDQTLYNNPLAFLPKGFVERTEGQGLAVPSWAPQAQVL |
| XP_023905565 | SGQKFIWVVRTPNNESADATYLSDQTLGNNPLAFLPKGFVERTKGQGLVVPFWAPQAQVL |
| XP_030967178 | SGQKFIWVVRTPNNESADATYLSDQNLGDNPLAFLPKGFVERTKGQGLVVPLWAPQAQVL |
| XP_023876282 | SEQKFLWVVRTPNNESAGASYLSDQTLENNPLTSLPKGFVERTKGQGLVVPSWAPQVQVL |
| XP_023876189 | SKQKFLWVVRSPNNELAQAAYFSDQTLDNNPLSFLPVGFIERTKEQGLVVPSWAPQAQVL |
| XP_023923919 | SKQKFLWVVRSPNNGLANAAYLADQTLDNNPLAFLPKGFIERTKEQGLVVPYWAPQAQVL |
| KAB1219588 | SEQRFLWVVRTPNDHVANATYFSVQS-EKDPFDFLPKGFLERTKGRGLVLPSWAPQAQVL |
| | * * * **** * * * * * ** ** *** ** * **** *** |
| Qs72_1 | SHGSTGGFLTHCGWNSTLESIMQGIPLIAWPLYAEQKMNAPLLAEDLKVALRPKTNKSGL |
| XP_023875154 | SHGSTGGFLTHCGWNSTLESIMQGIPLIAWPLYAEQKMNAPLLAEDLKVALRPKTNKNGL |
| KAF3968553 | SHGSTGGFLTHCGWNSTLESIMQGIPLIAWPLYAEQKMNAPLLAEDLKVALRPKTNKNGL |
| XP_023914549 | SHGSTGGFLTHCGWNSILESVMQGIPLIAWPLYAEQKMNAVLLAEDLKVALRPKTNKNGL |
| KAF3968554 | SHGSTGGFLTHCGWNSSLESIMQGIPLIAWPLYAEQKMNAVLLAEDLKVALRPKTNKNGL |
| XP_023905565 | SHGSTGGFLTHCGWNSTLESIMQGIPLIVWPLFAEQKMNAALLAEDLKVALRPKTNKNGR |
| XP_030967178 | SHGSTGGFLTHCGWNSTLESIMQGIPLIVWPLFAEQKMNAALLAEDLKVALRPKTDKNGL |
| XP_023876282 | SHSSTGGFLSHCGWNSTLESVMQGIPLIAWPLFAEQRMNAVLLAEDLKVALRPKANEKGL |
| XP_023876189 | SHDSTGGFLSHCGWNSTLESIMQGIPLIAWPLFAEQRMNALLLTEDLKVALRPKANEKNL |
| XP_023923919 | SHGSTGGFLSHCGWNSTLESIMHGIPIIAWPLFAEQRMNAVLLTEDLKVALRPKANEKGL |
| KAB1219588 | SHSSTGGFLTHCGWNSILESVVNGVPLIAWPLYAEQKMNAVMLTEDIKVALRPKFNDNGL |
| | ** ****** ****** *** * * * *** *** *** * ** ******* |
| Qs72_1 | IDQEEIAKVVKGLMIGEEGKKVYNRMKDIKMAAEKALSADGSSTKALSELASQWKNHPG- |
| XP_023875154 | IDREEIAKVVKGLMIGEEGKKVYNRMKDIKMAAEKALSADGSSTKALSELASQWKNHPGF |
| KAF3968553 | IDREEIAKVVKGLMVGEEGKKVHNRMKDIKIAAEKALSADGSSTKTLSELASQWKNHPGF |
| XP_023914549 | IDREEIAKVVKGLMVGEEGKKVRNRMKDIKIAAVKALSADGSSTKALSELASQWKNHPGF |
| KAF3968554 | IDREEIAKVVKGLMDGEEGKKVRNRMKDIKIAAEKALNVDGSSTKTLSKLASKWKNHSSF |
| XP_023905565 | IDREEITKVVKGLMVGEEGKKVRNRMKDIKIAAEKALSADGSSTKALSKLATQWKNHSDF |
| XP_030967178 | IDREEIAKVVKGLMVGEEGKKVHNRMEDIKIAAEKALSADGSSTKALSKLATQWKNHPDF |
| XP_023876282 | VHRDEIAKVVKGLMVGEEGKKVHSRMKDLKDAAEKALSVDGSSTKALTDLALHWTNQARF |
| XP_023876189 | VDREEIAKVIKSLMVGEDGKKIHSRMKDLKIAAEKALSPNGPSTKALADLASHWTNQSNS |
| XP_023923919 | VDREEIAKVIKGLMVGEEGKKVHSRMKELKIAADKAVSPYGSSTKALADIASRWTNGPDF |
| KAB1219588 | AGREEIAKVAKALMEGEEGKRLRSHMKNLKDAAARVLSEDGSSTKALSELALKWKNKK-- |
| | ** ** * ** ** ** * * ** * *** * * * * |
| Qs72_1 | -- |
| XP_023875154 | -- |
| KAF3968553 | -- |
| XP_023914549 | -- |
| KAF3968554 | -- |
| XP_023905565 | -- |
| XP_030967178 | -- |
| XP_023876282 | -- |
| XP_023876189 | -- |
| XP_023923919 | RT |
| KAB1219588 | -- |

### Example 10 Effect of N-Terminal Tags on UGT Activity

A strain was constructed to test whether different N-terminal tags on a UGT had an effect on nororipavine or oripavine glucosylation efficiency or specificity. Strain sOD157, described in the Materials and Methods section, was transformed to integrate an expression cassette that expresses a CYP450 N-demethylase (SEQ ID NO: 258) from a strong constitutive promoter. Suitable promotors include for example these described in WO2021069714.

Next, this strain was transformed simultaneously with 3 different episomal CEN/ARS based yeast expression plasmids. The first plasmid was expressing the CYP450 reductase (CPR) SEQ ID NO: 260 from a strong constitutive promoter. The second plasmid was expressing a PUP (Purine Uptake Permease) from a strong constitutive promoter, suitable PUPs that work for bioconversion of oripavine to nororipavine can be found in example 12 below. The third plasmid was expressing the UGT SEQ ID NO: 97 or a modified version of these this gene with an ER anchor and a flexible linker fused in-frame to its N-terminus. Fifteen different linkers of varying length were fused to the anchor and in one case only the anchor sequence was used N-terminally to the UGT coding sequence.

Cells were grown overnight in SC-HLU and used to inoculate Delft pH 5.5 media containing 2 mM oripavine. After growth with shaking at 30 degrees Celsius for 3 days, total extracts were obtained by mixing the cultures with an equal volume of 0.2% formic acid and heating at 80 degrees Celsius for 10 minutes. After centrifugation, the supernatant was diluted in 0.1% formic acid for quantification by HPLC as described in the materials and methods.

In all 16 cases the fusion of the UGT to an ER membrane anchor lowered the activity of the expressed enzyme. Activity for glucosylation of nororipavine ranged from approximately 10-84% of the untagged UGT positive control. Surprisingly though, the N-terminal fusions to the UGT increased the specificity towards glucosylation of the nororipavine rather than the oripavine molecules. In nearly half of the experiments the specificity toward nororipavine glucosylation as compared to oripavine glucosylation was 100% within the limits of quantitation of the experiment. This was considered a great advantages as the process of N-demethylation of oripavine to nororipavine is impeded if the substrate oripavine is glucosylated and becomes unavailable for conversion to nororipavine and would create additional impurities that had to be removed in a purification process. These are factors which adversely affects the yield of the process and the cost.

Additional types of N-terminal tags were also tested. Ten different tags that impacted solubility were tested and compared to the activity with a UGT alone as described above. All 10 tagged glucosyl transferases were active. Half of those tested had higher amounts of glucosylated nororipavine than the untagged UGT alone (SEQ ID NO: 204, 208, 210, 212, 214, 216, and 218), the most active UGT fusion (SEQ ID NO: 218) had 140-150% of glucosylation activity of the untagged UGT for nororipavine and oripavine, respectively.

### Example 11 Verification of Nororipavine-Glycoside Structure by MS/MS

### A. Verification of Nororipavine-Glucoside

Extractions from strains producing glucosylated nororipavine were analysed on LC-MS-Q-TOF. Chromatography was performed on a Dionex Ultimate 3000 Quaternary Rapid Separation UHPC+ focused system (Thermo Fisher Scientific, Germering, Germany). Separation was achieved on a Kinetex 1.7 μM F5 column (100×2.1 mm, 1.7 μM, 100 Å, Phenomenex, Torrance, CA, USA), employing 0.05% (v/v) formic acid in H₂O and 0.05% formic acid in acetonitrile as mobile phases A and B, respectively. The gradient utilized was: 0-2 min 2% B; 2-13 min 2-30% B; 13-16.5 min 30-100% B; 16.5-18 min 100% B; 18-18.1 min 100-2% B; 18.1-20 min 2% B. The mobile phase flow was 300 μL/min. The column temperature was maintained at 25° C. UV-spectra were acquired at 254 and 285 nm.

The liquid chromatography system was coupled to a Compact micrOTOF-Q mass spectrometer (Bruker, Bremen, Germany) equipped with an electrospray ion source (ESI) operated in positive or negative ionisation mode. The ion spray voltage was maintained at 4500 V or 3900 V in positive or negative ionisation mode, respectively. Dry temperature was set to 200° C. and dry gas flow was set to 8 L/min. Nebulizing gas was set to 2.5 bar and collision energy to 15 eV. Nitrogen was used as a dry gas, nebulizing gas and collision gas. The m/z range was set to 50-3000. Samples were run in MS or AutoMSMS mode to acquire MS and MS/MS (MS2) spectra of all analytes present in extracts. All files were automatically calibrated by post processing to sodium formate clusters injected at the beginning of each run. The obtained mass spectra are shown in FIG. 9.

The loss from m/z 446.1807 to m/z 284.1281 is 162.0526. The calculated monoisotopic mass of a hexose-H₂O is C₆H₁₂O₆—H₂O=180.0628-18.0100=162.0528. Based on the knowledge of the expressed UGT it is known that the hexose is a glucose. The presence of fragmentation ions m/z 284.1281, 267.1014, 249.0908, and 221.0962 for parent ion m/z 446.1820 were shared with the known ions for Nororipavine as seen in the MS2 of m/z 284.1279.

In conclusion it was confirmed that the glycosylated nororipavine was in fact nororipavine with the addition of one glucose moiety.

### Structures and Masses of Nororipavine and Nororipavine-Glucoside

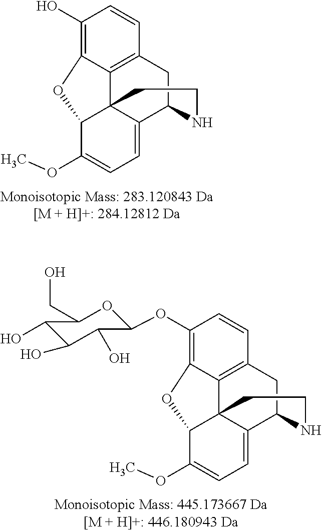 [SMILES: COC1=CC=C2[C@H]3Cc4ccc(O)c5c4[C@@]2(CCN3)[C@H]1O5 ; COC1=CC=C2[C@H]3Cc4ccc(OC5OC(CO)C(O)C(O)C5O)c5c4[C@@]2(CCN3)[C@H]1O5]

### B. Verification of the Glucosylation Position on Nororipavine.

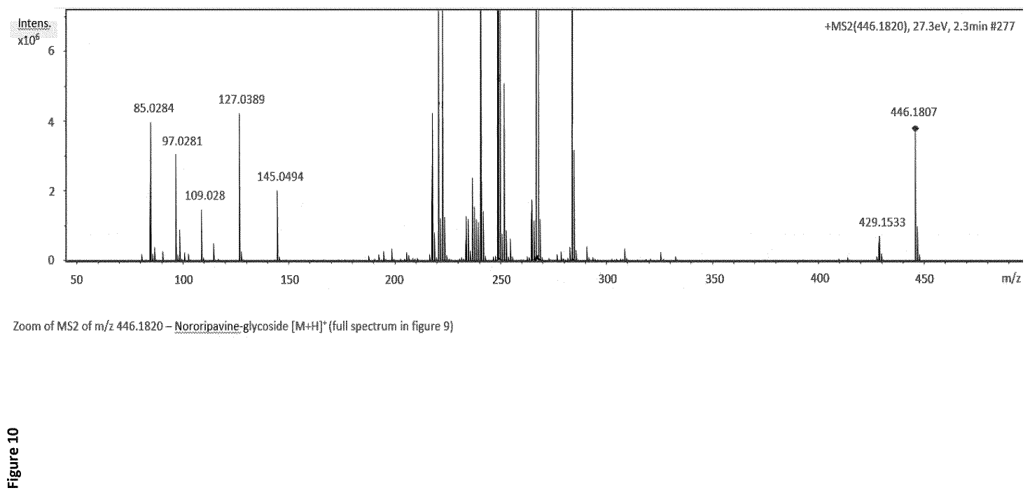

The backbone of Nororipavine has a fragmentation pattern as published for Oripavine (Zhang et al 2008/Raith et al. 2003) and it is known that the amine group of oripavine often is lost creating a fragment of m/z 267 (as seen in FIG. 10). Should the glucose unit be linked through the nitrogen it would be lost before or together with the amine group, but as can be observed in FIG. 10 a fragment of m/z 429.1533 (corresponding to 267+162), this fragment shows the presence of an ion of the Nororipavine-glycoside that has lost the amine group as shown in the formula below. Therefore the glucosylation must occur at the hydroxyl position as shown. The fragment and the calculated monoisotopic mass of m/z 429.1543 also show a high mass accuracy.

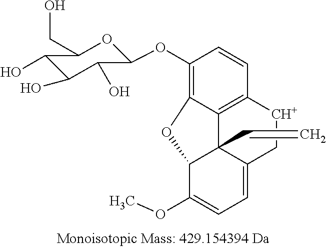

### Example 12 Demonstration of Additional Combinations of Transporters and UGTs that are Functional for Production and Glucosylation of Nororipavine

As described above in Examples 4 and 5 strains containing PUP's were used and functions well with demethylases and UGTs for production and glucosylation of nororipavine. One of skill in the art would recognize that other PUP's can also serve the same function.

A tester strain was built to demonstrate various combinations of uptake transporters and UGTs on the glucosylation of oripavine and/or nororipavine during a bioconversion reaction.

Three episomal CEN/ARS plasmids containing the cytochrome P450 (SEQ ID NO: 258), the cytochrome P450 reductase SEQ ID NO: 260 or an uptake transporter each expressed under strong constitutive promoters were simultaneously transformed into the wildtype strain sOD157 containing a UGT. For example, the UGT SEQ ID NO: 93 was integrated on the genome under a strong constitutive promoter in one experiment shown in Error! Reference source not found.

The cells were grown as in Example 4 and the samples were extracted and analysed by HPLC as described in the materials and methods. As shown in Error! Reference source not found., numerous combinations of UGTs and transporters are functional in the bioconversion reaction of oripavine to nororipavine. High levels of nororipavine glucosylation were achieved, almost full nororipavine glucosylation. Use of transporters SEQ ID NO: 246, 244, 228, 248, 250, 230, 252, and 254 all produced more than 50% glucosylation, some more than 60%, some more than 80% and some even more than 90% in cells that were fed with 0.5 mM oripavine in DELFT media pH 5.5 and grown at 30° C. with shaking at 250 rpm for 72 h.

An additional UGT (SEQ ID NO: 170) with 62% identity at the protein level to SEQ ID NO: 96 was also tested in a similar manner as above. Briefly, cells were grown overnight in SC-HLU and used to inoculate Delft pH 5.5 media containing 2 mM oripavine. After growth with shaking at 30 degrees Celsius for 3 days, total extracts were obtained by mixing the cultures with an equal volume of 0.2% formic acid and heating at 80 degrees Celsius for 10 minutes. After centrifugation, the supernatant was diluted in 0.1% formic acid for quantification by HPLC as described in the Materials and Methods. UGT SEQ ID NO: 170 was active for glucosylation on both oripavine and nororipavine however it produced 41% of the noropavine-glu relative to SEQ ID NO: 96 and 35% of oripavine-glu relative to SEQ ID NO: 96.

### REFERENCES

P. v. Hoek, E. d. Hulster, J. P. v. Dijken, J. T. Pronk. Fermentative capacity in high-cell-density fed-batch cultures of baker's yeast, Biotechnol Bioeng 68: 517-523, 2000.
Gietz RD, Schiestl RH, Mortensen U H et al. Quick and easy yeast transformation using the LiAc/SS carrier DNA/PEG method. Nat Protoc 2007; 2:35-7.
AM Mulichak, et al, “Structure of the TDP-epi-vancosaminyltransferase GtfA from the chloroeremomycin biosynthetic pathway”, Proc Natl Acad Sci USA 2003 Aug. 5; 100(16):9238-43;
AM Mulichak et al, “Crystal structure of vancosaminyltransferase GtfD from the vancomycin biosynthetic pathway: interactions with acceptor and nucleotide ligands,” Biochemistry 2004 May 11; 43(18):5170-80.
Maury J, et al. EasyCloneMulti: A Set of Vectors for Simultaneous and Multiple Genomic Integrations in Saccharomyces cerevisiae. PLoS One 11(3):e0150394, March 2 (2016).
Mikkelsen, M D et al.; Metab. Eng.; 14, Issue 2, 104-111 (2012)
Osmani et al., 2009, Phytochemistry 70: 325-347
Sonnhammer et al., Nucl. Acids Res., 26:320-322 (1998).
Sonnhammer et al., Proteins, 28:405-420 (1997).
Bateman et al., Nucl. Acids Res., 27:260-262 (1999).
Mackenzie et al, “The UDP glycosyltransferase gene superfamily: recommended nomenclature update based on evolutionary divergence,” Pharmacogenetics, 1997 August; 7(4):255-69.
Gene Reports Volume 9, December 2017, Pages 46-53: Strategies of codon optimization for high-level heterologous protein expression in microbial expression systems.
Mumberg D, et al. (1995) Gene 156(1):119-22).
Zhang, Z., Yan, B., Liu, K., Bo, T., Liao, Y., and Liu, H. (2008), Fragmentation pathways of heroin-related alkaloids revealed by ion trap and quadrupole time-of-flight tandem mass spectrometry. Rapid
Commun. Mass Spectrom., 22: 2851-2862. https://doi.org/10.1002/rcm.3686
Raith, K., Neubert, R., Poeaknapo, C., Boettcher, C., Schmidt, J. (2003), Electrospray tandem mass spectrometric investigations of morphinans. Journal of the American Society for Mass Spectrometry, 14: 1262-1269. https://doi.org/10.1016/S1044-0305(3)00539-7

1. A method for producing an oripavine glycoside and/or nororipavine glycoside comprising providing (i) a oripavine acceptor and/or nororipavine acceptor, (ii) a glycosyl donor, and (iii) a glycosyl transferase (UGT), and contacting the oripavine acceptor and/or nororipavine acceptor, the glycosyl donor, and the UGT at conditions allowing the UGT to transfer a glycosyl moiety from the glycosyl donor to the oripavine acceptor and/or nororipavine acceptor and thereby produce the oripavine glycoside and/or nororipavine glycoside.

2. The method of claim 1, wherein the glycosyl donor is a NDP-glycoside.

3. The method of claim 2, wherein the nucleoside of the nucleotide glycoside is Uridine.

4. The method of claim 3, wherein the glycosyl donor is UDP-D-glucose (UDP-Glc) or UDP-N-acetyl-D-glucosamine (UDP-GlcNAc).

5. The method of claim 1, wherein the UGT is an aglycone O-UGT.

6. The method of claim 5, wherein the UGT is an aglycone 0-glucosyltransferase.

7. The method of claim 1, wherein the UGT is derived from a plant.

8. The method of claim 7, wherein the plant is selected from the genera of Quercus, optionally Quercus suber.

9. The method of any preceding claim 1, wherein the UGT is a subfamily 71 UGT (71-UGT), a subfamily 72 UGT (72-UGT) and/or a subfamily 73 UGT (73-UGT).

10. The method of claim 9, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 170, 172, 174, 176, 178, 180, 182, 184, 186, 188, 190, 192, 194, 196, 198, 200, 202, 204, 206, 208, 210, 212, 214, 216, 218, 220, or 222.

11. The method of claim 10, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 63, 77, 81, 82, 83, 84, 86, 87, 92, 93, 94, 95, 96, 97, 98, 99, 100, 101, 102, 103, 104, 105, 107, 108, 111, 112, 115, 116, 117, 170, 172, 174, 176, 178, 180, 182, 184, 186, 188, 190, 192, 194, 196, 198, 200, 202, 204, 206, 208, 210, 212, 214, 216, 218, 220, or 222.

12. The method of claim 11, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 63, 83, 84, 86, 87, 101, 102, 103, 104, 105, 115, 170, 172, 174, 176, 178, 180, 182, 184, 186, 188, 190, 192, 194, 196, 198, 200, 202, 204, 206, 208, 210, 212, 214, 216, 218, 220, or 222.

13. The method of claim 9, wherein the UGT has a specificity towards nororipavine which at least 50% higher, such as at least 75% higher, such as at least 90% higher than the specificity towards oripavine, when performing the glycosylation in aqueous tris buffer at pH 7,4 at 30° C. and at 0.5 mM substrate level.

14. The method of claim 13, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 77, 81, 82, 92, 93, 94, 95, 96, 97, 98, 99, 100, 102, 103, 104, 105, 107, 108, 116, or 117.

15. The method of claim 14, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 102, 103, 104, or 105.

16. The method of claim 9, wherein the UGT has a specificity towards oripavine which at least 50% higher, such as at least 75% higher, such as at least 90% higher than the specificity towards nororipavine, when performing the glycosylation in aqueous tris buffer at pH 7,4 at 30° C. and at 0.5 mM substrate level.

17. The method of claim 16, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 111, 112, or 115.

18. The method of claim 17, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in SEQ ID NO: 115.

19. The method of claim 9, wherein the subfamily 71-UGT comprise an amino acid sequence which has at least 60%, such as at least 70%, such at least 75%, such as at least 80%, such as at least 90%, such as at least 95%, such as at least 99%, such as 100% identity to a 71-UGT comprised in anyone of SEQ ID NO: 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90.

20. The method of claim 19, wherein the subfamily 71-UGT comprise an amino acid sequence which has at least 60%, such as at least 70%, such at least 75%, such as at least 80%, such as at least 90%, such as at least 95%, such as at least 99%, such as 100% identity to a 71-UGT comprised in anyone of SEQ ID NO: 63, 77, 81, 82, 83, 84, 86, or 87.

21. The method of claim 9, wherein the subfamily 72-UGT comprise an amino acid sequence which has at least 60%, such as at least 70%, such at least 75%, such as at least 80%, such as at least 90%, such as at least 95%, such as at least 99%, such as 100% identity to a 72-UGT comprised in anyone of SEQ ID NO: 91, 92, 93, 94, 95, 96, 97, 98, 99, 100, 101, 102, 103, 104, 105, 106, 107, or 108.

22. The method of claim 21, wherein the subfamily 72-UGT comprise an amino acid sequence which has at least 60%, such as at least 70%, such at least 75%, such as at least 80%, such as at least 90%, such as at least 95%, such as at least 99%, such as 100% identity to a 72-UGT comprised in anyone of SEQ ID NO: 92, 93, 94, 95, 96, 97, 98, 99, 100, 101, 102, 103, 104, 105, 107, or 108.

23. The method of claim 9, wherein the subfamily 73-UGT comprise an amino acid sequence which has at least 60%, such as at least 70%, such at least 75%, such as at least 80%, such as at least 90%, such as at least 95%, such as at least 99%, such as 100% identity to a 73-UGT comprised in anyone of SEQ ID NO: 109, 110, 111, 112, 113, 114, 115, 116, 117, 118, 119, 120.

24. The method of claim 23, wherein the subfamily 73-UGT comprise an amino acid sequence which has at least 60%, such as at least 70%, such at least 75%, such as at least 80%, such as at least 90%, such as at least 95%, such as at least 99%, such as 100% identity to a 73-UGT comprised in anyone of SEQ ID NO: 111, 112, 115, 116, or 117.

25. The method of claim 9, further comprising one or more steps selected from
a) converting thebaine to oripavine;
b) converting thebaine to northebaine;
c) converting oripavine to nororipavine; and/or
d) converting northebaine to nororipavine;
by contacting the thebaine, northebaine and/or oripavine with one or more 0-demethylases and/or N-demethylases.

26. The method of claim 25, wherein the demethylase has at least 60%, such as at least 70%, such at least 75%, such as at least 80%, such as at least 90%, such as at least 95%, such as at least 99%, such as 100% identity to a to a demethylase comprised in any one of SEQ ID NO: 153 155, 157, 256, or 258.

27. The method of claim 25, further comprising the step of reducing the demethylase with a demethylase-CPR.

28. The method of claim 27, wherein the demethylase-CPR has at least 60%, such as at least 70%, such at least 75%, such as at least 80%, such as at least 90%, such as at least 95%, such as at least 99%, such as 100% identity to a to a demethylase-CPR comprised in any one of SEQ ID NO: 159, 161, or 260.

29. The method of claim 25, wherein the conversion rate (unit mass-1 time-1) of the demethylase is increased compared to the conversion rate absent any UGT's converting the oripavine and/or nororipavine and the glycosyl donor into the corresponding oripavine glycoside and/or nororipavine glycoside.

30. A method for producing an oripavine aglycone and/or nororipavine aglycone comprising providing (i) a oripavine glycoside and/or nororipavine glycoside and (ii) a glycosidase and contacting the oripavine glycoside and/or nororipavine glycoside and (ii) with the glycosidase at conditions allowing the glycosidase to catalyze separation of a glycosyl moiety from the oripavine glycoside and/or nororipavine glycoside and thereby produce the oripavine aglycone and/or nororipavine aglycone.

31. The method of claim 30, wherein the glycosidase is a β-glycosidase.

32. The method of claim 31, wherein the β-glycosidase is a β-glucosidase.

33. The method of claim 30, further comprising the steps of the method of claims 1 to 26 for providing the oripavine glycoside and/or nororipavine glycoside.

34. The method of claim 30, wherein the contacting of the oripavine acceptor and/or nororipavine acceptor, the glycosyl donor, and the UGT or the oripavine glycoside and/or nororipavine-glycoside, and the glycosidase is made in a buffered aqueous solution at a pH from 4,0 to 8,5 and at a temperature of 10 to 85° C.

35. A glycoside comprising an oripavine aglycone and/or nororipavine aglycone and a glycosyl group.

36. The glycoside of claim 35, wherein the glycosyl group is glucose.

37. The glycoside of claim 35, wherein the glycoside is an oripavine-O-glycoside or a nororipavine-O-glycoside.

38. The glycoside of claim 37, wherein the glucoside is an oripavine-O-glucoside or a nororipavine-O-glucoside.

39. A microbial host cell genetically modified to produce an oripavine glycoside and/or nororipavine glycoside in the presence of a glycosyl donor, wherein the host cell expresses one or more heterologous genes encoding one or more UGT's, which in the presence of a glycosyl donor and a oripavine acceptor and/or nororipavine acceptor, transfers a glycosyl moiety from the glycosyl donor to the oripavine acceptor and/or nororipavine acceptor and thereby produce the oripavine glycoside and/or nororipavine glycoside.

40. The host cell of claim 39, further comprising genes of a pathway producing the oripavine acceptor and/or nororipavine acceptor.

41. The host cell of claim 39, wherein the glycosyl donor is an NDP-glycoside.

42. The host cell of claim 41, wherein the nucleoside of the nucleotide glycoside is Uridine.

43. The host cell of claim 42, wherein the glycosyl donor is UDP-D-glucose (UDP-Glc) or UDP-N-acetyl-D-glucosamine (UDP-GlcNAc).

44. The host cell of claims 39 to 43, wherein the UGT is an aglycone O-UGT.

45. The host cell of claim 44, wherein the UGT is an aglycone 0-glucosyltransferase.

46. The host cell of claim 39 wherein the UGT is derived from a plant or a fungus.

47. The host cell of claim 46, wherein the plant is selected from the genera of Quercus, optionally Quercus suber.

48. The host cell of claim 39, wherein the UGT is a family 71 UGT (71-UGT), a family 72 UGT (72-UGT) and/or a family 73 UGT (73-UGT).

49. The host cell of claim 48, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 170, 172, 174, 176, 178, 180, 182, 184, 186, 188, 190, 192, 194, 196, 198, 200, 202, 204, 206, 208, 210, 212, 214, 216, 218, 220, or 222.

50. The host cell of claim 49, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 63, 77, 81, 82, 83, 84, 86, 87, 92, 93, 94, 95, 96, 97, 98, 99, 100, 101, 102, 103, 104, 105, 107, 108, 111, 112, 115, 116, 117, 170, 172, 174, 176, 178, 180, 182, 184, 186, 188, 190, 192, 194, 196, 198, 200, 202, 204, 206, 208, 210, 212, 214, 216, 218, 220, or 222.

51. The host cell of claim 50, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 63, 83, 84, 86, 87, 101, 102, 103, 104, 105, 115, 170, 172, 174, 176, 178, 180, 182, 184, 186, 188, 190, 192, 194, 196, 198, 200, 202, 204, 206, 208, 210, 212, 214, 216, 218, 220, or 222.

52. The host cell of claim 48, wherein the UGT has a specificity towards nororipavine which at least 50% higher, such as at least 75% higher, such as at least 90% higher than the specificity towards oripavine, when performing the glycosylation in aqueous tris buffer at pH 7,4 at 30° C. and at 0.5 mM substrate level.

53. The host cell of claim 52, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 77, 81, 82, 92, 93, 94, 95, 96, 97, 98, 99, 100, 102, 103, 104, 105, 107, 108, 116, or 117.

54. The host cell of claim 53, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 102, 103, 104, or 105.

55. The host cell of claim 48, wherein the UGT has a specificity towards oripavine which at least 50% higher, such as at least 75% higher, such as at least 90% higher than the specificity towards nororipavine, when performing the glycosylation in aqueous tris buffer at pH 7,4 at 30° C. and at 0.5 mM substrate level.

56. The host cell of claim 55, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in anyone of SEQ ID NO: 111, 112, or 115.

57. The host cell of claim 56, wherein the UGT comprise an amino acid sequence which has at least 60%, such as at least 70%, such at least 75%, such as at least 80%, such as at least 90%, such as at least 95%, such as at least 99%, such as 100% identity to a UGT comprised in SEQ ID NO: 115.

58. The host cell of claim 48, wherein the 71-UGT comprise an amino acid sequence which has at least 60%, such as at least 70%, such at least 75%, such as at least 80%, such as at least 90%, such as at least 95%, such as at least 99%, such as 100% identity to a 71-UGT comprised in anyone of SEQ ID NO: 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90.

59. The host cell of claim 58, wherein the 71-UGT comprise an amino acid sequence which has at least 60%, such as at least 70%, such at least 75%, such as at least 80%, such as at least 90%, such as at least 95%, such as at least 99%, such as 100% identity to a 71-UGT comprised in anyone of SEQ ID NO: 63, 77, 81, 82, 83, 84, 86, or 87.

60. The host cell of claim 48, wherein the 72-UGT comprise an amino acid sequence which has at least 60%, such as at least 70%, such at least 75%, such as at least 80%, such as at least 90%, such as at least 95%, such as at least 99%, such as 100% identity to a 72-UGT comprised in anyone of SEQ ID NO: 91, 92, 93, 94, 95, 96, 97, 98, 99, 100, 101, 102, 103, 104, 105, 106, 107, or 108.

61. The host cell of claim 60, wherein the 72-UGT comprise an amino acid sequence which has at least 60%, such as at least 70%, such at least 75%, such as at least 80%, such as at least 90%, such as at least 95%, such as at least 99%, such as 100% identity to a 72-UGT comprised in anyone of SEQ ID NO: 92, 93, 94, 95, 96, 97, 98, 99, 100, 101, 102, 103, 104, 105, 107, or 108.

62. The host cell of claim 48, wherein the 73-UGT comprise an amino acid sequence which has at least 60%, such as at least 70%, such at least 75%, such as at least 80%, such as at least 90%, such as at least 95%, such as at least 99%, such as 100% identity to a 73-UGT comprised in anyone of SEQ ID NO: 109, 110, 111, 112, 113, 114, 115, 116, 117, 118, 119, 120.

63. The host cell of claim 62, wherein the 73-UGT comprise an amino acid sequence which has at least 60%, such as at least 70%, such at least 75%, such as at least 80%, such as at least 90%, such as at least 95%, such as at least 99%, such as 100% identity to a 73-UGT comprised in anyone of SEQ ID NO: 111, 112, 115, 116, or 117.

64. The host cell of claim 39, further comprising an operative biosynthetic pathway capable of producing the oripavine acceptor and/or nororipavine acceptor, wherein the pathway comprises one or more polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a chorismate synthase converting EPSP into Chorismate;
e) a chorismate mutase converting Chorismate into prephenate;
f) a prephenate dehydrogenase (Tyr1) converting prephenate into 4-HPP;
g) an aromatic aminotransferase converting 4-HPP into L-Tyrosine;
h) a tyrosine hydroxylase (TH) converting L-tyrosine into L-dopa
i) a TH-CPR capable of reducing the TH of h);
j) a L-dopa decarboxylase (DODC) converting L-dopa into dopamine;
k) a Tyrosine decarboxylase (TYDC) converting L-dopa into dopamine;
l) a hydroxyphenylpyruvate decarboxylase (HPPDC) converting 4-HPP into 4-HPPA;
m) a monoamine oxidase converting dopamine into 3,4-DHPAA;
n) a norcoclaurine synthase (NCS) converting Dopamine and 4-HPAA into (S)-norcoclaurine;
o) a 6-O-methyltransferase (6-OMT) converting (S)-norcoclaurine into (S)-Coclaurine and/or norlaudanosoline into (S)-3′-Hydroxy-coclaurine;
p) a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
q) a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
r) a 3′-hydroxy-N-methyl-(S)-coclaurine 4′-O-methyltransferase (4′-OMT) converting (S)-3′-Hydroxy-N-Methylcoclaurine into (S)-Reticuline;
s) a 1,2-dehydroreticuline synthase-1,2-dehydroreticuline reductase (DRS-DRR) converting (S)-Reticuline into (R)-reticuline;
t) a salutaridine synthase (SAS) converting (R)-reticuline into Salutaridine;
u) a salutaridine reductase (SAR) converting Salutaridine to Salutaridinol;
v) a salutaridinol 7-O-acetyltransferase (SAT) converting Salutaridinol into 7-O-acetylsalutaridinol;
w) a thebaine synthase (THS) converting 7-O-acetylsalutaridinol or 7-O-acetylsalutaridinol acetate into thebaine;
x) a demethylase converting thebaine into oripavine, thebaine into northebaine, oripavine into nororipavine and/or northebaine into nororipavine; and/or
y) a demethylase-CPR capable of reducing the demethylase of x).

65. The host cell of claim 64, wherein the corresponding:
a) DAHP synthase has at least 70% identity to the DAHP synthase comprised in SEQ ID NO: 121
b) chorismate mutase has at least 70% identity to the chorismate synthase comprised in SEQ ID NO: 123;
c) prephenate dehydrogenase (Tyr1) has at least 70% identity to the DAHP synthase comprised in SEQ ID NO: 125
d) Tyrosine Hydroxylase (TH) has at least 70% identity to the TH comprised in SEQ ID NO: 127
e) TH-CPR has at least 70% identity to the TH-CPR comprised in SEQ ID NO: 129;
f) DODC has at least 70% identity to the DODC comprised in SEQ ID NO: 131;
g) Norcoclaurine synthase (NCS) has at least 70% identity to the NCS comprised in SEQ ID NO: 133;
h) 6-OMT has at least 70% identity to the 6-OMT comprised in SEQ ID NO: 135;
i) CNMT has at least 70% identity to the CNMT comprised in SEQ ID NO: 137;
j) NMCH has at least 70% identity to the NMCH comprised in SEQ ID NO: 139;
k) 4′-OMT has at least 70% identity to the 4′-OMT comprised in SEQ ID NO: 141;
l) DRS-DRR has at least 70% identity to the VRS_DDR comprised in SEQ ID NO:143;
m) SAS has at least 70% identity to the SAS comprised in SEQ ID NO: 145;
n) SAT has at least 70% identity to the SAR comprised in SEQ ID NO: 147;
o) SAR has at least 70% identity to the SAT comprised in SEQ ID NO: 149;
p) THS has at least 70% identity to the THS comprised in SEQ ID NO: 151;
q) Demethylase has at least 70% identity to the demethylase comprised in anyone of SEQ ID NO: 153, 155, 157, 256, or 258; and
r) Demethylase-CPR has at least 70% identity to the demethylase-CPR comprised in anyone of SEQ ID NO: 159, 161, or 260.

66. The host cell of claim 39, further comprising a demethylase converting thebaine into oripavine, thebaine into northebaine, oripavine into nororipavine and/or northebaine into nororipavine; optionally a demethylase which has at least 70% identity to a demethylase comprised in SEQ ID NO: 153, 155, 157, 256, or 258.

67. The host cell of claim 64, wherein the conversion rate (unit mass-1 time-1) of one or more pathway enzymes is increased compared to the conversion rate absent any UGT's converting the oripavine and/or nororipavine and the glycosyl donor into the corresponding oripavine glycoside and/or nororipavine glycoside.

68. The host cell of claim 39, further comprising one or more transporter proteins facilitating transport of one or more metabolites of the pathway.

69. The host cell of claim 68, wherein the transporter protein is a permease.

70. The host cell of claim 69, wherein the permease is a Purine Uptake Permease (PUP).

71. The host cell of claim 68, wherein the transporter protein has at least 70% identity to the transporter comprised in SEQ ID NO: 224, 226, 228, 230, 232, 234, 236, 238, 240, 242, 244, 246, 248, 250, 252, or 254.

72. The host cell of claim 39 expressing one or more genes selected from the group of:
a) one or more polynucleotides which is at least 70% identical to the DAHP synthase encoding polynucleotide comprised in SEQ ID NO: 122 or genomic DNA thereof;
b) one or more polynucleotides which is at least 70% identical to the chorismate mutase encoding polynucleotide comprised in SEQ ID NO: 124 or genomic DNA thereof;
c) one or more polynucleotides which is at least 70% identical to the prephenate dehydrogenase encoding polynucleotide comprised in SEQ ID NO: 126 or genomic DNA thereof;
d) one or more polynucleotides which is at least 70% identical to the TH encoding polynucleotide comprised in SEQ ID NO: 128 or genomic DNA thereof;
e) one or more polynucleotides which is at least 70% identical to the TH-CPR encoding polynucleotide comprised in SEQ ID NO: 130 or genomic DNA thereof;
f) one or more polynucleotides which is at least 70% identical to the DODC encoding polynucleotide comprised in SEQ ID NO: 132 genomic DNA thereof;
g) one or more polynucleotides which is at least 70% identical to the NCS encoding polynucleotide comprised in SEQ ID NO: 134 or genomic DNA thereof;
h) one or more polynucleotides which is at least 70% identical to the 6-OMT encoding polynucleotide comprised in SEQ ID NO: 136 or genomic DNA thereof;
i) one or more polynucleotides which is at least 70% identical to the CNMT encoding polynucleotide comprised in SEQ ID NO: 138 or genomic DNA thereof;
j) one or more polynucleotides which is at least 70% identical to the NMCH encoding polynucleotide comprised in SEQ ID NO: 140 or genomic DNA thereof;
k) one or more polynucleotides which is at least 70% identical to the 4′-OMT encoding polynucleotide comprised in SEQ ID NO: 142 or genomic DNA thereof;
l) one or more polynucleotides which is at least 70% identical to the DRS-DRR encoding polynucleotide comprised in SEQ ID NO: 144 or genomic DNA thereof;
m) one or more polynucleotides which is at least 70% identical to the SAS encoding polynucleotide comprised in SEQ ID NO: 146 or genomic DNA thereof;
n) one or more polynucleotides which is at least 70% identical to the SAT encoding polynucleotide comprised in SEQ ID NO: 148 or genomic DNA thereof;
o) one or more polynucleotides which is at least 70% identical to the SAR encoding polynucleotide comprised in SEQ ID NO: 150 or genomic DNA thereof;
p) one or more polynucleotides which is at least 70% identical to the THS encoding polynucleotide comprised in SEQ ID NO: 152 or genomic DNA thereof;
q) one or more polynucleotides which is at least 70% identical to the demethylase encoding polynucleotide comprised in anyone of SEQ ID NO: 154, 156, 158, 255, or 257 or genomic DNA thereof;
r) one or more polynucleotides which is at least 70% identical to the demethylase-CPR encoding polynucleotide comprised in any one of SEQ ID NO: 160, 162, or 259 or genomic DNA thereof; and
s) one or more polynucleotides which is at least 70% identical to the transporter encoding polynucleotide comprised in SEQ ID NO: 223, 225, 227, 229, 231, 233, 235, 237, 239, 241, 243, 245, 247, 249, 251, or 253 or genomic DNA thereof.

73. The host cell of claims 39 to 72, wherein the cell is eukaryote and selected from the group consisting of mammalian, insect, plant, or fungal cells.

74. The host cell of claim 73, wherein the cell is a plant cell of the genus Physcomitrella or Papaver or Nicotiana.

75. The host cell of claim 74, wherein the cell is a plant cell of the species Papaver soniferum or Nicotiana benthamiana.

76. The host cell of claim 73, wherein the cell is a fungal cell selected from the phylas consisting of Ascomycota, Basidiomycota, Neocallimastigomycota, Glomeromycota, Blastocladiomycota, Chytridiomycota, Zygomycota, Oomycota and Microsporidia.

77. The host cell of claim 76, wherein the fungal cell is a yeast selected from the group consisting of ascosporogenous yeast (Endomycetales), basidiosporogenous yeast, and Fungi Imperfecti yeast (Blastomycetes).

78. The host cell of claim 77, wherein the yeast cell is selected from the genera consisting of Saccharomyces, Kluveromyces, Candida, Pichia, Debaromyces, Hansenula, Yarrowia, Zygosaccharomyces, and Schizosaccharomyces.

79. The host cell of claim 78, wherein the yeast cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.

80. The host cell of claim 77, wherein the fungal cell is a filamentous fungus.

81. The host cell of claim 80, wherein the filamentous fungal cell is selected from the phylas consisting of Ascomycota, Eumycota and Oomycota.

82. The host cell of claim 81, wherein the filamentous fungal cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma

83. The host cell of claim 82, wherein the filamentous fungal cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

84. The host cell of claim 39, wherein one or more further native or endogenous genes of the cell is attenuated, disrupted and/or deleted.

85. The host cell of claim 39, wherein one or more genes of the oripavine acceptor and/or nororipavine acceptor pathway are overexpressed.

86. The cell of claim 39 further genetically modified to provide an increased amount of a substrate for at least one polypeptide of the oripavine acceptor and/or nororipavine acceptor pathway.

87. The host cell of claim 39, further genetically modified to exhibit increased tolerance towards one or more substrates, intermediates, or product molecules from the oripavine acceptor and/or nororipavine acceptor pathway.

88. The host cell of claim 39, wherein the expressed UGT is absent a signal peptide targeting the UGT for secretion.

89. The host cell of claim 39, comprising at least two copies of the genes encoding the UGT and/or any pathway enzymes.

90. The host cell of claim 39, wherein one or more native genes are attenuated, disrupted and/or deleted.

91. The host cell of claim 39, wherein host cell is a yeast strain modified by attenuating, disrupting and/or deleting one or more dehydrogenases or reductases native to the host cell comprised in anyone of SEQ ID NO: 165 or 167 or any of its paralogs or orthologs having at least 70% identity to anyone of SEQ ID NO: 165 or 167.

92. A cell culture, comprising host cell of claim 39 and a growth medium.

93. The method of claim 1 to further comprising:
a) culturing the cell culture of claim 92 at conditions allowing the host cell to produce the oripavine glycoside and/or nororipavine glycoside;
b) optionally deglycosylating the oripavine glycoside and/or nororipavine glycoside into an oripavine aglycone and/or nororipavine aglycone; and
c) optionally recovering and/or isolating the oripavine glycoside and/or nororipavine glycoside and/or the oripavine aglycone and/or nororipavine aglycone.

94. The method of claim 93, further comprising one or more elements selected from:
a) culturing the cell culture in a nutrient growth medium;
b) culturing the cell culture under aerobic or anaerobic conditions c) culturing the cell culture under agitation;
d) culturing the cell culture at a temperature of between 25 to 50° C.;
e) culturing the cell culture at a pH between 3-9;
f) culturing the cell culture for between 10 hours to 30 days;
g) culturing the cell culture under fed-batch, repeated fed-batch, continuous, or semi-continuous conditions; and
h) culturing the cell culture in the presence of an organic solvent to improve the solubility of the BIA aglycone.

95. The method of claim 93, further comprising feeding one or more exogenous oripavine acceptor and/or nororipavine acceptor or precursors thereof and/or glycoside donors to the cell culture.

96. The method of claim 93, wherein the recovering and/or isolation step comprises separating a liquid phase of host cell or cell culture from a solid phase of host cell or cell culture to obtain a supernatant comprising the oripavine glycoside and/or nororipavine glycoside by one or more steps selected from:
a) disrupting the host cell to release intracellular oripavine and/or nororipavine and/or oripavine glycoside and/or nororipavine glycoside into the supernatant;
b) separating the supernatant form the solid phase of the host cell, such as by filtration or gravity separation;
c) contacting the supernatant with one or more adsorbent resins to obtain at least a portion of the produced oripavine glycoside and/or nororipavine glycoside;
d) contacting the supernatant with one or more ion exchange or reversed-phase chromatography columns in order to obtain at least a portion of the oripavine glycoside and/or nororipavine glycoside;
e) extracting the oripavine, nororipavine, oripavine glycoside and/or nororipavine glycoside; and
f) precipitating the oripavine glycoside and/or nororipavine glycoside by crystallization or evaporating the solvent of the liquid phase; and optionally isolating the oripavine glycoside and/or nororipavine glycoside by filtration or gravity separation;
thereby recovering and/or isolating the oripavine glycoside and/or nororipavine glycoside.

97. A fermentation liquid comprising the oripavine glycosides and/or nororipavine glycosides comprised in the cell culture of claim 92.

98. The fermentation liquid of claim 97, wherein at least 50%, such as at least 75%, such as at least 95%, such as at least 99% of the host cells are disrupted.

99. The fermentation liquid of claims 97 to 98, wherein at least 50%, such as at least 75%, such as at least 95%, such as at least 99% of solid cellular material has separated from the liquid.

100. The fermentation liquid of claims 98 to 99, further comprising one or more compounds selected from:
a) precursors or products of the operative biosynthetic pathway producing the oripavine glycoside and/or nororipavine glycoside;
b) supplemental nutrients comprising trace metals, vitamins, salts, yeast nitrogen base, YNB, and/or amino acids; and
wherein the concentration of the oripavine glycoside and/or nororipavine glycoside is at least 1 mg/I liquid.

101. A composition comprising the fermentation liquid of claim 97 and/or the oripavine glycoside and/or nororipavine glycoside of claim 35 and one or more agents, additives and/or excipients.

102. The composition of claim 101, wherein the fermentation liquid and/or the oripavine glycoside and/or nororipavine glycoside have been processed into in a dry solid form.

103. The composition of claim 101, wherein the composition is in a liquid stabilized form.
